(19) United States
(12) Patent Application Publication
(10) Pub. No.: US 2010/0331571 A1
(43) Pub. Date: Dec. 30, 2010

(54) MOLDING AND METHOD FOR PRODUCING THE SAME, AND CATALYST AND METHOD FOR PRODUCING THE SAME

(75) Inventors: Hirofumi Saito, Niihama-shi (JP); Yuya Takahashi, Saijo-shi (JP); Toyohisa Hoshikawa, Niihama-shi (JP); Kazuya Tsuchimoto, Niihama-shi (JP); Osamu Yamanishi, Niihama-shi (JP)

Correspondence Address: SUGHRUE MION, PLLC, 2100 PENNSYLVANIA AVENUE, N.W., SUITE 800, WASHINGTON, DC, 20037, US

(73) Assignee: SUMITOMO CHEMICAL COMPANY, LIMITED, TOKYO (JP)

(21) Appl. No.: 12/822,894

(22) Filed: Jun. 24, 2010

(30) Foreign Application Priority Data
Jun. 24, 2009 (JP) 2009-149705
Dec. 7, 2009 (JP) 2009-277972

(51) Int. Cl. C07C 51/21; B29C 47/00; B32B 7/00; B01J 21/02; C07C 45/28; B01J 23/10; B01J 27/19; B01J 21/06; B01J 23/652; C07C 41/09; C01B 3/38; B01J 23/42; B01J 23/755; C07C 51/235; B01J 23/58

(52) U.S. Cl. 562/532; 252/373; 264/177.16; 428/120; 428/409; 502/205; 502/211; 502/249; 502/251; 502/304; 502/306; 502/307; 502/311; 502/327; 502/328; 502/335; 562/545; 568/449; 568/479; 568/698

(57) ABSTRACT

An object of the present invention is to provide a molding and a method for producing the same; a catalyst for the production of an unsaturated aldehyde and an unsaturated carboxylic acid, and a method for producing the same; and a catalyst for the production of methacrylic acid, and a method for producing the same.

The molding of the present invention shows a shape including a plurality of columnar portions disposed with a predetermined gap; and bridge portions which are provided at both ends in longitudinal directions of two adjacent columnar portions and join adjacent columnar portions each other; and including through holes surrounded by a plurality of columnar portions in the longitudinal directions of the columnar portions, and openings formed on a peripheral surface by a gap between the plurality of adjacent columnar portions. This molding can be formed by using an extrusion molding machine including a first die which has a plurality of grooves on an outer peripheral surface, and a ring-shaped or cylindrical second die fitted in the first die which has a plurality of grooves on a peripheral surface, and repeatedly rotating and stopping at least one of the first and second dies.

### BACKGROUND OF THE INVENTION

1. Field of the Invention

The present invention relates to a molding (or a molded article) useful, for example, as catalysts, catalyst carriers, adsorbents, desiccants, moisture absorbents and the like, and a method for producing the same; a catalyst for the production of an unsaturated aldehyde and an unsaturated carboxylic acid; a method for producing an unsaturated aldehyde and an unsaturated carboxylic acid using this catalyst; a catalyst for the production of methacrylic acid, and a method for producing methacrylic acid using this catalyst.

2. Description of the Related Art

It has hitherto been known that it is effective to use, as a catalyst, a complex oxide containing molybdenum, bismuth, iron, nickel and cobalt when an unsaturated aldehyde and an unsaturated carboxylic acid are produced by vapor-phase catalytic oxidation of propylene, isobutylene, tertiary butyl alcohol or the like with molecular oxygen (see Patent Reference 1 which is mentioned below).

Moldings having a columnar or cylindrical shape have been used as the catalyst as described above or catalyst carrier. These moldings have commonly been used for the catalyst reaction in a fixed bed reactor, and generally used in the method in which a reaction tube is packed with moldings as the catalysts or catalyst carriers and a gas is passed through the reaction tube.

However, when a reaction tube is packed with columnar or cylindrical moldings and a gas is passed through the reaction tube, a pressure difference between an inlet port and an outlet port of the reaction tube, i.e. a pressure loss arises. An increase in the pressure difference may cause a problem such as deterioration of selectivity of the objective product.

Therefore, the present inventors previously developed a molding having a shape in which columnar portions are joined to a spirally wound coil-shaped cylindrical material at predetermined intervals along an axial direction of the coil-shaped cylindrical material so as to solve the problem such as a pressure loss by contriving the shape of a catalyst molding (see Patent Reference 1 which is mentioned below).

This molding has an advantage that the pressure drop can be minimized even if such moldings are packed into an apparatus such as a fixed bed reactor or any other type of vessel in any direction at random. However, the molding had a problem that it is likely to be collapsed during extrusion molding, particularly cutting immediately after being molded owing to its structure.

There was also a fear of the breakage of the molding, when an apparatus such as a fixed bed reactor or any other type of vessel such as a reaction tube is packed with the moldings, which leads to the pressure loss.

Patent Reference 1: Japanese Unexamined Patent Publication (Kokai) No. 2007-117866

Patent Reference 2: Japanese Unexamined Patent Publication (Kokai) No. 2008-201130

### SUMMARY OF THE INVENTION

Therefore, a main object of the present invention is to provide a molding having a high strength as well as a method for producing the same, which molding causes a smaller pressure loss when such moldings are packed into an apparatus such as a fixed bed reactor or any other type of vessel, and also which molding is less likely to collapse or break even during cutting step in the production process of the moldings and packing the moldings into various containers.

Another object of the present invention is to provide a catalyst, made of the above mentioned molding, for the production of an unsaturated aldehyde and an unsaturated carboxylic acid; and also to provide a method capable of producing an unsaturated aldehyde and an unsaturated carboxylic acid in a satisfactory yield.

A further object of the present invention is to provide a catalyst, made of the above mentioned molding, for the production of methacrylic acid; and also to provide a method capable of methacrylic acid in a satisfactory yield.

The present inventors have intensively studied so as to achieve the above objects, thus leading to new findings that, in the case wherein moldings are used each of which molding includes a plurality of columnar portions with adjacent two columnar portions being disposed at a predetermined interval and a bridge portion which joins the adjacent columnar portions to each other, since through holes and openings are formed over the entire surface of the molding, a pressure loss leads to being minimized; and also since the molding has a sufficient strength owing to its structure, the molding is less likely to be collapsed even if a just molded article is cut into the molding immediately after a molding step, which enables the industrial production of the moldings; and furthermore, since the breakage is minimized even if an apparatus such as a fixed bed reactor or any other type of vessel is packed with the moldings, there would not lead to a risk in increasing of the pressure loss.

Also, the present inventors have found that an unsaturated aldehyde and an unsaturated carboxylic acid are produced in a satisfactory yield, based on the above findings, by using a catalyst for the production of the unsaturated aldehyde and the unsaturated carboxylic acid, the catalyst being made of the above mentioned molding and containing a specific complex oxide (or compound oxide) as a catalyst component, and also that methacrylic acid is produced in a satisfactory yield in the same manner as described above by using a catalyst for the production of methacrylic acid, the catalyst being made of the above mentioned molding and containing a heteropoly acid compound comprising at least phosphorus and molybdenum as a catalyst component, and thus the present invention has been completed.

That is, the molding according to the present invention is characterized in that it includes a plurality of (at least two, for example two, three, four, five or six) columnar portions disposed with at least one gap; and bridge portions each of which is disposed at least each ends in longitudinal directions of adjacent two columnar portions of said plurality of columnar portions respectively, and each of which joins the adjacent columnar portions to each other at their ends; and also includes through holes surrounded by the plurality of columnar portions and openings formed on a peripheral surface of the molding by gaps between the adjacent columnar portions.

In other words, the above mentioned molding according to the present invention is characterized in that it includes:

at least two circular portions each of which defines each of the above mentioned through holes and adjacent two of which are separated from each other by a predescribed distance in a molding direction of the molding, and

at least two columnar portions each of which is located between thus separated circular portions and each of which is connected, at its both ends, to the separated circular portions so that each of the circular portions are divided into the above mentioned bridge portions and the above mentioned gap is formed by the two adjacent columnar portions and the bridge portions to which the adjacent columnar portions are connected. It is noted that the plurality of columnar portions are preferably formed equianglarly around the peripheral surface of the molding.

The method for producing a molding according to the present invention includes:

using an extrusion molding machine including a first die which has a plurality of grooves on (at least two, for example two, three, four, five or six) its outer peripheral surface and a ring-shaped or cylindrical second die in which the first die is fitted and which has a plurality of grooves (at least two, for example two, three, four, five or six) on its inner peripheral surface, and

forming the molding by repeating:

(i) rotating at least one of the first and second dies from a position where the grooves of the first and the grooves of the second dies are laid one upon another to a next position where the grooves of the first and the grooves of the second dies are laid one upon another position so as to form the bridge portion;

(ii) then, stopping the rotation of one of the first and second dies and forming the columnar portions; and

(iii) rotating at least one of the first and second dies again to a position where the grooves of the first and the grooves of the second dies are laid one upon another to form the further bridge portions.

The columnar portions which have been extruded through the extrusion molding machine are cut into a predetermined length including the bridge portions.

The grooves of each of the dies are preferably formed equianglarly around the periphery surface, the number of the grooves of the first die may be the same as or different from that of the second die.

The catalyst for the production of an unsaturated aldehyde and an unsaturated carboxylic acid of the present invention includes the following embodiments:

(1) A catalyst for the production of the unsaturated aldehyde and the unsaturated carboxylic acid characterized in that:

the catalyst is made of a molding which includes a plurality of columnar portions disposed with at least one gap; and bridge portions each of which is disposed at least each ends in longitudinal directions of adjacent two columnar portions of said plurality of columnar portions respectively, and each of which joins the adjacent columnar portions to each other at their ends; and also includes through holes surrounded by the plurality of columnar portions and openings formed on a peripheral surface of the molding by gaps between the adjacent columnar portions, and

a catalyst component of the catalyst is a complex oxide which contains, in addition to at least molybdenum, bismuth and iron, nickel and/or cobalt;

(2) The catalyst for the production of an unsaturated aldehyde and an unsaturated carboxylic acid according to embodiment (1), wherein the complex oxide is represented by the following general formula (I):

MoₐBi_{b}Fe_{c}A_{d}BₑC_{f}D_{g}Oₓ (I)

wherein Mo, Bi and Fe represents molybdenum, bismuth and iron, respectively, A represents nickel and/or cobalt, B represents an element selected from manganese, zinc, calcium, magnesium, tin and lead, C represents an element selected from phosphorus, boron, arsenic, tellurium, tungsten, antimony, silicon, aluminum, titanium, zirconium and cerium, D represents an element selected from potassium, rubidium, cesium and thallium, 0<b≦10, 0<c≦10, 1≦d≦10, 0≦e≦10, 0≦f≦10 and 0<g≦2 when a=12, and X is a value determined by the oxidation state of each element;

(3) The catalyst for the production of an unsaturated aldehyde and an unsaturated carboxylic acid according to embodiment (1) or (2), wherein the complex oxide is obtained by firing (or calcining) a precursor thereof under an atmosphere of molecular oxygen-containing gas and then subjecting to a heat treatment in the presence of a reducing substance;

(4) The catalyst for the production of an unsaturated aldehyde and an unsaturated carboxylic acid according to embodiments (3), wherein the firing operation is carried out at 300° C. to 600° C.;

(5) The catalyst for the production of an unsaturated aldehyde and an unsaturated carboxylic acid according to embodiment (3) or (4), wherein the heat treatment is carried out at 200° C. to 600° C.; and

(6) The catalyst for the production of an unsaturated aldehyde and an unsaturated carboxylic acid according to any one embodiments of (3) to (5), wherein the reducing substance is a compound selected from hydrogen, ammonia, carbon monoxide, a hydrocarbon having 1 to 6 carbon atoms, an alcohol having 1 to 6 carbon atoms, an aldehyde having 1 to 6 carbon atoms and an amine having 1 to 6 carbon atoms.

In the method for producing an unsaturated aldehyde and an unsaturated carboxylic acid according to the present invention, a compound selected from propylene, isobutylene and tertiary butyl alcohol and molecular oxygen are subjected to vapor-phase catalytic oxidation in the presence of the catalyst according to any one of embodiments (1) to (6).

The catalyst for the production of methacrylic acid of the present invention includes the following embodiments:

(I) A catalyst for the production of methacrylic acid, characterized in that

the catalyst is made of a molding which includes a plurality of columnar portions disposed with at least one gap; and bridge portions each of which is disposed at least each ends in longitudinal directions of adjacent two columnar portions of said plurality of columnar portions respectively, and each of which joins the adjacent columnar portions to each other at their ends; and also includes through holes surrounded by the plurality of columnar portions and openings formed on a peripheral surface of the molding by gaps between the adjacent columnar portions, and

a catalyst component comprises a heteropoly acid compound containing at least phosphorus and molybdenum;

(II) The catalyst for the production of methacrylic acid according to embodiment (I), wherein the heteropoly acid compound further contains vanadium, at least one kind of an element selected from potassium, rubidium, cesium and thallium, and at least one kind of an element selected from copper, arsenic, antimony, boron, silver, bismuth, iron, cobalt, zinc, lanthanum and cerium;

(III) The catalyst for the production of methacrylic acid according to embodiment (I) or (II), wherein the heteropoly acid compound is obtainable by first firing of a precursor thereof under an atmosphere of non-oxidizing gas at 400° C. to 500° C. and second firing under an atmosphere of an oxidizing gas at 300° C. to 400° C.; and

(IV) The catalyst for the production of methacrylic acid according to (I) or (II), wherein the heteropoly acid compound is obtainable by first firing of a precursor thereof under an atmosphere of an oxidizing gas at 300° C. to 400° C. and second firing under an atmosphere of a non-oxidizing gas at 400° C. to 500° C.

In the method for producing methacrylic acid of the present invention, at least one kind of a compound selected from methacrolein, isobutylaldehyde, isobutane and isobutyric acid, and molecular oxygen are subjected to vapor-phase catalytic oxidation in the presence of the catalyst according to any one of embodiments (I) to (IV).

According to the molding of the present invention, since the through holes and openings are formed over the entire molding, it is possible to exert the effect of minimizing the pressure drop even if such moldings are packed into an apparatus such as a fixed bed reactor or any other kind of vessel. Also, such moldings are easily provided by an extrusion molding method.

Furthermore, according to the molding of the present invention, since the adjacent columnar portions are joined to each other by the bridge portion, the strength of the molding is improved. Therefore, it is possible to exert the effect that the moldings are less likely to be collapsed when the moldings are produced by cutting its precursor immediately after extrusion of such precursor, and are also less likely to be broken when they are packed into an apparatus such as a fixed bed reactor or any other kind of vessel such as a reaction tube.

Therefore, the moldings of the present invention are useful as catalysts, catalyst carriers, adsorbents, desiccants, moisture absorbents and the like. Particularly, the moldings can efficiently show high catalytic performances when they are used as the catalysts or the catalyst carriers.

In addition, the catalyst for the production of an unsaturated aldehyde and an unsaturated carboxylic acid, which is made of the molding as mentioned above and contains the complex oxide containing at least molybdenum, bismuth, iron, nickel and cobalt as a catalyst component, has the effect capable of producing an unsaturated aldehyde and an unsaturated carboxylic acid in a satisfactory yield by the vapor-phase catalytic oxidation of a compound selected from propylene, isobutylene and tertiary butyl alcohol with molecular oxygen.

Furthermore, the catalyst for the production of methacrylic acid, which is made of the molding as above mentioned and contains a catalyst component made of a heteropolyoxide containing at least phosphorus and molybdenum, has the effect capable of producing methacrylic acid in a satisfactory yield by the vapor-phase catalytic oxidation of at least one kind of a compound selected from methacrolein, isobutylaldehyde, isobutane and isobutyric acid with molecular oxygen.

### BRIEF DESCRIPTION OF THE DRAWINGS

FIG. 1(a) is a schematic front view (it is noted that a side view and a rear view in this embodiment are the same as the front view) which shows one embodiment of the molding of the present invention, and FIG. 1(b) is a schematic plan view of the molding of FIG. 1(a) when it is viewed from its above.

FIG. 2(a) is a schematic sectional view taken along the X-X line of FIG. 1(b), and FIG. 2(b) is a schematic sectional view taken along the Y-Y line of FIG. 1(b).

FIG. 3 is a schematic front view (it is noted that a side view and a rear view in this embodiment are the same as the front view) which shows another embodiment of the molding of the present invention.

FIG. 4 (a) is a schematic enlarged sectional view showing one example of an extruding bore portion in an extrusion molding machine for the production of the molding of the present invention, and FIG. 4(b) is a schematic sectional view showing the extrusion molding machine of FIG. 4(a).

FIG. 5 is a graph for explaining operations which form a molding by repeating rotation and stopping of any one of the first and second dies using the extrusion molding machine shown in FIG. 4.

FIG. 6 is an explanatory view for explaining cutting positions of the molding extruded through an extrusion molding machine.

FIG. 7 (a) is a schematic enlarged sectional view showing another example of an extruding bore portion in an extrusion molding machine for the production of the molding of the present invention, and FIG. 7(b) is a schematic sectional view showing the extrusion molding machine of FIG. 7(a).

FIG. 8(a) is a schematic front view showing still further embodiment of the molding of the present invention, and FIG. 8(b) is a schematic plan view of the molding of FIG. 8(a) when it is viewed from its above.

FIG. 9 is a schematic enlarged view showing an extruding bore portion in an extrusion molding machine for the production of the molding shown in FIG. 8.

FIG. 10 is a graph for explaining operations which form a molding by repeating rotation and stopping of any one of the first and second dies using an extrusion molding machine which includes an extruding bore portion shown in FIG. 9.

FIG. 11(a) is a schematic plan view of catalysts produced in Comparative Example 2 or 3 when it is viewed from its above, and FIG. 11(b) is a schematic front view showing the catalyst of FIG. 11(a).

FIG. 12 is a schematic enlarged sectional view showing a still further example of an extruding bore portion in an extrusion molding machine for the production of the molding of the present invention.

FIG. 13 is a graph for explaining operations which form a molding by repeating rotation and stopping of any one of first and second dies using the extrusion molding machine shown in FIG. 12.

FIG. 14(a) is a schematic plan view showing a still further embodiment of the molding of the present invention when it is viewed from its above, and FIG. 14(b) is a schematic front view of the molding of FIG. 14(a).

FIG. 15 is a schematic enlarged sectional view showing a still further example of an extruding bore portion in an extrusion molding machine for the production of the molding of the present invention.

FIG. 16 is a graph for explaining operations which form a molding by repeating rotation and stopping of any one of first and second dies using the extrusion molding machine shown in FIG. 15.

FIG. 17(a) is a schematic plan view showing a still further embodiment in the molding of the present invention when it is viewed from its above, and FIG. 17(b) is a schematic front view of the molding of FIG. 17(a).

Description of Reference Numerals

| | |
|---|---|
| 10: | Molding, |
| 11: | Bridge portion, |
| 12: | Columnar portion |
| 13: | Through hole |
| 14: | Opening |
| 15: | Molding |
| 16: | Bridge portion |
| 17: | Columnar portion |
| 18: | Opening |
| 19: | Through hole |
| 20: | Extrusion molding machine |
| 21: | First die |
| 21a: | Groove of first die |
| 22: | Second die |
| 22a: | Groove of second die |
| 23: | Rotating unit |
| 23a: | Rotation axis |
| 23b: | Motor |
| 24: | Cutting unit |
| 25: | Flow path |
| 26: | First die |
| 26a: | Groove of first die |
| 27: | Second die |
| 27a: | Groove of second die |
| 28: | Molding |
| 29: | First die |
| 29a: | Groove of first die |
| 30: | Second die |
| 30a: | Groove of second die |
| 31: | Molding |
| 40: | Through hole |
| 41: | Molding |
| 42: | Columnar portion |
| 43: | Through hole |
| 44: | Bridge portion |
| 45: | Opening |
| 53: | Through hole |
| 54: | Opening |

### DETAILED DESCRIPTION OF THE INVENTION

### Molding

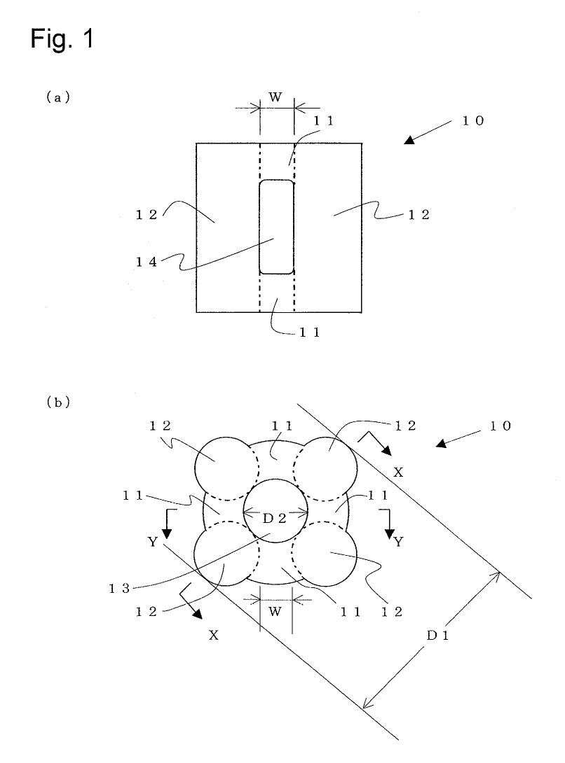

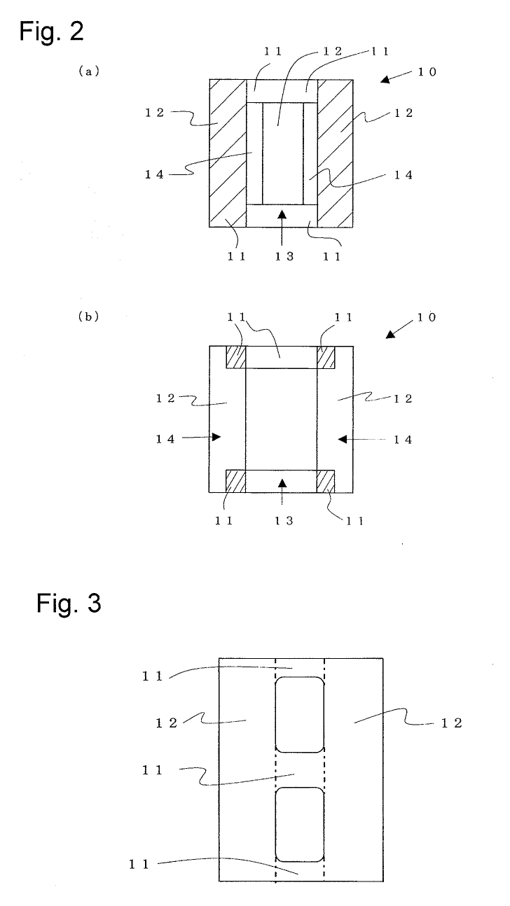

The molding according to the present invention will be described below with reference to the accompanying drawings. FIG. 1(a) is a schematic side view which shows one embodiment of the molding of the present invention, and FIG. 1(b) is a schematic plan view of the molding of FIG. 1(a). FIG. 2(a) is a schematic sectional view taken along the X-X line of FIG. 1(b), and FIG. 2(b) is a schematic sectional view taken along the Y-Y line of FIG. 1(b).

The molding of the present invention 10 shown in FIGS. 1(a) and 1(b) and FIGS. 2(a) and 2(b) shows a shape such that it includes a plurality of columnar portions (for example four columnar portions) 12 disposed with a predetermined gap between adjacent two columnar portions; bridge portions 11 each of which is provided at each ends in longitudinal directions of the plurality of columnar portions 12 and joins the adjacent columnar portions to each other; and a through hole 13 surrounded by the plurality of columnar portions 12 in the longitudinal directions of the columnar portions 12 (i.e. the extruding direction of the molding 10 described hereinafter) and openings 14 formed on a peripheral surface (i.e. the direction perpendicular to the extruding direction of the molding 10 described hereinafter) by a gap between the plurality of columnar portions 12.

In this embodiment, four columnar portions 12 are arranged at equal intervals between adjacent two columnar portions to form a square pillar and define a through hole 13 surrounded by the columnar portions. The bridge portions 11 are wound so as to cross all of the columnar portions 12 and the adjacent columnar portions 12 are joined to each other whereby, the bridge portions 12 substantially form a circular portion mentioned above. In other words, the circular portion is divided into the four bridge portions 11 by the four columnar portions 12. Between the adjacent columnar portions 12 and 12, an aperture 14 having a width corresponding to a gap therebetween is formed, and the bridge portions 11 are located above and under the opening 14 respectively.

The gap between columnar portions 12 and 12 as used herein, i.e. a width W of the opening 14 as shown is not particularly limited since it varies depending on the size of the molding, but is usually in the range from about 0.1 mm to 49 mm, and preferably from about 1 mm to 28 mm.

The cross-sectional shape of the columnar portion 12 is not limited to a circle and may be any shape. For example, it may be a semicircle, a triangle, a square or the like.

The cross-sectional shape of the bridge portion 11 is not particularly limited to a circle and may be any shape. For example, it may be a semicircle, a circle, a triangle, a square or the like. The size (or thickness) thereof is not particularly limited as long as it can join the adjacent columnar portions 12 and 12 to each other with a high strength when wound.

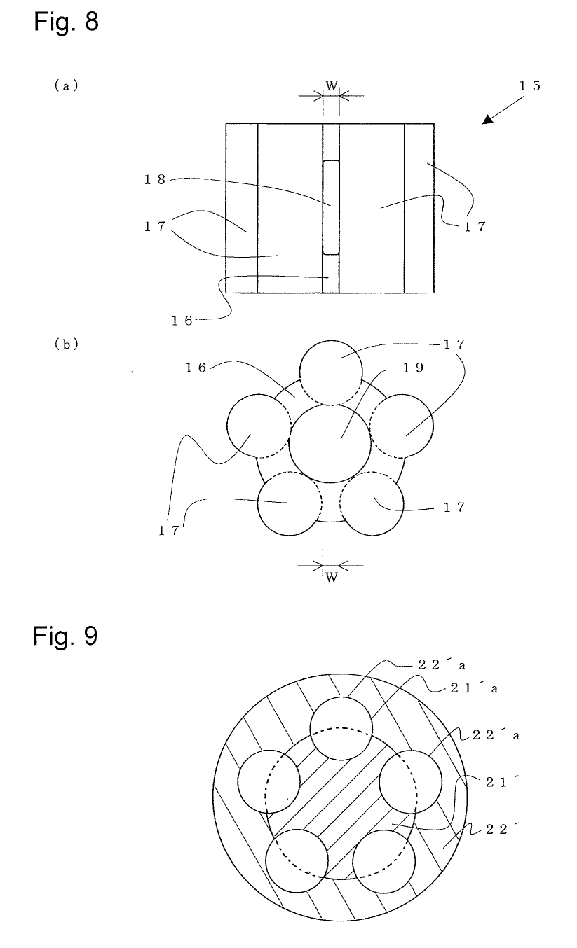

The number of the columnar portions 12 is not limited to four as shown in FIG. 1, and may be from three to nine. More preferably, the number is an odd number. For example, FIGS. 8(a) and 8(b) show other embodiment of the present invention in which five columnar portions 17 are arranged at equal intervals and are joined to each other at the bridge portions 16. Even in the case of such a molding, it is possible to form openings 18 on a peripheral surface and the through holes 19 on an upper and lower surfaces, respectively.

It is not absolutely necessary to form a gap between every adjacent two columnar portions 12. For example, the gap may be at least one, and the other columnar portions 12 may be joined to each other without a gap.

The length of the columnar portion 12 (i.e. the height of the molding 10) is from about 1 mm to 50 mm, and preferably from about 3 mm to 30 mm, and the diameter of the columnar portion 12 is from about 0.2 mm to 24 mm, and preferably from about 1 mm to 14 mm when taking the strength of the molding into consideration. The outer diameter D1 of the molding 10 is from about 1 mm to 50 mm, and preferably from about 3 mm to 30 mm, and the inner diameter of the molding 10 (i.e. the diameter D2 of the through hole 13) is from about 0.1 mm to 49 mm, and preferably from about 1 mm to 28 mm. It is noted that D2 is preferably from 90% to 10% of D1, and more preferably from 30% to 80% of D1.

In the case of the molding 10 shown in FIG. 1 and FIG. 2, although the columnar portions 12 are provided so that a portion of each columnar portion outwardly projects from the outer periphery of the bridge portion 11, the columnar portion may be provided so that a portion thereof inwardly projects from the inner periphery of the bridge portion 11.

In the case of the molding 10 shown in FIG. 1 and FIG. 2, although the bridge portions 11 are provided at both ends of the columnar portion 12, the bridge portions 11 may be provided at the center of the columnar portion 12, in addition to both ends, as shown in FIG. 3. In other words, it is also possible to provide the bridge portions 11 in the present invention in one or a plurality of stages at intervals in the longitudinal direction of the columnar portion 12.

As described above, the molding of the present invention has a feature in its shape, and therefore the kind and composition of a molding material constituting the molding are not particularly limited and may be appropriately selected according to an application of the molding.

For example, when the molding of the present invention is used as catalysts, it is possible to use a metal hydroxide such as aluminum hydroxide (gibbsite, bayerite, boehmite, pseudo-boehmite) and magnesium hydroxide; a metal oxide such as active alumina (χ-, κ-, γ-, δ-, ρ-, η-, pseudo γ-, θ-alumina, etc.); α-alumina; silica; titania (rutile, anatase, brookite); a zeolite; a complex metal oxide containing molybdenum, cobalt and bismuth as main components; a heteropoly acid comprising molybdenum, vanadium, phosphorus and the like; and the like.

When the molding of the present invention is used as catalyst carriers, it is possible to use a metal hydroxide such as cordierite, mullite, aluminum hydroxide (gibbsite, bayerite, boehmite, pseudo-boehmite) and magnesium hydroxide; a metal oxide such as active alumina (χ-, κ-, γ-, δ-, ρ-, η-, pseudo γ-, θ-alumina, etc.), α-alumina, silica, titania (rutile, anatase, brookite), zirconia and ceria; silica-alumina, magnesia spinel, calcia spinel, aluminum titanate, magnesium aluminum titanate, a zeolite, and the like.

When the molding of the present invention is used as adsorbents, desiccants and moisture absorbents, it is possible to use activated carbon, silica gel, active alumina (χ-, κ-, γ-, δ-, ρ-, η-, pseudo γ-, θ-alumina), silica-alumina, a zeolite, smectite, apatite and diatomaceous earth.

The molding of the present invention can also be formed by using, in addition to the above mentioned molding materials, various plastic materials.

The molding of the present invention can be used as the catalysts, the catalyst carriers, the adsorbents, the desiccants and the moisture absorbents. In particular, when the moldings of the present invention is used as the catalysts or the catalyst media for various catalyst reaction, it is preferred to use them while packing into a reactor such as a fixed bed reactor or other type vessel, in view of more effective utilization of the effects according to the present invention. In other words, since the moldings of the present invention can minimize the pressure drop even with being packed in any direction at random, and also has a high strength, it is possible to efficiently exhibit catalytic performances even if a reaction tube is packed with the moldings in a fixed bed reactor.

The molding of the present invention having the shape described above can be produced, for example, by the production method of the present invention which is described in detail below, but the method for producing a molding of the present invention is not limited thereto.

It is noted that the molding of the present invention can also be fired, if necessary, after forming by the below-described production method of the present invention.

### Method for Producing Molding

The molding of the present invention can be produced, for example, by an extrusion molding method in which a molding material is extruded using an extrusion molding machine including a first die having a plurality of grooves on its outer peripheral surface, and a ring-shaped or cylindrical second die fitted in the first die, having a plurality of grooves on its inner peripheral surface while repeating rotation and stopping of any one of the first and second dies. This extrusion molding method and the extrusion molding machine used in said method will be described in detail with reference to the accompanying drawings, but the molding method of the present invention is not limited to the method as a matter of course.

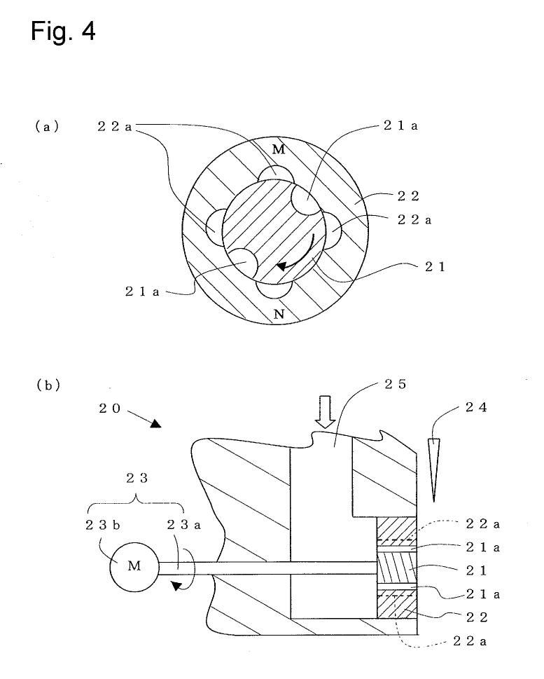

FIG. 4(a) is a schematic enlarged sectional view showing one example of the extruding bore portion in an extrusion molding machine for the production of a molding of the present invention, and FIG. 4(b) is a schematic sectional view showing the extrusion molding machine of FIG. 4(a).

The extrusion molding machine 20 shown in FIG. 4 includes a first die 21 having two grooves 21a on its outer peripheral surface, and a ring-shaped or cylindrical second die 22 fitted in the first die 21, having a plurality of grooves 22a on its inner peripheral surface. Specifically, both the first die 21 and the second die 22 are mounted onto a front surface of the extrusion molding machine 20 in the state where the first die 21 is fitted into the second die 22, so that a molding material is continuously extruded through grooves 21a of the first die 21 and grooves 22a of the second die 22.

There is no particular limitation as to dimensions of the first die 21 and the grooves 21a thereof, and also as to dimensions of the second die 22 and the grooves 22a thereof. For example, the outer diameter of the first die 21 is from about 0.3 mm to 48 mm, and preferably from about 2.0 mm to 29 mm, and the depth of the grooves 21a as R is from about 0.1 mm to 12 mm, and preferably from about 0.5 mm to 7 mm. The outer diameter of the second die 22 is from about 1 mm to 150 mm, and preferably from about 2 mm to 100 mm, and the inner diameter is from about 0.3 mm to 48 mm, and preferably from about 2.0 mm to 29 mm. The depth of the grooves 22a as R is from about 0.1 mm to 12 mm, and preferably from about 0.5 mm to 7 mm. Herein, R means a curvature radius (the same shall be applied hereinafter). It is noted that in the embodiment shown in FIG. 4, the number of the grooves 22a is four and that of the grooves 21a is two, but such numbers are not limited to such, and the numbers of the grooves 21a and the grooves 22a are appropriately selected, respectively, according to the number of the columnar portions 12 of the molding to be obtained.

The extrusion molding machine 20 further includes a rotation unit 23 for rotating the first die 21. This rotation unit 23 is not particularly limited and, for example, a conventional rotation unit such as a motor may be employed. Specifically, in the embodiment shown in FIG. 4, the first die 21 is rotated by rotationally driving a rotation axis 23a fixed to the first die 21 using a motor 23b. In this case, when each of the two grooves 21a of the first die 21 is joined together (or aligned) with any one of the four grooves 22a of the second die 22, the columnar portions 12 are formed of the molding material extruded through the respective grooves. When the two grooves 21a of the first die 21 shift from the four grooves 22a of the second die 22, the bridge portions 11 are formed of the molding material extruded only through the two grooves 21a of the first die 21.

To the contrary to the embodiment shown in FIG. 4, when the rotation unit 23 rotates the second die 22, the columnar portions 12 are formed of the molding material extruded through the grooves 21a of the first die 21 and the bridge portions 11 are formed of the molding material extruded through the grooves 22a of the second die 22. In the resultant molding, a portion of the columnar portion 12 protrudes inward on an inner peripheral surface (i.e. in the through hole 13) of the bridge portion 11.

The extruding operation of a molding material used for forming the molding 10 using the extrusion molding machine 20 as shown in FIG. 4 is carried out, for example, in the following sequences (i) to (iv):

(i) while extruding the molding material through grooves 21a and 22a of the first die 21 and second die 22, the first die 21 is rotated by 180 degrees from the position M where the grooves 21a and 22a are aligned with each other to the next position N where the grooves 21a and 22a of the first and second dies 21 and 22 are aligned with each other to form the bridge portions;

(ii) then, the rotation of the first and second dies 21 and 22 is stopped at the position N to form the columnar portions;

(iii) the first die 21 is rotated again by 180 degrees from the position N to the original position M to form the bridge portions; and

(iv) then, the rotation of the first and second dies 21 and 22 at the position M is stopped to form the columnar portions 12.

The above mentioned extruding operations are repeated to continuously form the molding 10.

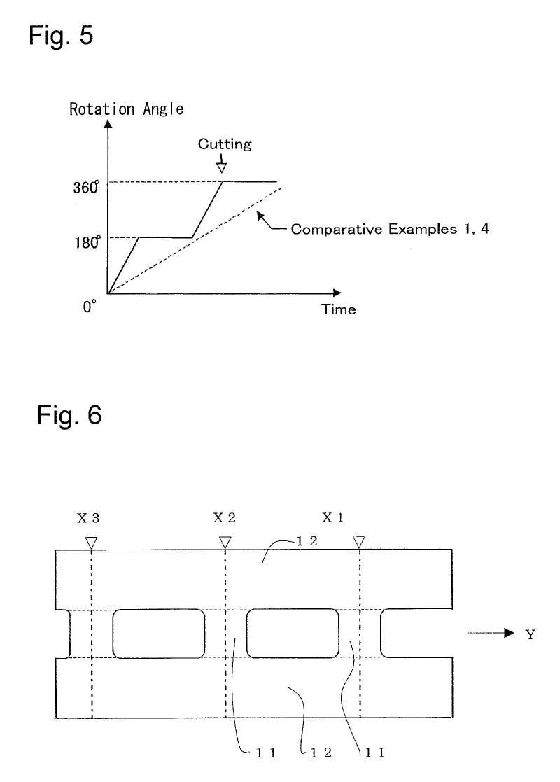

FIG. 5 shows a relationship between the molding time and the rotation angle of the first die 21. FIG. 5 also shows a relationship between the molding time and the rotation angle with respect to Comparative Examples 1 and 4 described hereinafter.

The above-described operations of rotation and stopping in the present invention can be carried out, for example, by sequential control. Herein, the rotation stopping time of the first die 21 can be adjusted according to the length of the aimed columnar portions 12.

When the bridge portions 11 are formed, the rotation speed of the first die 21 is important. When the rotation speed is lower than the extrusion rate of the molding material through the molding machine 20, the bridge portions 11 may form a spiral. Therefore, the rotation speed of the first die 21 is usually 2 times or more, and preferably from 4 times to 10 times as the extrusion rate of the molding material. The extrusion rate of the molding material is usually from 1 mm/min to 2,000 mm/min, and preferably from 10 mm/min to 1,000 mm/min. Such rotation speed is the same as that in the case of rotating the second die 22.

The extrusion molding machine 20 also includes a cutting unit 24 for cutting the molding material extruded through the first and second dies 21 and 22. The moldings 10 are continuously obtained by cutting thus extruded molding material into a predetermined length using the cutting unit 24.

There is no particular limitation on the cutting unit 24 and, for example, a conventionally known cutting unit such as a cutter knife or a wire rod (a piano wire, etc.) stretched across two guide rollers may be employed.

The cutting unit 24 may be driven by a motor so as to cross a front surface of an extruding bore of the dies 21 and 22, and preferably to cross a front surface of the dies 21 and 22 while being in contact with or in proximity to such surface.

Regarding the cutting position of the molding extruded continuously, for example, the molding 10 as shown in FIGS. 1 and 2 is cut at the positions X1, X2, X3, . . . where the bridge portion 11 is divided into two parts as shown in FIG. 6 so as to respectively form bridge portions 11 and 11 at both ends in the longitudinal direction of the columnar portions 12. The arrow Y indicates the extrusion direction of the molding. It is noted that when the molding as shown in FIG. 3 is to be obtained, the molding may be cut at the position X1 and then cut at the position X3.

The extrusion molding machine for the production of the molding in the present invention may be provided with a flow rate control valve (not shown) so as to control the molding speed of the molding material to be extruded through the grooves 21a and the grooves 22a.

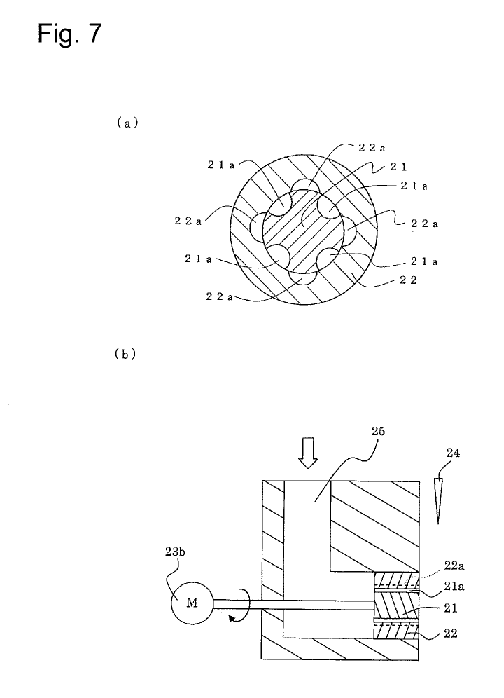

FIGS. 7(a) and 7(b) show another example of the method for producing the molding of the present invention. As shown in the drawings, in this embodiment, the grooves 21a of the first die 21 and the grooves 22a of the second die 22 are formed in the same number (four). Therefore, in the extruding operation, any one of first die 21 and the second die 22 in sequences (i) and (iii) may be rotated by 90 degrees. The other points are the same as in the above embodiment. It is noted that the same symbols or references are used in FIGS. 7(a) and 7(b) for the same constituent members as in FIGS. 4(a) and 4(b), and repetitive descriptions are omitted.

The molding thus obtained includes the plurality of columnar portions 12 disposed with a predetermined gap, and the bridge portions 11 each of which is provided at both ends in the longitudinal direction of the plurality of columnar portions 12 and joins the adjacent columnar portions to each other, and also includes through holes 13 surrounded by a plurality of columnar portions 12 in the longitudinal direction of the columnar portion 12, and openings 14 formed on a peripheral surface (i.e. a surface which is around the extrusion direction of the molding 10 described hereinafter) by gaps between the plurality of columnar portions 12.

Since the molding has a proper strength and a surface area which larger than that of a catalyst produced by the conventional production method when used as the catalyst, a pressure loss decreases when such moldings are packed into a fixed bed multi-tubular reactor or any other kind of vessel, and the molding has excellent catalytic activity.

The molding of the present invention can be preferably used not only as catalysts for the production of an unsaturated aldehyde and an unsaturated carboxylic acid and the production of methacrylic acid described below, but also as catalysts, catalyst precursors or catalyst carriers for the production of ethylene oxide, the production of propylene oxide, the production of 1,2-dichloroethane, the production of a synthetic gas, the production of hydrogen, reforming of a natural gas, reforming of kerosene, reforming of dimethylether, the production of dimethylether, dehydration of ethylbenzene, selective hydrogenation, oxidation, denitrification, hydrodesulfurization and the like.

### Catalyst for the Production of Unsaturated Aldehyde and Unsaturated Carboxylic Acid

### Production of Catalyst

The catalyst for the production of an unsaturated aldehyde and an unsaturated carboxylic acid according to the present invention is made of the molding including the plurality of columnar portions disposed with at least one gap; and the bridge portion which is disposed at least both ends of the adjacent columnar portions in the longitudinal direction of the plurality of columnar portions, and joins the adjacent columnar portions to each other; and also including the through holes surrounded by the plurality of columnar portions, and the openings formed on a peripheral surface by the gaps between the columnar portions; wherein a catalyst component is a complex oxide which contains molybdenum, bismuth and iron as indispensable components. This complex oxide may contain elements other than molybdenum, bismuth and iron and may contain, for example, nickel, cobalt, potassium rubidium, cesium, thallium and the like.

Preferred examples of the complex oxide can be represented by the following general formula (I):

MoₐBi_{b}Fe_{c}A_{d}BₑC_{f}D_{g}Oₓ (I)

wherein Mo, Bi and Fe represent molybdenum, bismuth and iron, respectively, A represents nickel and/or cobalt, B represents an element selected from manganese, zinc, calcium, magnesium, tin and lead, C represents an element selected from phosphorus, boron, arsenic, tellurium, tungsten, antimony, silicon, aluminum, titanium, zirconium and cerium, D represents an element selected from potassium, rubidium, cesium and thallium, 0<b≦10, 0<c≦10, 1≦d≦10, 1≦e≦10, 1≦f≦10 and 1≦g≦2 when a=12, and X is a value determined by the oxidation state of each element.

Among the complex oxides, those with the following compositions (excluding oxygen atom) are preferably used:

Mo₁₂Bi_{0.1-5}Fe_{0.5-5}Co₅₋₁₀Cs_{0.01-1} (I-1)

Mo₁₂Bi_{0.1-5}Fe_{0.5-5}Co₅₋₁₀Sb_{0.1-5}K_{0.01-1} (I-2)

Mo₁₂Bi_{0.1-5}Fe_{0.5-5}Ni₅₋₁₀Sb_{0.1-5}Si_{0.1-5}Tl_{0.01-1} (I-3)

As raw materials of the catalyst, compounds of the respective elements contained in the catalyst, for example, oxide, nitrate, sulfate, carbonate, hydroxide, oxo acid and an ammonium salt thereof, halide, and the like are usually used in the proportion satisfying a desired atomic ratio.

For example, molybdenum trioxide, molybdic acid, ammonium paramolybdate and the like can be used as a molybdenum compound, bismuth oxide, bismuth nitrate, bismuth sulfate and the like can be used as a bismuth compound, and iron(III) nitrate, iron(III) sulfate and iron(III) chloride, and the like can be used as an iron compound, respectively.

The catalyst precursor prepared from the above raw materials of the catalyst is fired (or calcined) under a molecular oxygen-containing gas, and then subjected to a heat treatment in the presence of a reducing substance.

This catalyst precursor can be usually prepared by mixing raw materials of the catalyst in water to obtain an aqueous solution or an aqueous slurry and drying the aqueous solution or the aqueous slurry.

The drying operation can be carried out, for example, by using a kneader, a box type dryer, a drum type dryer, a spray dryer, a flash dryer and the like.

The catalyst precursor obtained above is fired under an atmosphere of a molecular oxygen-containing gas. The concentration of molecular oxygen in this gas is usually from 1% to 30% by volume, and preferably from 10% to 25% by volume.

As a molecular oxygen source, air or pure oxygen is usually used and is optionally used as the molecular oxygen-containing gas after diluting with nitrogen, carbon dioxide, water, helium or argon.

The firing temperature is usually from 300° C. to 600° C., and preferably from 400° C. to 550° C. The firing time is usually from 5 minutes to 40 hours, and preferably from 1 hour to 20 hours.

In the present invention, the catalyst obtained by the above firing operation is subjected to a heat treatment in the presence of a reducing substance (hereinafter, the heat treatment in the presence of the reducing substance is sometimes simply referred to be a reduction treatment). It is possible to effectively improve activity of the catalyst by such a reduction treatment.

Examples of the reducing substance include hydrogen, ammonia, carbon monoxide, a hydrocarbon, an alcohol, an aldehyde, an amine and the like, and two or more kinds of these reducing substances can be optionally used. Herein, the hydrocarbon, the alcohol, the aldehyde and the amine preferably respectively have about 1 to 6 carbon atoms, and examples of the hydrocarbon include an aliphatic hydrocarbons such as methane, ethane, propane, n-butane, isobutane and the like; an unsaturated aliphatic hydrocarbons such as ethylene, propylene, α-butylene, β-butylene, isobutylene and the like; benzene and the like. Examples of the alcohol include a saturated aliphatic alcohols such as methyl alcohol, ethyl alcohol, n-propyl alcohol, isopropyl alcohol, n-butyl alcohol, isobutyl alcohol, secondary butyl alcohol, tertiary butyl alcohol and the like; an unsaturated aliphatic alcohols such as allyl alcohol, crotyl alcohol, metallyl alcohol and the like; phenol and the like.

Examples of the aldehyde include a saturated aliphatic aldehydes such as formaldehyde, acetaldehyde, propionaldehyde, n-butylaldehyde, isobutylaldehyde and the like; and an unsaturated aliphatic aldehydes such as acrolein, crotonaldehyde, methacrolein and the like. Examples of the amine include a saturated aliphatic amines such as methylamine, dimethylamine, trimethylamine, ethylamine, diethylamine, triethylamine and the like; an unsaturated aliphatic amines such as allylamine, diallylamine and the like; aniline and the like.

The reduction treatment is usually carried out by subjecting the catalyst to a heat treatment under an atmosphere of a gas containing the above reducing substance.

The concentration of the reducing substance in this gas is usually from 0.1% to 50% by volume, preferably from 1% to 50% by volume, and more preferably from 3% to 30% by volume, and the reducing substance may be diluted with nitrogen, carbon dioxide, water, helium, argon or the like so as to adjust to the concentration within the above range. The molecular oxygen may be allowed to exist as long as the effects of the reduction treatment are not impaired, but may be usually absent.

The temperature of the reduction treatment is usually from 200° C. to 600° C., and preferably from 300° C. to 500° C. The time of the reduction treatment is usually from 5 minutes to 20 hours, and preferably from 30 minutes to 10 hours.

The reduction treatment is preferably carried out while passing a gas containing a reducing substance through vessel such as a tube or a box after charging the catalyst in the vessel. During the reduction treatment, the gas discharged from the vessel may also be recycled.

It is also possible that after a reaction tube for vapor-phase catalytic oxidation is packed with the catalyst and the reduction treatment of the catalysts is carried out by passing a gas containing the reducing substance through the reaction tube, the vapor-phase catalytic oxidation is subsequently carried out.

Mass loss of the catalyst is usually observed through the reduction treatment and it is considered that such mass loss arises since the catalyst loses its lattice oxygen. The mass loss ratio is preferably from 0.05% to 6%, and more preferably from 0.1% to 5%. When the reduction excessively proceeds and thus the mass loss excessively increases, the catalytic activity sometimes deteriorates, on the contrary. In this case, the mass loss ratio may be decreased by firing the molecular oxygen-containing gas again under the atmosphere of the molecular oxygen-containing gas. It is noted that the mass loss ratio is determined by the following equation.

Mass loss ratio(%)=[(mass of catalyst before reduction treatment−mass of catalyst after reduction treatment)/(mass of catalyst before reduction treatment)]×100

In the case of the reduction treatment, a reducing substance per se and/or a decomposition product derived from the reducing substance sometimes remain on/in the catalyst after the reduction treatment depending on the kind of the reducing substance to be used and heat treatment conditions. In such remaining happens, after measuring the mass of the remaining substances on/in the catalyst, the mass after the reduction treatment may be calculated by subtracting the mass of the remaining substances from the mass of the catalyst containing the remaining substances. Since a main substance of the remaining substances is typically carbon, for example, the mass may be determined by measuring am amount of total carbon (TC).

It is noted that the catalyst of the present invention may be molded at the stage of a catalyst precursor or after firing the catalyst precursor, or after carrying out the reduction treatment of the catalyst precursor.

### Production of Unsaturated Aldehyde and Unsaturated Carboxylic Acid

Acrolein and acrylic acid can be produced in a good yield by the vapor-phase catalytic oxidation of propylene with molecular oxygen using the above mentioned catalyst. Also, methacrolein and methacrylic acid can be produced in a good yield by the vapor-phase catalytic oxidation of isobutylene and/or tertiary butyl alcohol with molecular oxygen.

The vapor-phase catalytic oxidation reaction is usually carried out by packing a fixed bed multi-tubular reactor with a catalyst and feeding a raw gas containing a raw compound selected from propylene, isobutylene and tertiary butyl alcohol, and molecular oxygen. Also the reaction can be carried out in a fluidized bed or a moving bed.

Air is usually used as a molecular oxygen source, and the raw gas contains, as components other than the raw compound and the molecular oxygen, nitrogen, carbon dioxide, carbon monoxide, steam and/or the like.

The reaction temperature is usually from 250° C. to 400° C., and the reaction pressure may be a reduced pressure but is usually from 100 kPa to 500 kPa. The amount of the molecular oxygen is usually from 1 to 3 mol per mol of the raw compound. The space velocity SV of the raw gas is usually from 500/hour to 5,000/hour in terms of the standard temperature pressure (STP).

### Catalyst Made of Heteropolyoxide for the Production of Methacrylic Acid

### Production of Catalyst

The catalyst for the production of methacrylic acid according to the present invention is a catalyst made of a molding which includes a plurality of columnar portions disposed with at least one gap; and bridge portions each of which is disposed at least both ends in longitudinal directions of a plurality of adjacent columnar portions, and joins the adjacent columnar portions to each other; and which molding also includes through holes surrounded by the plurality of columnar portions, and openings formed on a peripheral surface of the molding by a gap between the adjacent columnar portions; wherein a catalyst component is made of a heteropoly acid compound containing at least phosphorus and molybdenum, and the heteropoly acid compound may also be a free heteropoly acid or a salt of a heteropoly acid. The catalyst component is preferably made of an acidic salt (partially neutralized salt) of the heteropoly acid, and more preferably made of an acidic salt of Keggin type heteropoly acid.

More preferably, the above mentioned heteropoly acid compound of the catalyst further contains vanadium, at least one kind of an element selected from potassium, rubidium, cesium and thallium (which is hereinafter sometimes referred to as an X element) and at least one kind of an element selected from copper, arsenic, antimony, boron, silver, bismuth, iron, cobalt, zinc, lanthanum and cerium (which is hereinafter sometimes referred to as a Y element). Usually, a catalyst containing 3 atoms or less of each phosphorus, vanadium, the X element and the Y element based on 12 molybdenum atoms is preferably used.

As the raw material of the above mentioned catalyst, compounds containing the respective elements which are to be contained in the catalyst, for example, an oxo acid, an oxolate, an oxide, a nitrate, a carbonate, a hydroxide, a halide and the like of the respective elements are usually used in amounts which satisfy a desired atomic ratio.

For example, a phosphoric acid, a phosphate or the like is used as a compound containing phosphorus; a molybdic acid, a molybdate, a molybdenum oxide, a molybdenum chloride or the like is used as a compound containing molybdenum; and a vanadic acid, a vanadate, a vanadium oxide, a vanadium chloride or the like is used as a compound containing vanadium. Furthermore, an oxide, a nitrate, a carbonate, a hydroxide, a halide or the like is used as a compound containing the X element, and an oxo acid, an oxolate, nitrate, a carbonate, a hydroxide, a halide or the like is used as a compound containing the Y element.

In the present invention, the raw materials of the catalyst are mixed in water so as to obtain an aqueous mixture containing the raw materials of the catalyst, and after drying the aqueous mixture, a first-stage firing is carried out under an oxidizing gas atmosphere at a predetermined temperature. Such a drying operation is preferably carried out by spray drying using a spray dryer. After such drying, the resultant dried product may be molded into a predetermined shape as described hereinafter, followed by a first-stage firing. Alternatively, after such drying, the dried product may be subjected to a heat treatment (pre-firing), followed by molding and further first-stage firing. Alternatively, after drying, the dried product may be molded, followed by a heat treatment (pre-firing) and further first-stage firing. Upon carrying out such molding, the dried product may be molded into a columnar shape, a spherical shape, a ring-shape or the like using a molding aid if necessary. When the dried product is subjected to the heat treatment (pre-firing), such heat treatment is preferably carried out under an atmosphere of an oxidizing gas or a non-oxidizing gas at a temperature of about 180° C. to 300° C.

It is effective when an aqueous mixture containing an ammonium radical (or ion) is obtained by using an ammonium compound as the raw material of the catalyst and/or adding ammonia and/or an ammonium salt as the raw material, the obtained aqueous mixture is molded after subjecting to the heat treatment, or the obtained aqueous mixture is subjected to the heat treatment after molding. According to these preparations, it is possible to form a structure of a Keggin type heteropoly acid salt during the heat treatment, and the Keggin type heteropoly acid salt thus obtained becomes particularly preferred subject to be fired according to the present invention.

In the present invention, after the above drying operation, a first-stage firing operation is carried out under an atmosphere of an oxidizing gas at a predetermined temperature, then the temperature is raised to a predetermined temperature under an atmosphere of a non-oxidizing gas containing a predetermined amount of water, and then a second-stage firing operation is carried out under an atmosphere of a non-oxidizing gas at a predetermined temperature. It is possible to produce a catalyst for the production of methacrylic acid in a satisfactory yield of methacrylic acid with an excellent catalyst lifetime by carrying out a series of the above mentioned molding, firing, temperature raising and firing operations.

The oxidizing gas used in the first-stage firing operation is a gas containing an oxidizing substance and is typically an oxygen-containing gas, and the oxygen concentration is usually from about 1% to 30% by volume. Air and pure oxygen are usually used as the oxygen source and are optionally diluted with an inert gas. The oxidizing gas used in the first-stage firing operation may optionally contain 0.1% to 10% by volume of moisture, and preferably 0.5% to 5% by volume of moisture.

The temperature of the first-stage firing operation is from 300° C. to 400° C., and preferably from 360° C. to 400° C. When the temperature of the first-stage firing is lower than 300° C., the resultant catalyst sometimes shows insufficient activity. In contrast, when the temperature is higher than 400° C., since the catalyst is likely to be decomposed and sintered, the resultant catalyst sometimes shows insufficient activity.

After the first-stage firing operation, the temperature is raised to 420° C. or higher under an atmosphere of a non-oxidizing gas containing a predetermined amount of water. The non-oxidizing gas as used herein is a gas which does not substantially contain an oxidizing substance such as oxygen or the like, and examples thereof include inert gases such as nitrogen, carbon dioxide, helium, argon and the like. The content of water in the non-oxidizing gas is from 0.1% to 10% by volume, and preferably from 0.5% to 5% by volume. When the content is less than 0.1% by volume, the resultant catalyst sometimes shows insufficient activity.

After the temperature rising operation, a second-stage firing operation is carried out under an atmosphere of a non-oxidizing gas at a predetermined temperature. The temperature of the second-stage firing operation is from 400° C. to 500° C., and preferably from 420° C. to 450° C. When the temperature of the second-stage firing is lower than 400° C., the resultant catalyst sometimes shows insufficient activity. In contrast, when the temperature is higher than 500° C., since the catalyst is likely to be decomposed and sintered, the resultant catalyst sometimes shows insufficient activity.

The non-oxidizing gas used in the second-stage firing operation is a gas which does not substantially contain an oxidizing substance such as oxygen, similarly to the above, and the non-oxidizing gas used in the second-stage firing may contain water or needs contain no water. When water is contained, the content of water is usually from 0.1% to 10% by volume, and preferably from 0.5% to 5% by volume.

Each of firing times is appropriately adjusted and is usually from about 1 hour to 20 hours. The temperature raising time is usually from about 0.5 hours to 10 hours. It is preferred to carry out the firing operations or the temperature raising operation while flowing, as an atmosphere gas upon such operation, a gas which is to be used in such operation. The sequence of the first-stage firing operation using the oxidizing gas and the second-stage firing using the non-oxidizing gas may be reversed.

### Production of Methacrylic Acid

Methacrylic acid can be produced in a satisfactory yield by vapor-phase catalytic oxidation of at least one kind of a compound selected from methacrolein, isobutylaldehyde, isobutane and isobutyric acid with molecular oxygen using the catalyst made of the heteropoly acid.

Methacrylic acid is usually produced by packing a fixed bed polycyclic reactor with the above mentioned catalyst and feeding a raw gas containing a raw compound and oxygen into the fixed bed reactor. Also, the reaction can be carried out in a fluidized bed or a moving bed. Air is usually used as an oxygen source, and the raw gas may contain, as components other than the raw compound and oxygen, nitrogen, carbon dioxide, carbon monoxide, steam and the like.

When methacrolein is used as the raw material, the reaction is usually carried out under the conditions of the concentration of methacrolein in a raw gas of 1% to 10% by volume, the molar ratio of oxygen to methacrolein of 1 to 5, the space velocity of 500/hour to 5000/hour (STP basis), the reaction temperature of 250° C. to 350° C. and the reaction pressure of 0.1 MPa to 0.3 MPa. Methacrolein as the raw material needs not to be a high purity product, and for example, a reaction product gas containing methacrolein can also be used which gas is obtained by the vapor phase catalytic reaction of isobutylene.

Also, when isobutene is used as the raw material, the reaction is usually carried out under the conditions of the concentration of isobutane in the raw gas of 1% to 85% by volume, the steam concentration in the raw gas of 3% to 30% by volume, the molar ratio of oxygen to isobutene of 0.05 to 4, the space velocity of 400/h to 5000/h (STP basis), the reaction rate of 250° C. to 400° C. and the reaction pressure of 0.1 MPa to 1 MPa. When isobutyric acid or isobutylaldehyde are used as the raw material, neatly the same reaction conditions as those used when methacrolein is used as the raw material are employed.

### Aluminum Titanate-Based Crystal Molding

The molding of the present invention is a molding which includes a plurality of columnar portions disposed with at least one gap; and a bridge portion which is disposed at least both ends in longitudinal directions of the pl plurality of ural columnar portions, and joins adjacent columnar portions to each other; and also which includes through holes surrounded by the plurality of columnar portions, and openings formed on a peripheral surface by a gap between the adjacent columnar portions; the molding containing an aluminum titanate crystal.

In the present invention, the molding containing the aluminum titanate-based crystal is produced by firing a molding of a raw mixture which contains an aluminum source powder and a titanium source powder, and the raw mixture may further contain a magnesium source powder and a silicon source powder. The phrase “containing an aluminum titanate-based crystal” means that an aluminum titanate-based crystal phase exists in a crystal phase constituting the molding, and the aluminum titanate-based crystal phase may be, for example, an aluminum titanate crystal phase, a magnesium aluminum titanate crystal phase and/or the like, and also may contain other crystal phases.

The above mentioned molding contains at least titanium and aluminum elements and sometimes contains, in addition to these elements, magnesium and silicon. Furthermore, the molding may contain elements other than titanium, aluminum, magnesium and silicon and also may contain, for example, zirconium, tungsten, cerium, sodium, iron and the like.

### Aluminum Source Powder

The aluminum source powder contained in the raw mixture used in the present invention is a powder of a compound which contains aluminum element constituting the molding. Examples of the aluminum source powder include a powder of an alumina (aluminum oxide, Al₂O₃). The crystal form of alumina includes, for example, γ type, pseudo γ type, δ type, θ type, α type, ρ type, η type, χ type and κ type, and also may be amorphous.

The aluminum source powder used in the present invention may be a power of a compound which is converted into alumina by firing alone in air. Examples of such compound include an aluminum salt, an aluminum alkoxide, an aluminum hydroxide, metallic aluminum and the like.

The aluminum salt may be an aluminum inorganic salt with an inorganic acid, or an aluminum organic salt with an organic acid.

Specific examples of the aluminum inorganic salt include aluminum nitrates such as aluminum nitrate, aluminum ammonium nitrate and the like; aluminum carbonates such as aluminum ammonium carbonate and the like; aluminum chlorides and the like.

Specific examples of the aluminum organic salt include aluminum oxalate, aluminum acetate, aluminum stearate, aluminum lactate, aluminum laurate and the like.

Specific examples of the aluminum alkoxide include aluminum isopropoxide, aluminum ethoxide, aluminum sec-butoxide, aluminum tert-butoxide and the like.

The aluminum hydroxide may be, for example, an aluminum hydroxide with a crystal type such as gibbsite type, bayerite type, norstrandite type, boehmite type or pseudo boehmite type, and also may be an amorphous aluminum hydroxide.

The amorphous aluminum hydroxide also includes, for example, aluminum hydrolyzate obtained by hydrolyzing an aqueous solution of a water-soluble aluminum compound such as an aluminum salt, an aluminum alkoxide or the like.

In the present invention, as the aluminum source powder, one kind thereof may be used alone, and also two or more kinds thereof may be used in combination.

Among these powders, an alumina powder is preferably used as the aluminum source powder. The aluminum source powder can contain a trace component which is derived from the raw material thereof or inevitably contained in the manufacturing process.

There is no particular limitation on the particle diameter of the aluminum source powder. Usually, it is possible to use an aluminum source powder having a particle diameter corresponding to a volume-based cumulative percentage of 50% (D50), measured by the laser diffraction method, within a range from 0.1 μm to 100 μm, and preferably from 1 μm to 60 μm. When the particle diameter of the aluminum source powder is more than 100 μm, for example, water holding capacity of the aluminum source powder deteriorates in wet molding such as granulation or extrusion, and thus it becomes difficult to mold. In contrast, when the particle diameter is less than 0.1 μm, the powder is likely to float in a vapor phase, and thus it becomes difficult to handle the powder.

The aluminum source powder used in the present invention may have a single-modal particle diameter distribution, a bi-modal particle diameter distribution, or a particle diameter peak more than that described above as long as the aluminum source powder satisfies the above mentioned range of the particle diameter.

As the aluminum source powder which satisfies the above mentioned range of the particle diameter, a commercially available product may be used as it is, or an aluminum source powder satisfying the above mentioned range of the particle diameter may be obtained by subjecting a commercially available aluminum source powder to a treatment such as comminution, cracking, classification, screening, granulation or the like.

### Titanium Source Powder

The titanium source powder contained in the raw mixture is a powder of a compound which contains a titanium element constituting the molding and the compound includes, for example, a powder of titanium oxide.

Examples of the titanium oxide include titanium(IV) oxide, titanium(III) oxide, titanium(II) oxide and the like. Among these titanium oxides, titanium(IV) oxide is preferably used.

Examples of the titanium(IV) oxide include titanium(IV) oxides with crystal types such as anatase type, rutile type, brookite type and the like, and the titanium(IV) oxide may also be amorphous titanium(IV) oxide. Among these titanium(IV) oxides, anatase or rutile type titanium(IV) is more preferably.

The titanium source powder used in the present invention may also be a powder of a compound which is converted into titania (titanium oxide) by firing alone in air.

Examples of such compound include a titanium salt, a titanium alkoxide, a titanium hydroxide, a titanium nitride, a titanium sulfide, metallic titanium and the like.

Specific examples of the titanium salt include titanium trichloride, titanium tetrachloride, titanium(IV) sulfide, titanium(IV) sulfate and the like.

Specific examples of the titanium alkoxide include titanium(IV) ethoxide, titanium(IV) methoxide, titanium(IV) t-butoxide, titanium(IV) isobutoxide, titanium(IV) n-propoxide, titanium(IV) tetraisopropoxide, chelete compounds thereof and the like.

In the present invention, as the titanium source powder, one kind thereof may be used alone, and two or more kinds thereof may be used in combination.

Among the titanium source powders, the titanium oxide powder is preferably used, and titanium(IV) oxide powder is more preferably used. The titanium source powder can contain a trace component which is derived from the raw material thereof or inevitably contained in the manufacturing process.

There is no particular limitation on the particle diameter of the titanium source powder. Usually, it is possible to use a titanium source powder having a particle diameter corresponding to a volume-based cumulative percentage of 50% (D50), measured by the laser diffraction method, within a range from 0.5 μm to 25 μm. It is preferred to use a titanium source powder having D50 within a range from 1 μm to 20 μm. And thus, it is possible to effectively suppress nucleus of aluminum titanate generated at random during firing so as to form more homogeneous structure of a aluminum titanate-based crystal. Formation of more homogeneous structure of the aluminum titanate-based crystal contributes to reduce scatter in heat resistance and mechanical strength. The titanium source powder sometimes exhibits a bi-modal particle diameter distribution. When using the titanium source powder which exhibits the bi-modal particle diameter distribution, the particle diameter of particles which form a peak at a larger particle diameter, measured by the laser diffraction method, is preferably within a range from 20 μm to 50 μm.

The mode diameter measured by the laser diffraction method of the titanium source powder is not particularly limited and may be within a range from 0.3 μm to 60 μm.

A molar ratio of the content of the aluminum source powder in terms of Al₂O₃ (alumina) to that of the titanium source powder in terms of TiO₂ (titania) in the raw mixture is preferably adjusted within a range from 35:65 to 45:55, and more preferably from 40:60 to 45:55. It is advantageous that the reaction of conversion into aluminum titanate rapidly proceeds when the titanium source powder is excessively used relative to the aluminum source powder within the above range.

### Magnesium Source Powder

The raw mixture may contain a magnesium source powder. The magnesium source powder is a powder of a compound which contains a magnesium element constituting the molding, and such powder includes, for example, in addition to a powder of magnesia (magnesium oxide, MgO), a powder of a compound which is converted into magnesia by firing in air. Examples of the latter include a magnesium salt, a magnesium alkoxide, a magnesium hydroxide, a magnesium nitride, metallic magnesium and the like.

Specific examples of the magnesium salt include magnesium chloride, magnesium perchloride, magnesium phosphate, magnesium pyrophosphate, magnesium oxalate, magnesium nitrate, magnesium carbonate, magnesium acetate, magnesium sulfate, magnesium citrate, magnesium lactate, magnesium stearate, magnesium salicylate, magnesium myristate, magnesium gluconate, magnesium dimethacrylate, magnesium benzoate and the like.

Specific examples of the magnesium alkoxide include magnesium methoxide, magnesium ethoxide and the like.

When the raw mixture contains the aluminum source powder, the titanium source powder and the magnesium source powder, a molar ratio of the aluminum source powder in terms of Al₂O₃ (alumina) to the titanium source powder in terms of TiO₂ (titania) in the raw mixture is preferably adjusted within a range from 35:65 to 45:55, and more preferably from 40:60 to 45:55.

It is also possible to use, as the magnesium source powder, a powder of a compound which serves both as a magnesium source and as an aluminum source. Examples of such compound include magnesia spinel (MgAl₂O₄). When the powder of the compound, which serves both as a magnesium source and as an aluminum source, is used as the magnesium source powder, a molar ratio of the total of the aluminum source powder in terms of Al₂O₃ (alumina) in the raw mixture and the Al component in terms of Al₂O₃ (alumina) contained in the powder of the compound which serves both as a magnesium source and as an aluminum source to the titanium source powder in terms of TiO₂ (titania) is preferably adjusted within a range from 35:65 to 45:55, and more preferably from 40:60 to 45:55.

In the present invention, as the magnesium source powder, one kind thereof may be used alone, and two or more kinds thereof may be used in combination. The magnesium source powder can contain a trace component which is derived from the raw material thereof or inevitably contained in the manufacturing process.

There is no particularly limitation on the particle diameter of the magnesium source powder. Usually, it is possible to use a magnesium source powder having a particle diameter corresponding to a volume-based cumulative percentage of 50% (D50), measured by a laser diffraction method, within a range from 0.5 μm to 30 μm. D50 of the magnesium source powder is preferably within a range from 3 μm to 20 μm, and thus more homogeneous structure of a magnesium aluminum titanate-based crystal can be formed. Formation of the homogeneous structure contributes to reduction of unevenness in heat resistance and mechanical strength.

A molar ratio of the content of the magnesium source powder in terms of MgO (magnesia) in the raw mixture to the total of the amount of the aluminum source powder in terms of Al₂O₃ (alumina) and that of the titanium source powder in terms of TiO₂ (titania) is preferably from 0.03 to 0.15, and more preferably from 0.03 to 0.12. It is possible to improve mechanical strength and heat resistance of the molding by adjusting the content of the magnesium source powder within the above range.

### Silicon Source Powder

The silicon source powder contained in the raw mixture is a powder of a compound which forms a silicic acid glass phase composed mainly of an aluminum titanate-based crystal to be converted into a composite. It is possible to improve heat resistance of the molding by mixing the molding with the silicic acid glass phase. Examples of the silicon source powder include powders of silicon oxides (silica) such as silicon dioxide, silicon monoxide and the like.

The silicon source powder may be a powder of a compound which is converted into silica (SiO₂) by firing in air.

Examples of such compound include silicic acid, silicon carbide, silicon nitride, silicon sulfide, silicon tetrachloride, silicon acetate, sodium silicate, sodium orthosilicate, silicone resin, feldspar, glass frit, glass fiber and the like. Among these compounds, the feldspar and the glass frit are preferably used, and the glass frit is more preferably used in view of ease of the industrial availability and stable composition. The glass frit means flaky or powdered glass obtained by comminution of glass. It is also preferred to use, as the silicon source powder, a powder made of a mixture of the feldspar and the glass frit.

When the glass frit is used, those having a yield (or deformation) point of 700° C. or higher are preferably used in view of further improvement of heat resistance of the resultant molding. In the present invention, the yield point of the glass frit is defined as the temperature (° C.) at which the expansion of the glass frit stops and then the shrinkage thereof starts when measuring expansion of the glass frit from a low temperature using Thermo Mechanical Analyzer (TMA).

It is possible to use, as the glass which forms the glass frit, common silicic acid glass containing silicic acid (SiO₂) as a main component (50% by mass or more in all components). Similarly to the common silicic acid glass, the glass which forms the glass frit may contain, as other components, alumina (Al₂O₃), sodium oxide (Na₂O), potassium oxide (K₂O), calcium oxide (CaO), magnesia (MgO), and the like. The glass which forms the glass frit may contain ZrO₂ so as to improve hot water resistance of the glass per se.

In the present invention, as the silicon source powder, one kind thereof may be used alone, and two or more kinds thereof may be used in combination. The silicon source powder can contain a trace component which is derived from the raw material or inevitably contained in the manufacturing process.

There is no particular limitation on the particle diameter of the silicon source powder. Usually, it is possible to use a silicon source powder having a particle diameter corresponding to a volume-based cumulative percentage of 50% (D50), measured by the laser diffraction method, within a range from 0.5 μm to 30 μm. A silicon source powder having D50 within a range from 1 μm to 20 μm is preferably used, and thus the filling ratio of the molding of the raw mixture can be improved to obtain a fired body having higher mechanical strength and heat resistance.

When the raw mixture contains the aluminum source powder, the titanium source powder and the silicon source powder, a molar ratio of the aluminum source powder in terms of Al₂O₃ (alumina) to the titanium source powder in terms of TiO₂ (titania) in the raw mixture is preferably adjusted within a range from 35:65 to 45:55, and more preferably from 40:60 to 45:55. A molar ratio of the content of the magnesium source powder in terms of MgO (magnesia) in the raw mixture to the total of the aluminum source powder in terms of Al₂O₃ (alumina) and the titanium source powder in terms of TiO₂ (titania) is preferably adjusted within a range from 0.03 to 0.15, and more preferably from 0.03 to 0.12.

In the present invention, the content of the silicon source powder in inorganic components contained in the raw mixture is adjusted to 5% by mass or less, and preferably 4% by mass or less, so as to obtain a molding having satisfactory mechanical strength and heat resistance. The content of the silicon source powder in the inorganic components contained in the raw mixture is preferably adjusted to 2% by mass or more. The inorganic components contained in the raw mixture are compounds which contain elements which constitute the molding, and are typically the aluminum source powder, the titanium source powder, the magnesium source powder and the silicon source powder. When additives (such as pore-forming agents, binders, lubricants, plasticizers, dispersing agents, etc.) contained in the raw mixture contain inorganic components, such inorganic components are also included. When the content of the silicon source powder in the inorganic components contained in the raw mixture is more than 5% by mass or less than 2% by mass, satisfactory mechanical strength and heat resistance may not be obtained.

When the raw mixture contains the aluminum source powder, the titanium source powder, the magnesium source powder and the silicon source powder, a molar ratio of the aluminum source powder in terms of Al₂O₃ (alumina) and the titanium source powder in terms of TiO₂ (titania) in the raw mixture is preferably adjusted within a range from 35:65 to 45:55, and more preferably from 40:60 to 45:55. A molar ratio of the content of the magnesium source powder in terms of MgO (magnesia) in the raw mixture to the total of the aluminum source powder in terms of Al₂O₃ (alumina) and the titanium source powder in terms of TiO₂ (titania) is preferably adjusted within a range from 0.03 to 0.15, and more preferably from 0.03 to 0.12.

In the present invention, similarly to the complex oxide such as the above mentioned magnesia spinel (MgAl₂O₄), a compound containing two or more metallic elements as components thereof among titanium, aluminum, silicon and magnesium can be used as the raw powder. In this case, it can be considered that such compound is the same as a mixture obtained by mixing the respective metal source compounds. Based on such consideration, each content of the aluminum source material, the titanium source material, the magnesium source material and the silicon source material is adjusted so that a molar ratio of the aluminum source powder in terms of Al₂O₃ to the titanium source powder in terms of TiO₂ in the raw mixture is within a range from 35:65 to 45:55, and a molar ratio of the magnesium source powder in terms of MgO to the total of the aluminum source powder in terms of Al₂O₃ and the titanium source powder in terms of TiO₂ in the raw mixture is within a range from 0.03 to 0.15.

The raw mixture may contain aluminum titanate and magnesium aluminum titanate per se and, for example, when magnesium aluminum titanate is used as a constituent component of the raw mixture, magnesium aluminum titanate corresponds to a raw material which serves as a titanium source, as an aluminum source and as a magnesium source.

### Pore-Forming Agent

The raw mixture can contain a pore-forming agent. In the present invention, there is no particular limitation on the particle diameter of the pore-forming agent. Usually, it is possible to use a pore-forming agent having a particle diameter corresponding to a volume-based cumulative percentage of 50% (D50), measured by the laser diffraction method, within a range from 10 μm to 50 μm.

There is no particular limitation on the kind of the pore-forming agent (constituent material), and examples thereof include resins such as a polyethylene, a polypropylene, a polymethyl methacrylate and the like, and hollow particles of these resins; a water-absorbing resins such as a partial sodium salt of a crosslinked acrylic acid polymer, a modified polyalkylene oxide, a crosslinked isobutylene-maleic anhydride copolymer and the like; plant-based materials such as starch, nuts shell, walnuts shell, corn, corn starch and the like; carbon materials such as graphite and the like. The pore-forming agent may be one which can serve as an inorganic component contained in the raw mixture, and examples thereof include alumina hollow beads, titania hollow beads, hollow glass particles and the like. As the pore-forming agent, a commercially available product can be used as it is, or those obtained by appropriately screening the commercially available product may be used.

The content of the pore-forming agent contained in the raw mixture is usually from 0.1 parts to 50 parts by mass, and preferably from 0.2 parts to 25 parts by mass, based on the total amount (100 parts by mass) of the aluminum source powder, the titanium source powder, the magnesium source powder and the silicon source powder. When the content of pore-forming agent is less than 0.1 parts by mass, pores are not formed and thus the effect of adding the pore-forming agent cannot be obtained. In contrast, when the content of pore-forming agent is more than 50 parts by mass, the strength of the resultant molding decreases.

In the present invention, the raw mixture containing the aluminum source powder, the titanium source powder, the magnesium source powder, the silicon source powder, and the pore-forming agent used optionally are molded to obtain a molding, and then the molding is fired so as to obtain a molding containing a magnesium aluminum titanate-based crystal.

As the machine which is used for molding the raw mixture, for example, an extrusion molding machine is used. When the extrusion molding of the raw mixture is carried out, for example an additive may be added, in addition to the pore-forming agent, to the raw mixture. Such additive includes a binder, a lubricant agent and plasticizer, a dispersing agent, a solvent and the like.

The above mentioned binder includes celluloses such as methyl cellulose, carboxymethyl cellulose, sodium carboxymethyl cellulose and the like; alcohols such as a polyvinyl alcohol and the like; salts such as lignine sulfonate and the like; waxes such as a paraffin wax, a microcrystalline wax and the like; and thermoplastic resins such as an EVA, a polyethylene, a liquid polymer, an engineering plastic and the like. An amount of the binder to be added is usually 20 parts by mass or less, and preferably 15 parts by mass or less based on the total mass of the aluminum source powder, the titanium source powder, the magnesium source powder and the silicon source powder.

The above mentioned lubricant agent and plasticizer includes alcohols such as glycerin; higher fatty acids such as caplyric acid, lauric acid, palmitic acid, alginic acid, oleic acid, stearic acid and the like; and metal stearates such as aluminum stearate and the like. An amount of the lubricant agent and plasticizer to be added is usually 10 parts by mass or less, and preferably in a range from 1 part to 5 parts by mass based on the total mass of the aluminum source powder, the titanium source powder, the magnesium source powder and the silicon source powder.

The above mentioned dispersing agent includes inorganic acids such as nitric acid, hydrochloric acid, sulfuric acid and the like; organic acids such as oxalic acid, citric acid, acetic acid, malic acid, lactic acid and the like; alcohols such as methanol, ethanol, propanol and the like; surfactants such as an ammonium polycarboxylate, a polyoxyalkylene alkyl ether and the like. An amount of the dispersing agent to be added is usually 20 parts by mass or less, and preferably in a range from 2 parts to 8 parts by mass based on the total mass of the aluminum source powder, the titanium source powder, the magnesium source powder and the silicon source powder.

The above mentioned solvent includes alcohols such as methanol, ethanol, butanol, propanol and the like; glycols such as propylene glycol, a polypropylene glycol, ethylene glycol and the like; and water. Particularly, water is preferable and ion-exchanged water is more preferable due to the inclusion of less impurities. An amount of the solvent to be used is usually in a range from 10 parts to 100 parts by mass, and preferably in a range from 20 parts to 80 parts by mass based on the total mass of the aluminum source powder, the titanium source powder, the magnesium source powder and the silicon source powder.

The raw mixture can be obtained by mixing (or kneading) the aluminum source powder, the titanium source powder, the magnesium source powder and the silicon source powder, and the optional pore-forming agent and the above mentioned various additives.

The temperature at which the molding is fired is usually 1200° C. or higher, and preferably 1300° C. or higher, and usually 1700° C. or lower and preferably 1600° C. or lower. The temperature raising ratio up to the firing temperature is not particularly limited, but it is usually in a range from 1° C./hr to 500° C./hr. In the case wherein the silicon source powder is used, a temperature keeping step in a range from 1100° C. to 1300° C. for at least three hours before firing is advantageous since such step accelerates melting and diffusion of the silicon source powder. The firing step comprises a step of calcining (or degreasing) in which the binder, the pore-forming agent and the like are removed by burning them. The degreasing is typically performed during the temperature raising term (for example, in a range from 150° C. to 600° C.) leading to the firing temperature. In the degreasing step, the temperature raising speed is preferably suppressed as much as possible.

The firing is carried out in the ambient atmosphere or an atmosphere of which oxygen partial pressure is lower for the purpose of moderate burning. Depending on the kinds and the using ratio of the aluminum source powder, the titanium source powder, the magnesium source powder, the silicon source powder, binder, the pore-forming agent and the like, the firing may be carried out in an inert gas such as nitrogen, argon or the like or in a reducing gas such as carbon monoxide, hydrogen or the like. Alternatively, the firing may be carried out in an atmosphere of which steam partial pressure is lowered.

The firing is carried out in a usual firing furnace such as a tube-type electric furnace, a box-type electric furnace, a tunnel-type furnace, a far infrared furnace, a microwave heating furnace, a shaft furnace, a reverberatory furnace, a rotary furnace, a roller hearth type furnace or the like. The firing may be carried out batch-wise or continuously. The firing may be of a stationary type or a fluid type.

The firing is carried out for a period which is sufficient for the molding of the raw mixture to transit to the aluminum titanate based crystal. The period depends on the amount of the raw mixture, the type of the furnace, the firing temperature, the firing atmosphere and the like, and usually in a range from 10 minutes to 24 hours. In the manner as described above, the molding can be obtained which comprises aluminum titanate based crystal as a main component.

According to the present invention, the molding is preferably characterized in that its total pore volume is 0.1 mL/g or more, and its local maximum pore radius is 1 μm or more according to the pore volume measurement by the mercury penetration method.

The molding of the present invention has a pressure resisting strength of 5 daN or more, and a ratio of the pressure resisting strength of a molding before heating to that of a molding obtained by heating the molding at 1200° C. for 2 hours followed by immediately putting the molding in water at a normal temperature, and then drying the molding satisfies the inequality (1) shown below, and also a ratio of variation coefficients of the pressure resisting strengths of the respective moldings satisfies the inequality (2) shown below:

CSa/CSb≧0.4 (1)

CV_{CSa}/CV_{CSb}≦2.5 (2)

wherein CSa denotes a pressure resisting strength of a molding obtained by putting the molding in water at a normal temperature immediately after heating the molding at 1200° C. for 2 hours, followed by drying; CSb denotes a pressure resisting strength of a porous ceramic molding before heating; CV_{CSa} denotes a variation coefficient of a pressure resisting strength of a molding obtained by putting the molding in water at a normal temperature immediately after heating the molding at 1200° C. for 2 hours, followed by drying; and CV_{CSb} denotes a variation coefficient of a pressure resisting strength of a porous ceramic molding before heating.

### Catalyst for Production of Ethylene Oxide

The molding of the present invention can be suitably used as a catalyst carrier for the production of ethylene oxide. That is, a catalyst including said catalyst carrier and silver supported on said catalyst carrier (which catalyst is, hereinafter, sometimes referred to as an ethylene oxide catalyst) can efficiently exhibit a high catalyst performance, so that it is capable of efficiently producing ethylene oxide.

There is no particular limitation on a carrier material which forms the catalyst carrier, and for example porous refractory materials such as alumina, silicon carbide, titania, zirconia, magnesia and the like can be used. Preferably, the catalyst carrier may contain α-alumina as its main component. Specifically, α-alumina may account for 90% by weight or more of the total weight of the carrier material.

The catalyst carrier can contain silica. When silica is included, the content of silica is usually from 0.01% to 10% by weight, preferably from 0.1% to 5% by weight, and more preferably from 0.2% to 3% by weight based on the total weight of the catalyst carrier material.

The carrier material such as alumina may contain sodium. The content of sodium in the catalyst carrier is preferably 0.5% by weight or less in terms of its oxide (Na₂O). When the content of sodium in the catalyst carrier is more than the above range, since a basic site on a surface of the catalyst carrier increases, an ethylene oxide catalyst having a sufficient catalytic activity may not be obtained.

It is preferred that the catalyst carrier has water absorption of more than 10% in view of ease of impregnation with a catalyst component (silver, accelerator component described hereinafter, and the like). The higher the water absorption of the catalyst carrier, the better it is. The water absorption is more preferably 20% or more, and still more preferably 30% or more. When the water absorption of the catalyst carrier is too high, since the catalyst strength may decrease, the upper limit is usually 80% or less, and preferably 70% or less.

It is preferred that the catalyst carrier includes 0.05 mL/g or more of pores having a pore radius of 0.3 μm or more according to the measurement of a pore volume by the mercury penetration method. When the volume of pores having a pore radius of 0.3 μm or more is less than 0.05 mL/g, a sufficient catalytic activity may not be obtained.

It is preferred that the catalyst carrier has a specific surface area of 0.01 m²/g to 10 m²/g according to the measurement of a specific surface area by the nitrogen adsorption single point method. The specific surface area is more preferably from 0.1 m²/g to 5 m²/g. When the specific surface area of the catalyst carrier is less than 0.01 m²/g, since it may become difficult to support a sufficient amount of a catalyst component (silver, accelerator component described hereinafter, and the like) and also an efficiency of contact between active sites of an ethylene oxide catalyst and a gas during the production of ethylene oxide decreases, the catalytic activity tends to become insufficient. In contrast, when the specific surface area of the catalyst carrier is more than 10 m²/g, since remarkable successive oxidation of the produced ethylene oxide arises, the selectivity may deteriorate.

The ethylene oxide catalyst is obtained by supporting silver as a catalyst component on the catalyst carrier.

A supported amount of silver is preferably from 1% to 50% by weight based on the total weight of the catalyst. The supported amount is more preferably from 5% to 25% by weight, and still more preferably from 8% to 20% by weight. When the supported amount of silver is less than 1% by weight, a sufficient catalytic activity may not be obtained. In contrast, when the supporting amount is more than 50% by weight, since aggregation of silver arises, the catalytic activity may deteriorate. It is noted that the supported silver usually exists on the catalyst carrier in the form of metallic silver, and the supported amount is the weight in terms of metallic silver.

The method of supporting silver on the catalyst carrier is not particularly limited, and for example, it is possible to employ a method in which a catalyst carrier is brought into contact with or impregnated with a silver solution prepared by dissolving a silver salt, a silver compound or a silver complex in a proper solvent. The concentration of silver of the silver solution and the number of the contact or impregnation treatments may be appropriately selected so that a predetermined amount of silver is finally supported on the catalyst carrier.

It is preferred that the ethylene oxide catalyst further contains one or more kinds of accelerator components selected from the group consisting of rare earth metals, magnesium, rhenium and alkali metal in view of the improvement in the catalyst performances. When the ethylene oxide catalyst contains an alkali metal (for example, lithium, sodium, potassium, rubidium, cesium, and the like), an advantage capable of suppressing isomerization of ethylene oxide as the side reaction from arising in the vapor phase catalytic oxidation of ethylene is also obtained.

Rhenium and an alkali metal are preferable as the accelerator component, and preferable alkali metals include potassium, rubidium and cesium, the and most preferable alkali metal is cesium. Sulfur, thallium, molybdenum, tungsten, chromium and the like can be used in combination as an auxiliary accelerator. Particularly, when rhenium is used as the accelerator component, these auxiliary accelerators are suitably used in combination.

Since the contents of the accelerator component and the auxiliary accelerator vary depending on the kind, combination and difference in physical properties of the catalyst carrier, the contents may be appropriately selected and are not particularly limited. For example, the content of rhenium may be preferably from 10 ppm to 20000 ppm by weight, and more preferably from 30 ppm to 10000 ppm by weight in terms of metal, based on the total weight of the catalyst. In contrast, the content of the alkali metal is preferably from 10 ppm to 20000 ppm by weight, and more preferably from 15 ppm to 10000 ppm by weight in terms of metal, based on the total weight of the catalyst. When the alkali metal to be contained as the accelerator component is sodium and the catalyst carrier also contains sodium, it is desirable that the total content of sodium is adjusted within the above range.

In order to incorporate the accelerator component and the auxiliary accelerator, for example similarly to the silver incorporation, it is possible to employ a method in which a catalyst carrier is brought into contact with or impregnated with a solution prepared by dissolving a salt, a compound or a complex containing desired elements in a proper solvent (which is hereinafter sometimes referred to as a “solution containing an accelerator component and the like”). Upon such incorporation, the treatment in which the catalyst carrier is brought into contact with or impregnated with the solution containing an accelerator component and the like may be applied to the catalyst carrier before supporting silver, or may be carried out simultaneously with supporting silver, or may be applied to the catalyst carrier after supporting silver. Usually, it is preferred that the treatment is carried out simultaneously with supporting silver. It is noted that when rhenium is used as the accelerator component and also the above mentioned auxiliary accelerator is used in combination, it is preferred in view of the catalytic activity that the auxiliary accelerator is included (the catalyst carrier is brought into contact with or impregnated with the auxiliary accelerator solution) before supporting silver or simultaneously with supporting silver, so that silver is supported on at least a portion of the catalyst carrier, to which rhenium is then incorporated (the catalyst carrier is brought into contact with or impregnated with a rhenium solution).

When rhenium is used as the accelerator component, examples of the salt, the compound, the complex and the like, each containing rhenium which can be used for the preparation of a solution containing an accelerator component and the like include a rhenium salt such as a rhenium halide, an oxyrhenium halide, a rhenate, a perrhenate, an oxide of rhenium, an acid of rhenium and the like. Among these, a perrhenate is preferable and ammonium perrhenate is more preferable.

On the other hand, when an alkali metal is used as the accelerator component, examples of the salt, the compound and the complex, each containing the alkali metal which can be used for the preparation of solution containing an accelerator component and the like include nitrate, hydroxide, halide, carbonate, bicarbonate and oxalate carboxylate.

The solution containing an accelerator component and the like can be prepared as to each element which is used as the accelerator component or the auxiliary accelerator, and then the catalyst carrier is brought into contact with or impregnated with each solution containing an accelerator component and the like in series. It is preferred that a solution containing an accelerator component and the like in which a plurality of elements are allowed to exist in one solvent is used. It is more preferred that elements to be used as the accelerator component or the auxiliary accelerator are incorporated into a silver solution and the catalyst carrier is brought into contact with or impregnated with all together of silver, the accelerator component and the auxiliary accelerator.

If necessary, the ethylene oxide catalyst may be subjected to a firing treatment if necessary. The firing treatment may be appropriately carried out, for example, according to the conventional method in the stage where a carrier material has been molded into a carrier having a specific shape, or in the state where a carrier has been brought into contact with or impregnated with the silver solution or the solution containing an accelerator component and the like.

### Method for Producing Ethylene Oxide

In the method for producing ethylene oxide, ethylene is subjected to vapor phase catalytic oxidation using a molecular oxygen-containing gas in the presence of the ethylene oxide catalyst. Since the ethylene oxide catalyst minimizes pressure loss and has not only a large surface area but also a moderate strength when it is used for the vapor phase catalytic oxidation reaction while being packed into a reactor such as a fixed bed reactor or a reaction vessel, it can exhibit high catalyst performances and is capable of efficiently producing ethylene oxide.

The method for producing ethylene oxide can be carried out according to the conventional method except for use of the ethylene oxide catalyst, and the reaction conditions are not particularly limited. For example, the reaction temperature can be usually adjusted within a range from 150° C. to 350° C., and preferably from 200° C. to 300° C., the reaction pressure can be usually adjusted within a range from 0 kg/cm²G to 40 kg/cm²G, and preferably from 10 kg/cm²G to 30 kg/cm²G, and the space velocity can be usually adjusted within a range from 1000 hr⁻¹ to 30000 hr⁻¹ (STP), and preferably from 3000 hr⁻¹ to 8000 hr⁻¹ (STP). It is possible to use, as the raw gas to be brought into contact with the catalyst, for example, a gas which contains 0.5% to 50% by volume of ethylene, 1% to 20% by volume of oxygen, 0 to 20% by volume of carbonic acid gas (or carbon dioxide) and the balance of an inert gas (nitrogen, argon, steam and the like) and lower hydrocarbons (methane, ethane and the like), and also may contain 0.1 ppm to 50 ppm by volume of a halide such as ethylene dichloride, diphenyl chloride and the like as a reaction inhibitor. As a molecular oxygen-containing gas, air, oxygen and oxygen-enriched air and the like are usually used.

### Catalyst I for Production of Synthetic Gas

The catalyst for the production of a synthetic gas of the present invention includes a plurality of columnar portions disposed with at least one gap; and bridge portions which are disposed at least both ends in longitudinal directions of plurality of adjacent columnar portions, and joins the adjacent columnar portions to each other; and also includes through holes surrounded by the plurality of columnar portions, and openings formed on a peripheral surface by a gap between the adjacent columnar portions; the molding containing alumina as a main component, nickel being supported thereon.

A synthetic gas can be efficiently produced by using the catalyst for the production of the synthetic gas of the present invention so as to produce a synthetic gas.

As used herein, the synthetic gas is a mixed gas containing hydrogen and carbon monoxide and is industrially produced, for example, by a steam reforming method (SR method), an autothermal reforming method (ATR method), or a combined reforming method thereof using a hydrocarbon such as methane gas, natural gas, LPG, naphtha and the like as a raw material.

In the reforming method, when the hydrocarbon is methane, a mixed gas containing hydrogen and carbon monoxide (synthetic gas) is obtained by the reaction (steam reforming reaction) of the following formula (1):

CH4+H2O→CO+3H2 (1)

The resultant synthetic gas is utilized as a raw gas for the production of industrial hydrogen, ammonia, methanol, hydrocarbon liquid fuel (GTL), dimethylether, a middle- and high-calorie gas for and city gas, and the like.

In the present invention, the catalyst carrier is made of a porous refractory material containing alumina as a main component, and preferably, 90% by weight or more of the total weight of the catalyst carrier material is alumina. Herein, the crystal phase of the alumina to be used as the main component of the catalyst carrier is preferably at least one kind of χ type, κ type, ρ type, η type, γ type, pseudo γ type, δ type, θ type and α type.

The catalyst carrier (molding) preferably contains 0.1% to 30% by weight of calcium in terms of oxide (CaO). Still more preferably, at least a portion of calcium in this catalyst carrier forms a compound together with alumina. Accordingly, it is possible to suppress carbon from precipitating on a surface of the catalyst. Examples of the compound formed from calcium and alumina in the catalyst carrier include various calcium aluminates (for example, CaO.6Al2O3 (hibonite), CaO.2Al2O3, CaO.Al2O3, and the like).

In the catalyst carrier (molding), the alumina as the main component of the catalyst carrier material sometimes contains sodium. However, the content of sodium in the catalyst carrier is preferably 0.5% by weight or less in terms of oxide (Na2O). When the content of sodium in the catalyst carrier is more than the above range, since the number of a basic site on a surface of the catalyst carrier increases, sufficient catalytic activity may not be obtained upon using as the catalyst.

It is preferable that the catalyst carrier (molding) has a total pore volume of 0.20 mL/g or more and includes a pore volume of 0.05 mL/g or more of pores having radius of 0.01 μm or more according to the pore volume measurement by the mercury penetration method. When the total pore volume is less than 0.20 mL/g or the pore volume of the pores having a pore radius of 0.01 μm or more is less than 0.05 mL/g, sufficient catalytic activity may not be obtained.

The catalyst carrier (molding) preferably has a BET surface area of 1 m2/g or more according to the measurement of a specific surface area by the nitrogen adsorption single point method. More preferably, the BET surface area is from 2 m2/g to 300 m2/g. When the BET surface area of the catalyst carrier is less than 1 m2/g, since it may become difficult to support a sufficient amount of a catalyst component (nickel and the like) and also efficiency of contact between active sites of the catalyst and a raw material during the production of a synthetic gas decreases, catalytic activity tends to become insufficient.

The catalyst for the production of the synthetic gas according to the present invention is obtained by supporting nickel as a catalyst component on the catalyst carrier described above.

The supported amount of nickel is preferably from 0.1% to 50% by weight based on the total weight of the catalyst. The supporting amount of nickel is more preferably from 1% to 40% by weight, and still more preferably from 2% to 30% by weight. When the supporting amount of nickel is less than 0.1% by weight, sufficient catalytic activity may not be obtained. In contrast, when the supporting amount of nickel is more than 50% by weight, since aggregation of nickel arises, catalytic activity may deteriorate. The supported nickel usually exists on the catalyst carrier in the form of an oxide (nickel oxide), and the supporting amount is the weight in terms of metallic nickel.

The method of supporting nickel on the catalyst carrier is not particularly limited, and for example, it is possible to employ a method in which the catalyst carrier is brought into contact with or impregnated with a nickel solution prepared by dissolving a salt, a compound or a complex of nickel (nickel nitrate and the like) in a proper solvent. The concentration of nickel of the nickel solution and the number of the contact or impregnation treatments may be appropriately selected so that a predetermined amount of nickel is finally supported on the catalyst carrier. For example, when the catalyst carrier is brought into contact with or impregnated with a solution of nickel nitrate, nickel can be converted into a nickel oxide by subjecting to drying and firing thereafter, if necessary.

It is preferred that the catalyst for the production of a synthetic gas according to the present invention further contains a platinum group element so as to increase the catalytic activity. It is particularly preferred to contain, as the platinum group element, one or more kinds of elements selected from the group consisting of rhodium, ruthenium, iridium, palladium and platinum.

The content of the platinum group element is not particularly limited, and is preferably from 0.1% to 10% by weight based on the total weight of the catalyst.

In order to incorporate the platinum group element, for example, similarly to nickel, it is possible to employ a method in which a catalyst carrier is brought into contact with or impregnated with a platinum group element containing solution prepared by dissolving a salt, a compound or a complex containing desired elements in a proper solvent. Upon such incorporation, the treatment in which the catalyst carrier is brought into contact with or impregnated with the platinum group element containing solution may be applied to the catalyst carrier before supporting nickel, or may be carried out simultaneously with supporting nickel, or may be applied to the catalyst carrier after supporting nickel. Usually, it is preferred that the treatment is carried out simultaneously with supporting nickel.

When a plurality of platinum group elements are used, it is also possible that a platinum group element containing solution is prepared by every element and the carrier is sequentially brought into contact with or impregnated with each platinum group element containing solution. However, it is preferred to use a platinum group element containing solution in which the plurality of elements are allowed to exist in one solvent. It is more preferred that platinum group elements are incorporated into the nickel solution and the catalyst carrier is brought into contact with or impregnated with nickel and the plurality of platinum group elements.

It is desired that 60% or more of the supported platinum group element exist in the depth region within 1 mm from a surface of the catalyst carrier in the form of an oxide, a hydroxide or a metal.

If necessary, the catalyst for the production of the synthetic gas according to the present invention may be subjected to a firing treatment. The firing treatment may be appropriately carried out, for example, according to the conventional method in the stage where the carrier material has been molded into the catalyst carrier having a specific shape, or in the state where the catalyst carrier has been brought into contact with or impregnated with the nickel solution or the platinum group element-containing solution.

### Method for Producing Synthetic Gas

According to the method for producing the synthetic gas of the present invention, a synthetic gas (mixed gas containing carbon monoxide and hydrogen) is obtained by reacting a hydrocarbon with steam in the presence of the catalyst for the production of the synthetic gas according to the present invention. For example, when the hydrocarbon is methane, carbon monoxide and hydrogen are produced by the steam reforming reaction as shown in the above formula (1). A specific technique which can be employed in the method for producing the synthetic gas of the present invention is not particularly limited as long as it is a technique based on the steam reforming reaction of the above formula (1), and includes, for example, the steam reforming method, the autothermal reforming method, or the combined reforming method thereof. With any technique being employed, the catalyst for the production of the synthetic gas of the present invention minimizes the pressure loss, and has not only a large surface area but also a moderate strength when it is used for the production of the synthetic gas while being packed into a reactor or a reaction vessel, it can exhibit high catalyst performances and is capable of efficiently producing synthetic gas.

Hydrocarbon may be appropriately selected from one, or two or more kinds from the group of methane, ethane, propane, butane and naphtha according to the composition of the synthetic gas to be produced (a ratio of carbon monoxide to hydrogen) and are not particularly limited. For example, it is possible to use methane gas, natural gas (usually containing methane as a main component), LPG (usually containing propane or pentane as a main component), naphtha or the like.

The method for producing the synthetic gas of the present invention can be carried out according to the conventional method except for use of the catalyst for the production of a synthetic gas according to the present invention, and the reaction conditions are not particularly limited. For example, when the steam reforming method is applied, a heating furnace type reactor may be used as a reactor. The reaction temperature can be usually adjusted within a range from 400° C. to 1200° C., and preferably from 500° C. to 1100° C., and the reaction pressure can be usually adjusted within a range from 10 bar to 70 bar, and preferably from 15 bar to 60 bar. When the reaction is carried out with a fixed bed reaction system, the space velocity can be usually adjusted within a range from 1000 hr⁻¹ to 10000 hr⁻¹ (STP), and preferably from 2000 hr⁻¹ to 8000 hr⁻¹ (STP).

### Catalyst II for Production of Synthetic Gas

The molding of the present invention can be suitably used as a catalyst carrier for the production of a synthetic gas. That is, a catalyst including a catalyst carrier (molding) containing magnesia spinel as a main component and nickel supported on the catalyst carrier (which is hereinafter sometimes referred to as a synthetic gas catalyst) can efficiently exhibits high catalyst performances and is capable of efficiently producing a synthetic gas.

In the present invention, the catalyst carrier is made of a porous refractory material containing magnesia spinel as a main component. Specifically, it is preferable that 90% by weight or more of the total weight of the catalyst carrier material is magnesia spinel. Herein, magnesia spinel (MgAl₂O₄) to be used as the main component of the catalyst carrier may contain any one or both of magnesium oxide (MgO) and α-alumina (α-Al₂O₃).

Similarly to the above, it is preferred that the catalyst carrier has a total pore volume of 0.20 mL/g or more, and also has a pore volume of 0.05 mL/g or more of pores having a pore radius of 0.01 μm or more according to the pore volume measurement by the mercury penetration method.

It is preferred that the carrier has 1 m²/g or more of a specific surface area according to the measurement of the specific surface area by the nitrogen adsorption single point method. More preferably, the specific surface area is from 2 m²/g to 100 m²/g. When the specific surface area of the carrier is less than 1 m²/g, since it may become difficult to support a sufficient amount of a catalyst component (nickel and the like) and also efficiency of contact between the active sites of the catalyst and the raw material during the production of the synthetic gas decreases, catalytic activity tends to become insufficient.

The catalyst of the present invention is the carrier described above which supports nickel as a catalyst component. Similarly to the above, the supported amount of nickel is preferably from 0.1% to 50% by weight based on the total weight of the catalyst. The supported amount of nickel is more preferably from 1% to 40% by weight, and still more preferably from 2% to 30% by weight.

The others are the same as those of the above-described catalyst I for the production of the synthetic gas.

### Catalyst I for the Production of Hydrogen

The molding of the present invention can be suitably used as a catalyst carrier for the production of hydrogen. That is, a catalyst comprising the catalyst carrier (molding) which contains alumina as a main component and at least one of nickel and a platinum group element supported on the catalyst carrier (which catalyst is hereinafter sometimes referred to as catalyst for the production of hydrogen) can efficiently exhibit high catalyst performances and is capable of efficiently producing hydrogen which is used a fuel cell and the like.

There has hitherto been used, as hydrogen, hydrogen-rich reformed gas which is obtained by using various hydrocarbons such as methane gas, natural gas (city gas), propane gas, LPG, GTL synthetic liquid fuel, light oil, heavy oil, kerosene, naphtha and the like as raw materials, and reforming these hydrocarbons by the steam reforming method (SR method), the autothermal reforming method (ATR method), or the combined reforming method thereof in the presence of a catalyst. When methane is used as the raw material, such a hydrogen-rich reformed gas is obtained, for example, by carrying out the steam reforming reaction represented by the formula (1) shown below to obtain a mixed gas of hydrogen and carbon monoxide, and optionally subjecting the mixed gas to a CO conversion reaction represented by the formula (2) shown below:

CH₄+H₂O→CO+3H₂ (1)

CO+H₂O→CO₂+H₂ (2)

The catalyst carrier is made of a porous refractory material containing alumina as a main component, and it is preferable that the amount of alumina is 90% by weight or more of the total weight of the catalyst carrier material. Herein, the crystal phase of alumina to be used as the main component of the catalyst carrier is preferably one or more kinds of χ type, κ type, ρ type, η type, γ type, pseudo γ type, δ type, θ type and α type.

The alumina as the main component of the catalyst carrier material sometimes contains sodium, and the content of sodium in the catalyst carrier is preferably 0.5% by weight or less in terms of oxide (Na₂O). When the content of sodium in the catalyst carrier is more than the above range, since basic sites on a surface of the catalyst carrier increases, sufficient catalytic activity may not be obtained when used as the catalyst.

It is preferred that the catalyst carrier has a local maximum pore radius of 0.001 μm or more, and a cumulative pore volume of 0.10 mL/g or more according to the measurement of the pore volume by the mercury penetration method. When the local maximum pore radius is less than 0.001 μm or the cumulative pore volume is less than 0.10 mL/g, sufficient catalytic activity may not be obtained.

It is preferred that the catalyst carrier has a BET specific surface area of 1 m²/g or more according to the measurement of the BET specific surface area by the nitrogen adsorption single point method. More preferably, the BET specific surface area is from 2 m²/g to 300 m²/g. When the BET specific surface area of the catalyst carrier is less than 1 m²/g, it may become difficult to support a sufficient amount of a catalyst component (nickel or platinum group element) and also an efficiency of contact between the active sites of a catalyst and the raw material during the production of hydrogen may decrease, so that catalytic activity tends to become insufficient.

The catalyst for the production of hydrogen is obtained by supporting at least one of nickel and platinum group elements as a catalyst component on the catalyst carrier described above.

The supported amount of nickel is preferably from 2% to 60% by weight based on the total weight of the catalyst. The supporting amount of nickel is more preferably from 5% to 40% by weight, and still more preferably from 8% to 30% by weight. When the supporting amount of nickel is less than 2% by weight, sufficient catalytic activity may not be obtained. In contrast, when the supporting amount of nickel is more than 60% by weight, since aggregation of nickel arises, catalytic activity may deteriorate. The supported nickel usually exists on the catalyst carrier in the form of an oxide (nickel oxide), and the supported amount mentioned above is a weight in terms of nickel oxide.

The method of supporting nickel on the catalyst carrier is not particularly limited, and for example, it is possible to employ a method in which a catalyst carrier is brought into contact with or impregnated with a nickel solution prepared by dissolving a salt, a compound or a complex of nickel (nickel nitrate and the like) in a proper solvent. The concentration of nickel of the nickel solution and the number of the contact or impregnation treatment may be appropriately selected so that a predetermined amount of nickel is finally supported on the catalyst carrier. For example, when the catalyst carrier is brought into contact with or impregnated with a solution of nickel nitrate, nickel nitrate can be converted into nickel oxide by optionally drying and firing nickel nitrate.

The platinum group elements are preferably one or more kinds of elements selected from the group consisting of rhodium, ruthenium, palladium and platinum. More preferably, two or more kinds of the platinum group elements may be used in combination.

The content of the platinum group element is preferably from 0.05% to 20% by weight based on the total weight of the catalyst. The content of the platinum group element is more preferably from 0.05% to 15% by weight, and still more preferably from 0.1% to 2% by weight. When the supported amount of the platinum group element is less than 0.05% by weight, sufficient catalytic activity may not be obtained. In contrast, when the supporting amount of the platinum group element is more than 20% by weight, since aggregation of the platinum group element arises, catalytic activity may deteriorate. When two or more kinds of the platinum group elements are used, the total supported amount may be within the above range. It is noted that the supported platinum group element usually exists on the carrier in the form of an oxide, a hydroxide or a metal, and the supported amount is a weight in terms of metal.

The method of supporting the platinum element on the carrier is not particularly limited, and for example, it is possible to employ a method in which a catalyst carrier is brought into contact with or impregnated with a platinum group element containing solution prepared by dissolving a salt, a compound or a complex containing a desired element in a proper solvent, similarly to nickel. When two or more kinds of platinum group elements are used, it is also possible that a platinum group element containing solution is prepared by every element and the carrier is sequentially brought into contact with or impregnated with each platinum group element-containing solution. It is preferred to use a platinum group element containing solution in which a plurality of elements are allowed to exist in one solvent.

When both nickel and the platinum group element are supported as the catalyst components, it is also possible that the nickel containing solution and the platinum group element containing solution described above are prepared separately and the catalyst carrier is sequentially brought into contact with or impregnated with each solution. However, it is preferred that a solution containing both nickel and the platinum group element is prepared and the catalyst carrier is brought into contact with or impregnated with all of nickel and a platinum group element together.

If necessary, the catalyst for the production of hydrogen may be subjected to a firing treatment. The firing treatment may be appropriately carried out, for example, according to the conventional method in the stage where the catalyst carrier material has been molded into the carrier having the above specific shape, or in the state where the carrier has been brought into contact with or impregnated with the nickel solution, the platinum group element containing solution or the solution containing nickel and the platinum group element.

### Method for Producing Hydrogen

According to the method for producing hydrogen, a hydrocarbon is reformed by reacting the hydrocarbon with steam in the presence of the catalyst for the production of hydrogen so as to obtain a hydrogen-rich reformed gas as hydrogen. For example, when the hydrocarbon is methane, a hydrogen-rich reformed gas containing carbon monoxide is produced by the steam reforming reaction as shown in the above formula (1). A specific technique which can be employed in the method for producing hydrogen of the present invention is not particularly limited as long as it is a technique based on the steam reforming reaction of the above formula (1), and includes for example, the steam reforming method, the autothermal reforming method, or the combined reforming method thereof. Even if any technique is employed, the catalyst for the production of hydrogen minimizes the pressure loss, and has not only a large surface area but also a moderate strength when it is used for the production of a synthetic gas while being packed into a reactor or a reaction vessel, it can exhibit high catalyst performances and is capable of efficiently producing hydrogen for a fuel cell.

The hydrocarbon is not particularly limited, and for example, it is possible to use a methane gas, a natural gas (usually containing methane as a main component), a propane gas, LPG (usually containing propane and pentane as main components), liquid fuel synthesized by GTL, light oil, heavy oil, kerosene, naphtha and the like. As the hydrocarbon, one kind thereof may be used alone, or two or more kinds thereof may be used in combination.

The method for producing hydrogen can be carried out according to the conventional method except that the hydrocarbon is reformed by using the catalyst for the production of hydrogen. Therefore, the reaction conditions are not particularly limited when the hydrocarbon described above is reformed. For example, when the steam reforming method is applied, a heating furnace type reactor is used as the reactor. The reaction temperature can be usually adjusted within a range from 400° C. to 1200° C., and preferably from 500° C. to 1100° C., and the reaction pressure can be usually adjusted within a range from 10 bar to 70 bar, and preferably from 15 bar to 60 bar. When the reaction is carried out by a fixed bed reaction system, the space velocity can be usually adjusted within a range from 1000 hr⁻¹ to 10000 hr⁻¹ (STP), and preferably from 2000 hr⁻¹ to 8000 hr⁻¹ (STP).

In the method for producing hydrogen, it is possible to optionally carry out a treatment of decreasing carbon monoxide after reacting the hydrocarbon with steam as described above. Accordingly, it is possible to further increase the concentration of hydrogen and to suppress poisoning of an electrode for a fuel cell. The treatment of decreasing carbon monoxide includes, in addition to the CO conversion reaction of the formula (2), a treatment of adsorbing and separating carbon monoxide by a PSA (pressure swing adsorption) apparatus packed with an adsorbent.

### Catalyst II for Production of Hydrogen

The molding according to the present invention can be suitably used as a catalyst carrier for the production of hydrogen. That is, a catalyst including the catalyst carrier (molding) containing magnesia spinel as a main component, and at least one of nickel and platinum group elements supported on the catalyst carrier (which is hereinafter sometimes referred to as a catalyst for the production of hydrogen) can efficiently exhibit high catalyst performances and is capable of efficiently producing hydrogen used for a fuel cell and the like.

In the present invention, the catalyst carrier is made of a porous refractory material containing magnesia spinel (MgAl₂O₄) as a main component, and specifically, magnesia spinel may account for 90% by weight or more of the total weight of the catalyst carrier material. Such a carrier may contain any one or both magnesium oxide (MgO) and α-alumina α-Al₂O₃).

The others are the same as those of the catalyst I for the production of hydrogen described above.

### Dimethylether Reforming Catalyst

The molding according to the present invention can be suitably used as a catalyst carrier for reforming dimethylether. That is, a catalyst including the catalyst carrier (molding) containing alumina as a main component, and copper supported on the catalyst carrier (which is hereinafter sometimes referred to as a dimethylether reforming catalyst) can efficiently high catalyst performances and is capable of efficiently reforming dimethylether.

Dimethylether is used for the steam reforming reaction represented by the reaction formulas shown below together with a raw hydrocarbon, so as to produce various raw gases such as industrial hydrogen, ammonia, methanol and the like, and also to produce a hydrogen-containing gas used as hydrogen for a fuel cell.

Advantages of use of dimethylether as the raw hydrocarbon is that a desulfurization treatment is not necessary, and it is easy to handle (storage, transportation, etc.) since dimethylether is liquid at a normal temperature or it is liquefied at a normal temperature under a lower pressure as in the case of propane.

CH₃OCH₃+H₂O→2CH₃OH (1)

CH₃OH+H₂O→3H₂+CO₂ (2)

The catalyst carrier is made of a porous refractory material containing alumina as a main component, and specifically, alumina may account for 90% by weight or more of the total weight of the catalyst carrier material. Herein, the crystal phase of alumina to be used as the main component of the catalyst carrier is preferably one or more kinds of χ type, κ type, ρ type, η type, γ type, pseudo γ type, δ type, θ type and α type.

Alumina which is a main component of the carrier material sometimes contains sodium. The content of sodium in the catalyst carrier is preferably 0.5% by weight or less in terms of oxide (Na₂O). When the content of sodium in the catalyst carrier is more than the above range, since basic sites on a surface of the catalyst carrier increases, a catalyst having sufficient catalytic activity may not be obtained.

It is preferred that the catalyst carrier has a local maximum pore radius of 0.001 μm or more, and a cumulative pore volume of 0.10 mL/g or more according to the measurement of the pore volume by the mercury penetration method. When the local maximum pore radius is less than 0.001 μm or the cumulative pore volume is less than 0.10 mL/g, sufficient catalytic activity may not be obtained.

The catalyst carrier (molding) preferably has a BET surface area of 1 m²/g or more according to the measurement of the specific surface area by the nitrogen adsorption single point method. More preferably, the BET surface area is from 2 m²/g to 300 m²/g. When the BET surface area of the catalyst carrier is less than 1 m²/g, since it may become difficult to support a sufficient amount of a catalyst component (copper and the like) and also efficiency of contact between active sites of the catalyst and a raw material during the production of a hydrogen containing gas decreases, catalytic activity tends to become insufficient.

The dimethylether reforming catalyst is obtained by supporting copper as a catalyst component on the catalyst carrier described above. The supporting amount of copper is preferably from 1% to 50% by weight based on the total weight of the catalyst. The supported amount of copper is more preferably from 2% to 25% by weight. When the supporting amount of copper is less than 1% by weight, sufficient catalytic activity may not be obtained. In contrast, when the supported amount of copper is more than 50% by weight, catalytic activity may decrease. The support copper usually exists on the catalyst carrier in the form of metallic copper, and the supported amount is the weight in terms of metallic copper.

The method of supporting copper on the catalyst carrier is not particularly limited, and for example, it is possible to employ a method in which the catalyst carrier is brought into contact with or impregnated with a copper solution prepared by dissolving a copper salt or a copper compound in a proper solvent. The concentration of copper of the copper solution and the number of the contact or impregnation treatment may be appropriately selected so that a predetermined amount of copper is finally supported on the catalyst carrier.

It is possible to use, as the copper compound, a water-soluble salt of an organic acid such as copper acetate; and a water-soluble salt of an inorganic acid, such as copper chloride, copper sulfate, copper nitrate and the like.

It is preferable that the dimethylether reforming catalyst further contains at least any one kind of zinc, aluminum, chromium and boron so as to increase catalytic activity.

The content of zinc, aluminum, chromium and boron is not particularly limited, and it is preferably from 1% to 50% by weight based on the total weight of the catalyst.

In order to incorporate at least any one kind of zinc, aluminum, chromium and boron, for example, similarly to copper, it is possible to employ a method in which the catalyst carrier is brought into contact with or impregnated with a solution containing at least any one kind of zinc, aluminum, chromium and boron prepared by dissolving a salt, a compound or a complex containing a desired element in a proper solvent. In such method, the treatment in which the catalyst carrier is brought into contact with or impregnated with each element-containing solution described above may be applied to the catalyst carrier before supporting copper, or may be carried out simultaneously with supporting copper, or may be applied to the catalyst carrier after supporting copper. Usually, it is preferred that the treatment is carried out simultaneously with supporting copper.

When a plurality of elements are used, it is also possible that an element-containing solution is prepared by every element and the catalyst carrier is sequentially brought into contact with or impregnated with each element-containing solution. However, it is preferred to use an element-containing solution in which such a plurality of elements are allowed to exist in one solvent. It is more preferred that at least any one kind of zinc, aluminum, chromium and boron is incorporated in the above mentioned copper solution and the catalyst carrier is brought into contact with or impregnated with all of copper as well as zinc, aluminum, chromium and boron.

If necessary, the dimethylether reforming catalyst may be subjected to a firing treatment. The firing treatment may be appropriately carried out, for example, according to the conventional method in the stage where the catalyst carrier material has been molded into the catalyst carrier having the above specific shape, or in the state where the catalyst carrier has been brought into contact with or impregnated with the copper solution or at least any one kind of zinc, aluminum, chromium and boron.

### Method for Producing Hydrogen Containing Gas

According to the method for producing a hydrogen containing gas, a hydrogen containing gas (mixed gas containing carbon dioxide and hydrogen) is obtained by reacting dimethylether with steam in the presence of the dimethylether reforming catalyst described above, and carbon dioxide and hydrogen are produced by the steam reforming reaction as shown in the above formulas (1) and (2). The specific technique which can be employed in the method for producing the hydrogen containing gas is not particularly limited as long as it is a technique based on the steam reforming reaction of the above formulas (1) and (2), and may be appropriately carried out according to the conventional method. The dimethylether reforming catalyst minimizes the pressure loss and has not only a large surface area but also a moderate strength when it is used for the production of the hydrogen containing gas in the state of being packed into a reactor or a reaction vessel, it can exhibit high catalyst performances and is capable of efficiently producing a hydrogen containing gas.

The method for producing the hydrogen containing gas can be carried out according to the conventional method except for use of the catalyst according to the present invention, and the reaction conditions are not particularly limited. For example, when the steam reforming method is applied, a heating furnace type reactor is used as the reactor. The reaction temperature can be usually adjusted within a range from 100° C. to 700° C., and preferably from 150° C. to 600° C., and the reaction pressure can be adjusted to a normal pressure. When the reaction is carried out by a fixed bed reaction system, the space velocity can be usually adjusted within a range from 10 hr⁻¹ to 1000000 hr⁻¹ (STP), and preferably from 100 hr⁻¹ to 10000 hr⁻¹ (STP).

It is noted that a ratio (H₂O/DME) of steam and dimethylether (DME) to be fed to a reaction tube is from 1 to 20, and preferably from 3 to 10 in terms of a molar ratio.

### Catalyst for Production of Dimethylether

The catalyst for the production of dimethylether of the present invention is a molding including a plurality of columnar portions disposed with at least one gap; and bridge portions which are disposed at least both ends in longitudinal directions of the plurality of adjacent columnar portions, and joins the adjacent columnar portions to each other; and also including through hole surrounded by the plurality of columnar portions, and opening formed on a peripheral surface by a gap between the adjacent columnar portions; the molding containing alumina as a main component and also containing silica and a magnesium element.

It is possible to efficiently produce dimethylether by using the catalyst for the production of dimethylether according to the present invention for the production of dimethylether.

As shown in the formula shown below, dimethylether (CH₃OCH₃) is produced by the dehydration reaction of methanol (CH₃OH) in the presence of the catalyst for the production of dimethylether.

2CH₃OH→CH₃OCH₃+H₂O (I)

The catalyst for the production of dimethylether of the present invention contains alumina as a main component. Alumina is an oxide of aluminum and is usually represented by the chemical formula (1):

Al₂O₃.nH₂O [0≦n≦0.5] (1)

and an active alumina having a crystal structures such as χ, γ, η or the like is used. The active alumina may include a crystal structure other than χ, γ and η, for example, the crystal structures such as κ, δ, ρ or the like.

The content of aluminum in the catalyst for the production of dimethylether of the present invention is usually 80% by weight or more, and preferably 90% by weight or more in terms of an oxide (Al₂O₃) based on the entire catalyst for the production of dimethylether.

The catalyst for the production of dimethylether according to the present invention contains silica. By containing silica, it is possible to suppress the BET specific surface area from decreasing when the catalyst is subjected to a high temperature and high pressure steam atmosphere during the reaction.

The content of silica in the catalyst for the production of dimethylether of the present invention is preferably 0.5 parts by weight or more, and more preferably 0.8 parts by weight or more in terms of SiO₂, based on 100 parts by weight in terms of Al₂O₃. When the content of silica is less than the above range, conversion of alumina into aluminum hydroxide proceeds under a high temperature and high pressure steam atmosphere and thus the BET specific surface area of the catalyst for the production of dimethylether tends to decrease. In contrast, there is no particular limitation of the upper limit of the content of silica. However, since no further improvement of the effect of suppressing a decrease in the BET specific surface area can be expected even if silica is excessively incorporated, the upper limit of silica is usually 10 parts by weight or less, and preferably 2 parts by weight or less in terms of SiO₂, based on 100 parts by weight of alumina in terms of Al₂O₃ from an economical point of view.

There is no particular limitation on a silica source when silica is incorporated into the catalyst for the production of dimethylether of the present invention. For example, a silica sol liquid such as an acidic silica sol, a neutral silica sol and the like, a silica powder, and a silicon alkoxide such as tetraethyl orthosilicate and the like can be used. Among these silica sources, those free from metals other than aluminum and magnesium are particularly preferred.

The catalyst for the production of dimethylether of the present invention contains magnesium element. Accordingly, it becomes possible to carry out the dehydration reaction of methanol at an excellent reaction rate over a long time. The magnesium element contained in the catalyst for the production of dimethylether of the present invention is usually in the form of magnesium oxide (MgO).

The content of the magnesium element in the catalyst for the production of dimethylether of the present invention is from 0.01 parts to 1.2 parts by weight, and more preferably from 0.1 parts to 0.6 parts by weight in terms of Mg, based on 100 parts by weight of alumina in terms of Al₂O₃. When the content of the magnesium element is less than the above range, the addition effect of the magnesium element decreases and thus it may become impossible to sufficiently maintain the reaction rate when subjected to the reaction for a long time. In contrast, when the content of the magnesium element is more than the above range, the reaction rate at the beginning (initial stage) of the reaction tends to decrease and thus it may become disadvantageous in efficiently producing dimethylether.

There is no particular limitation on a magnesium source when magnesium element is incorporated into the catalyst for the production of dimethylether of the present invention. For example, it is possible to use a powder of, in addition to various magnesium salts such as magnesium sulfate, magnesium acetate, magnesium nitrate, magnesium chloride, magnesium hydroxide and the like, and magnesium oxide and the like.

The catalyst for the production of dimethylether of the present invention may contain a metallic elements other than aluminum and magnesium, for example, titanium, cerium, zirconium, zinc and the like as long as the effects of the present invention are not impaired. The metallic element is usually contained in the form of an oxide.

The catalyst for the production of dimethylether of the present invention usually contains sodium in the amount of 0.01% by weight or less in terms of oxide (Na₂O) based on the entire catalyst and, ideally, it is preferred that the catalyst does not substantially contain sodium (0% by weight). When the content of sodium is more than 0.01% by weight, the reaction rate tends to decrease.

The catalyst for the production of dimethylether of the present invention preferably has a BET specific surface area of 100 m²/g or more, and usually 300 m²/g or less, before use.

In the catalyst for the production of dimethylether of the present invention, the cumulative volume of pores having a pore radius of 1.8 nm to 100 μm is usually 0.3 cm³/g or more and usually 3.0 cm³/g or less. The cumulative volume of pores having a pore radius of 100 nm to 100 μm preferably accounts for about 10% to 60%, and more preferably about 15% to 50% of that of pores having a pore radius of 1.8 nm to 100 μm.

The catalyst for the production of dimethylether of the present invention can be produced, for example, by a method i) of sufficiently absorbing a solution (preferably an aqueous solution) containing a silica source and a magnesium source into an alumina precursor, followed by molding and further firing the precursor, or a method ii) of mixing a silica source, a magnesium source and an alumina precursor as a powder in advance, followed by molding and further firing. In any method, there is no particular limitation on the alumina precursor. Those obtained by the conventionally known method may be used, and also commercially available aluminum hydroxide and aluminum hydroxide oxide may be used. Upon firing, there is no particular limitation as to firing conditions. The firing temperature is usually adjusted from about 400° C. to 1100° C. and the firing time is usually adjusted from 2 hours to 24 hours, and the firing operation is usually carried out in an air atmosphere.

In the method i), in order that the alumina precursor absorbs the solution, it is possible to employ a manner to impregnate the alumina precursor with the solution or to coat the alumina precursor with the solution using a spray. In the method i), when the solution containing the silica source and the magnesium source is absorbed by the alumina precursor, a solution containing both the silica source and the magnesium source may be used, or a solution containing the silica source and a solution containing the magnesium source may be separately absorbed. A mixing unit in the method ii) is not particularly limited and, for example, a unit for stirring power such as a mixer may be employed, or a unit for mixing while comminuting such as a mill may be employed.

The method i) and the method ii) can also be appropriately used in combination and, for example, after mixing one of the silica source and the magnesium source as a powder with the alumina precursor, a solution of the other of the silica source and the magnesium source may be absorbed by the resultant mixture.

The method for producing the catalyst of the present invention is not limited to the above methods and can also be produced by a method of molding the alumina precursor, firing the resultant molding followed by providing the silica source and the magnesium source.

### Method for Producing Dimethylether

According to the method for producing dimethylether of the present invention, dimethylether is obtained by the dehydration reaction of methanol in the presence of the catalyst for the production of dimethylether of the present invention, and is produced by the dehydration reaction as shown in the above formula (I). A specific technique which can be employed in the method for producing dimethylether of the present invention is not particularly limited as long as it is a technique based on the dehydration reaction of the above formula (I), and may be appropriately carried out according to the conventional method. Specifically, a methanol gas generated by vaporizing methanol may be brought into contact with the catalyst at a dehydration reaction temperature. Since the catalyst of the present invention minimizes the pressure loss and has not only a large surface area but also a moderate strength when it is used for the production of dimethylether while being packed into a reactor and a reaction vessel, it can exhibit high catalyst performances and is capable of efficiently producing dimethylether.

The methanol gas may be a pure methanol gas composed entirely of methanol, but may contain water (steam) or an alcohol other than methanol, such as ethanol or isopropanol. The content of methanol relative to the total of methanol and the water and the alcohol is usually 90% by weight or more, and preferably 95% by weight or more. The methanol gas is usually used after diluting with an inert gas such as nitrogen (N₂), argon or helium. Methanol is usually vaporized by an evaporator before the reaction.

Upon the dehydration reaction of methanol, the reaction temperature is usually 250° C. or higher, preferably 270° C. or higher, and usually 450° C. or lower, preferably 400° C. or lower. The reaction pressure varies depending on the temperature, but is usually 1×10⁵ Pa or more and usually 50×10⁵ Pa or less, preferably 30×10⁵ Pa or less.

The dehydration reaction of methanol is usually carried out using a fixed bed reactor such as a multi-tubular reactor, and the gas hourly space velocity (GHSV) of methanol is usually 500 h⁻¹ or more and 150000 h⁻¹ or less.

Dimethylether obtained by the reaction can be used as it is, but may be optionally purified by the conventional method such as distillation.

### Method for Producing Ethylbenzene Dehydrogen Catalyst

The molding of the present invention can be suitably used as a catalyst carrier for the ethylbenzene dehydrogenation reaction. That is, a catalyst having a catalyst carrier (molding) containing alumina as a main component, and iron supported on the catalyst carrier (which is hereinafter sometimes referred to as an ethylbenzene dehydrogen catalyst) can efficiently exhibit high catalyst performances and is capable of efficiently accelerating the ethylbenzene dehydrogenation reaction.

The ethylbenzene dehydrogenation reaction means, for example, a reaction which produces styrene by the dehydrogenation reaction of ethylbenzene using a catalyst or the like, as shown in the following formula:

C₆H₅C₂H₅→C₆H₅C₂H₃+H₂−113 kJ/mol (I)

The catalyst carrier is made of a porous refractory material containing alumina as a main component and, specifically, alumina may account for 90% by weight or more of the total weight of the catalyst carrier material. Herein, the crystal phase of alumina to be used as the main component of the catalyst carrier is preferably one or more kinds selected from χ type, κ type, ρ type, η type, γ type, pseudo γ type, δ type, θ type and α type.

It is considered that alumina as a catalyst carrier usually has acidic sites and therefore accelerates precipitation of a carbonaceous substance and the removal of the carbonaceous substance by the water gas reaction with steam is insufficient. Therefore, it is preferred to neutralize the acidic sites by adding a basic substance to an alumina carrier, followed by a heat treatment.

The alumina carrier may be reformed with the basic substance before or after molding. When reforming is carried out before molding, an alumina powder is mixed the basic substance and kneading the mixture, followed by molding and further the heat treatment. When reforming is carried out after molding, an alumina molding may be impregnated with the basic substance so that the basic substance is supported on the alumina molding, followed by a heat treatment. These operations may be appropriately selected according to the level of water solubility of the basic substance to be used.

Examples of the basic substance used for reforming alumina include an alkali metal compound, an alkali earth metal compound, a rare earth metal compound and the like. Lithium, sodium, potassium and cesium can be used as the alkali metal, magnesium, calcium, strontium and barium can be used as the alkali earth metal, and lanthanum, cerium and the like can be used as the rare earth metal, respectively.

The supported amount of the basic substance is from 0.5% to 20% by weight, and preferably from 1.0% to 10% by weight, when all components are expressed in terms of oxides.

The molding of the carrier containing the basic substance is then fined at a temperature within a range from 300° C. to 1000° C., and preferably from 350° C. to 800° C.

An iron compound is supported on the alumina molding as the catalyst carrier containing the basic substance, followed by a heat treatment. As the iron compound, iron chloride, iron nitrate, iron hydroxide, iron sulfate and the like are used. These compounds are supported on the above alumina molded article in the form of an aqueous solution by an impregnating method, a dipping method or a spray method, followed by drying and further firing to obtain a final catalyst. The firing temperature in the preparation of the final catalyst is preferably within a range from 500° C. to 1000° C., and more preferably from 600° C. to 900° C.

The supported amount of iron in the ethylbenzene dehydrogen catalyst is preferably from 5% to 15% by weight, and more preferably from 6% to 10% by weight in terms of an oxide (Fe₂O₃), based on the total weight of the catalyst. When the supported amount of iron is less than 5% by weight, sufficient catalytic activity may not be obtained. In contrast, when the supported amount of iron is more than 15% by weight, catalytic activity may decrease.

It is preferred that at least any one kind of oxides of Cs, Mg, Ba and La is further supported on the ethylbenzene dehydrogen catalyst so as to increase the catalytic activity. The content of the oxide of Cs, Mg, Ba, La and the like is not particularly limited, but is preferably from 1% to 6% by weight, and more preferably from 2% to 5% by weight, based on the total weight of the catalyst.

At least any one kind of oxides of Cs, Mg, Ba and La may be supported before supporting an iron compound, simultaneously with supporting the iron compound, or after supporting the iron compound.

When a plurality of elements are used, it is also possible that an element containing solution is prepared by every element and the catalyst carrier is sequentially brought into contact with or impregnated with each element-containing solution. However, it is preferred to use an element containing solution in which the plurality of elements are allowed to exist in one solvent. It is also preferred that at least any one kind of oxides of Cs, Mg, Ba and La is incorporated into the above iron compound solution and the catalyst carrier is brought into contact with or impregnated with all of the iron compound and the oxides of Cs, Mg, Ba and La together.

It is preferred that the ethylbenzene dehydrogen catalyst has a local maximum pore radius of 0.001 μm or more and a cumulative pore volume of 0.10 mL/g or more according to the measurement of the pore volume by the mercury penetration method. When the local maximum pore radius is less than 0.001 μm or the cumulative pore volume is less than 0.10 mL/g, sufficient catalytic activity may not be obtained.

The ethylbenzene dehydrogen catalyst preferably has a BET specific surface area of 0.1 m²/g or more, and more preferably from 0.5 m²/g to 300 m²/g according to the measurement of the BET specific surface area by the nitrogen adsorption single point method. When the BET specific surface area of the catalyst carrier is less than 0.1 m²/g, it may becomes difficult to support a sufficient amount of a catalyst component (iron compound) and also efficiency of contact between active sites of a catalyst and a raw material during the production of styrene decreases, catalytic activity tends to become insufficient.

### Production of Styrene

According to the method for producing styrene, styrene is obtained by the dehydrogenation reaction of ethylbenzene diluted with steam in the presence of the ethylbenzene dehydrogen catalyst described above, and is produced by the dehydrogenation reaction as shown in the above formula (I). The specific technique which can be employed in the method for producing styrene is not particularly limited as long as it is a technique based on the dehydrogenation reaction of the above formula (I), and may be appropriately carried out according to the conventional method. Since the ethylbenzene dehydrogen catalyst minimizes the pressure loss and has not only a large surface area but also a moderate strength when it is used for the production of styrene while being packed into a reactor or a reaction vessel, it can exhibit high catalyst performances and is capable of efficiently producing styrene.

The method for producing styrene can be carried out according to the conventional method except for use of the ethylbenzene dehydrogen catalyst, and the reaction conditions are not particularly limited. For example, when the dehydrogenation reaction is applied, a fixed bed flow reactor is used as the reactor. The reaction temperature can be usually adjusted within a range from 400° C. to 800° C., and preferably from 500° C. to 700° C., and the reaction pressure can be usually adjusted within a range from 0 to 1 MPa, and preferably from 0.001 MPa to 0.5 MPa. The liquid hourly space velocity (LHSV) can be usually adjusted within a range from 0.1 h⁻¹ to 2.0 h⁻¹, and preferably from 0.2 h⁻¹ to 1.5 h⁻¹.

A ratio (STM/EB) of steam (STM) and ethylbenzene (EB) to be fed to a reaction tube is preferably from 1.0 to 20.0, and more preferably from 2.0 to 18.0 in terms of a molar ratio.

In such a manner, when the ethylbenzene dehydrogen catalyst is used, styrene can be efficiently produced in a high yield.

### Method for Producing Catalyst for Selective Hydrogenation

The molding according to the present invention can be suitably used as a catalyst carrier for the selective hydrogenation reaction. That is, a catalyst including the catalyst carrier (molding) containing alumina as a main component, and palladium supported on the catalyst carrier (which is hereinafter sometimes referred to as a selective hydrogenation catalyst) can efficiently exhibit high catalyst performances and is capable of efficiently accelerating the selective hydrogenation reaction.

The catalyst carrier is made of a porous refractory material containing alumina as a main component and, specifically, alumina may account for 90% by weight or more of the total weight of the catalyst carrier material. Herein, the crystal phase of alumina to be used as the main component of the catalyst carrier is preferably one or more kinds selected from χ type, κ type, ρ type, η type, γ type, pseudo γ type, δ type, θ type and α type.

It is preferred that the catalyst carrier has a local maximum pore radius of 0.001 μm or more and a cumulative pore volume of 0.10 mL/g or more according to the measurement of the pore volume by the mercury penetration method. When the local maximum pore radius is less than 0.001 μm and the cumulative pore volume is less than 0.10 mL/g, sufficient catalytic activity may not be obtained.

It is preferred that the catalyst carrier has a BET specific surface area of 0.1 m²/g or more according to the measurement of the BET specific surface area by the nitrogen adsorption single point method. When the BET specific surface area of the carrier is less than 0.1 m²/g, since it may become difficult to support a sufficient amount of a catalyst component (palladium) and also efficiency of contact between active sites of the catalyst and the raw material decreases during the purification of olefin compounds, catalytic activity tends to become insufficient.

The selective hydrogenation catalyst is obtained by supporting palladium as a catalyst component on the catalyst carrier described above. The supported amount of palladium is preferably from 0.01% to 5% by weight in terms of metallic palladium, based on the total weight of the catalyst. When the supported amount of palladium is less than 0.01% by weight, sufficient catalytic activity may not be obtained. In contrast, the supporting amount of palladium is more than 5% by weight, catalytic activity may decrease. The supported palladium usually exists on the catalyst carrier in the form of a metal, and the supported amount is the weight in terms of the metal.

The method of supporting palladium on the catalyst carrier is not particularly limited, and for example, it is possible to employ a method in which the catalyst carrier is brought into contact with or impregnated with a palladium solution prepared by dissolving a palladium salt, a palladium compound or the like in a proper solvent, followed by a heat treatment (drying and firing) and a reduction treatment. The heat treatment is usually carried out in air, and the reduction treatment is usually carried out with hydrogen in a vapor phase while heating. The concentration of palladium of the palladium solution and the number of the contact or impregnation treatment may be appropriately selected so that a predetermined amount of palladium is finally supported.

It is possible to use, as the palladium compound, water-soluble salts of organic acids, such as palladium acetate; and water-soluble salts of inorganic acids, such as palladium chloride, palladium sodium chloride, palladium sulfate, palladium nitrate, tetrachloropalladate, dichlorodiamine palladium, amine complex salts of palladium, dinitropolyamine palladiums and the like.

### Purification of Olefin Compounds by Selective Hydrogenation

According to the method for purifying olefin compounds, alkynes (hydrocarbons of acetylene series), which are highly unsaturated hydrocarbon compounds existing in a small amount in olefin compounds obtained by steam cracking of naphtha, and diolefins are selectively hydrogenated in the presence of the selective hydrogenation catalyst described above.

The olefin compound includes ethylene, propylene and butene, the acetylene-based hydrocarbon includes acetylene, methylacetylene and ethylacetylene, and the diolefin includes propadiene and butadiene.

A specific technique which can be employed in the method for purifying the olefin compounds is not particularly limited as long as it is based on the hydrogenation reaction of the reaction formula described hereinafter, and may be appropriately carried out according to the conventional method. The selective hydrogenation catalyst minimizes the pressure loss and has not only a large surface area but also a moderate strength when it is used in the method for purifying the olefin compounds while being packed into a reactor or a reaction vessel, it can exhibit high catalyst performances and is capable of efficiently purifying by removing the acetylene-based hydrocarbons and the diolefins through the selective hydrogenation.

The method for purifying the olefin compounds can be carried out according to the conventional method except for use of the selective hydrogenation catalyst. Regarding the reaction conditions, a vapor phase reaction and a liquid phase reaction are exemplified. Regarding the vapor phase reaction, a front-end system and a tail-end system are exemplified. The classification will be shown below.

The reaction formulas of the selective hydrogenation of acetylene, propadiene and methylacetylene in a mixed olefin of ethylene and propylene are shown below (vapor phase reaction, front-end system).

(Acetylene) C₂H₂+H₂→C₂H₄

(Methylacetylene) C₃H₄+H₂→C₃H₆

(Propadiene) C₃H₄+H₂→C₃H₆

A fixed bed flow reactor is used as a reactor. It is usually preferred that the reaction temperature is from 50° C. to 150° C., the reaction pressure is from 0.5 MPa to 4 MPa, and the gas hourly space velocity (GHSV) is from 4000 h⁻¹ to 8000 h⁻¹.

The reaction for the selective hydrogenation of acetylene in ethylene to ethylene after the separation of ethylene from propylene is a vapor phase reaction of the tail-end system.

A fixed bed flow reactor is used as a reaction apparatus. It is usually preferred that the reaction temperature is from 20° C. to 150° C., the reaction pressure is from 0.1 MPa to 3 MPa, the gas hourly space velocity (GHSV) is from 2000 h⁻¹ to 3500 h⁻¹, and the molar ratio of hydrogen/acetylyene to be fed to a reaction tube is from 1.0 to 3.0.

The reaction formulas of the selective hydrogenation through the vapor phase reaction or the liquid phase reaction of propadiene and methylacetylene in propylene are as follows:

(Methylacetylene) C₃H₄+H₂→C₃H₆

(Propadiene) C₃H₄+H₂→C₃H₆

In the case of the vapor phase reaction, it is usually preferred that the reaction temperature is from 50° C. to 120° C., the reaction pressure is from 0.4 MPa to 3 MPa, the gas hourly space velocity (GHSV) is from 1000 h⁻¹ to 3000 h⁻¹, and the molar ratio (hydrogen to be fed to reaction tube)/(propadiene+methylacetylene) is 3.0 or less.

In the case of the liquid phase reaction, it is usually preferred that the reaction temperature is from 20° C. to 40° C., the reaction pressure is from 2 MPa to 7 MPa, the liquid hourly space velocity (LHSV) is from 0.1 h⁻¹ to 10 h⁻¹, and the molar ratio (hydrogen to be fed to reaction tube)/(propadiene+methylacetylene) is 3.0 or less.

The selective hydrogenation reaction formulas through the selective hydrogenation of butadiene and ethylacetylene in butane, and the liquid phase reaction of dienes in cracked gasoline are as follows.

(Butadiene) C₄H₆+H₂→C₄H₈

(Ethylacetylene) C₄H₆+H₂→C₄H₈

A fixed bed flow reactor is used as a reaction apparatus. It is usually preferred that the reaction temperature is from 40° C. to 150° C., the reaction pressure is from 1 MPa to 7 MPa, the liquid hourly space velocity (LHSV) is from 0.1 h⁻¹ to 10 h⁻¹, and the volume ratio (hydrogen to be fed to reaction tube)/(liquid raw material) is from 50 to 350.

### Method for Producing Oxidation Catalyst

The molding of the present invention can be suitably used as a catalyst carrier for the oxidation reaction. A catalyst including the catalyst carrier containing alumina as a main component, and a platinum group element supported on the catalyst carrier (which is hereinafter sometimes referred to as an oxidation catalyst) can efficiently exhibit high catalyst performances and is capable of efficiently accelerating the oxidation reaction.

The catalyst carrier is made of the porous refractory material containing alumina as a main component and, specifically, alumina may account for 90% by weight or more of the total weight of the catalyst carrier material. Herein, a crystal form of alumina to be used as the main component of the catalyst carrier can be one or more kinds of crystal forms selected from boehmite type, pseudo boehmite type, χ type, κ type, ρtype, η type, γ type, pseudo γ type, δ type, θ type and α type.

It is preferred that the catalyst carrier has a local maximum pore radius of 0.001 μm or more and the cumulative pore volume of 0.10 mL/g or more in the measurement of the pore volume by the mercury penetration method. When the local maximum pore radius is less than 0.001 μm or the cumulative pore volume is less than 0.10 mL/g, sufficient catalytic activity may not be obtained.

It is preferred that the catalyst carrier has a BET specific surface area of 0.1 m²/g or more in the measurement of the BET specific surface area by the nitrogen adsorption single point method.

When the BET specific surface area of the catalyst carrier is less than 0.1 m²/g, since it may become difficult to support a sufficient amount of a catalyst component (platinum group elements) and also efficiency of contact between active sites of a catalyst and a raw material decreases during the oxidation decomposition of an exhaust gas, catalytic activity tends to become insufficient.

The oxidation catalyst is obtained by supporting a platinum group element on the catalyst carrier described above. The platinum group element is a metal selected from ruthenium, rhodium, palladium, osmium, iridium and platinum, and a catalyst obtained by supporting palladium is particularly preferred.

The supported amount of palladium is preferably from 0.01% to 50% by weight, preferably from 0.01% to 40% by weight, and more preferably from 0.01% to 20% by weight, in terms of metallic palladium, based on the total weight of the catalyst. When the supported amount of palladium is less than 0.01% by weight, sufficient catalytic activity may not be obtained. In contrast, when the supporting amount of palladium is more than 50% by weight, catalytic activity may decrease. The supported palladium usually exists on the catalyst carrier in the form of a metal, and the supported amount is the weight in terms of metal. The supported amount of the other platinum group element may the nearly the same as that of palladium.

The method of supporting palladium on the catalyst carrier is not particularly limited and, for example, it is possible to employ a method in which the catalyst carrier is brought into contact with or impregnated with a palladium solution prepared by dissolving a palladium salt or a palladium compound in a proper solvent, followed by a heat treatment (drying and firing) and a reduction treatment. The heat treatment is usually carried out in air, and the reduction treatment is usually carried out with hydrogen in a vapor phase while heating. The concentration of palladium of the palladium solution and the number of the contact or impregnation treatment may be appropriately selected so that a predetermined amount of palladium is finally supported.

It is possible to use, as the palladium compound, water-soluble salts of organic acids, such as palladium acetate; and water-soluble salts of inorganic acids, such as palladium chloride, palladium sodium chloride, palladium sulfate, palladium nitrate, tetrachloropalladate, dichlorodiamine palladium, amine complex salts of palladium, and dinitropolyamine palladiums.

### Oxidative Decomposition Method of Various Exhaust Gases

According to the oxidative decomposition method of various exhaust gases, such exhaust gases are oxidatively decomposed in the presence of the oxidation catalyst described above under the coexistence of oxygen.

A specific technique which can be employed in the oxidative decomposition method is not particularly limited as long as it is based on the oxidative decomposition reactions of the respective reaction formulas described hereinafter, and may be appropriately carried out according to the conventional method. The oxidation catalyst minimizes the pressure loss and has not only a large surface area but also a moderate strength when it is used in the oxidative decomposition method of various exhaust gases in the state of being packed into reactors or reaction vessels, it can exhibit high catalyst performances and is capable of oxidatively decomposing various exhaust gases with satisfactory efficiency.

### Oxidative Decomposition Method of Gaseous Fluorine-Containing Compound

According to the oxidative decomposition method of a gaseous fluorine-containing compound, the gaseous fluorine-containing compound as a mixture of one, or two or more kinds selected from perfluoro compounds and Freons are oxidatively decomposed in the presence of the oxidation catalyst described above under the coexistence of oxygen.

The gaseous fluorine-containing compound includes Freons, and compounds called perfluoro compounds as a general term for nitrogen fluoride, carbon fluoride, sulfur fluoride, hydrocarbon fluoride and the like.

Freons are discharged into atmospheric air from various manufacturing facilities, particularly semiconductor manufacturing sites, regardless of the concern that Freons are causative factors towards global warming. Also, the perfluoro compound, which is often used in the etching or cleaning process in semiconductor manufacturing facilities, has a large global warming potential which is at least 1000 times larger than that of carbon dioxide, and there is a very high possibility that discharge of the perfluoro compound into atmospheric air is restricted in near future as in the case of the Freons. There is also the problem that the decomposition of the perfluoro compounds are more difficult as compared with Freons.

The reaction formulas of the oxidative decomposition of methane tetrafluoride, ethane hexafluoride or propane octafluoride in a gaseous fluorine-containing compound as a mixture of one, or two or more kinds selected from perfluoro compounds and Freons are shown below:

(Decomposition of Methane Tetrafluoride)

CF₄+2H₂O→CO₂+4HF

(Decomposition of Ethane Hexafluoride)

C₂F₆+½O₂+3H₂O→2CO₂+6HF

(Decomposition of Propane Octafluoride)

C₃F₈+O₂+4H₂O→3CO₂+8HF

The reaction apparatus is not particularly limited, and a flow type reactor (a fluidized bed, a fixed bed) or a batch reactor, preferably a fixed bed flow reactor which is not of a multi-tube type is used. It is usually preferred that the reaction temperature is from 300° C. to 1000° C., and preferably from 400° C. to 900° C., the reaction pressure is from a normal pressure to 1 MPa, and the gas hourly space velocity (GHSV) is from 50000 h⁻¹ or less, and preferably from 100 h⁻¹ to 10000 h⁻¹.

The concentration of the fluorine-containing compound contained in a reaction gas is preferably adjusted to 3% by volume or less. When the concentration of the fluorine-containing compound is more than 3% by volume, the concentration is preferably adjusted to 3% by volume or less by adding a dilution gas such as air or nitrogen. This is because an adverse influence is exerted on catalyst lifetime when the concentration of fluorine-containing compound contained in the reaction gas exceeds 3% by volume. In addition, oxygen and water are incorporated into the reaction gas. An amount of oxygen is not particularly limited as long as oxygen is used in an amount required to convert carbon of the perfluoro compound into carbon dioxide and carbon monoxide, and air is the most desirable oxygen source.

Water not only functions as a component which is required to discharge halogen produced during the decomposition reaction out of the catalyst system in the form of hydrogen fluoride, but also functions to suppress aluminum in alumina from escaping from the catalyst system in the form of aluminum fluoride. When the amount of water is the same as or more than the amount and such amount is 10 times or less the amount of halogen contained in the reactant gas, that is, for example, from 4 mol to 40 mol per mol of CF₄, from 6 mol to 60 mol per mol of C₂F₆, or from 8 mol to 80 mol per mol of C₃F₈, satisfactory results can be obtained.

### Method for Oxidative Composition of Exhaust Gas Containing Carbon Monoxide and Hydrogen

The oxidative decomposition method of an exhaust gas containing carbon monoxide and hydrogen is carried out in the presence of the oxidation catalyst described above under the coexistence of oxygen.

Various gasses have recently been used in the semiconductor manufacturing process, and combustible gases such as CO and H₂ are often discharged during such process. Since CO is a combustible gas and is also harmful to the human body because of its strong toxicity, a treatment is required before releasing the gas containing the same into atmospheric air. Since H₂ is not a harmful gas but a combustible gas similarly to CO, a treatment is required.

According to the oxidative decomposition method of an exhaust gas containing carbon monoxide and hydrogen, by bringing a gas containing CO and H₂ to be treated into contact with the oxidation catalyst under the coexistence of oxygen, CO and H₂ in the gas to be treated are oxidized by the reaction shown in the formulas below.

The reaction formulas of the oxidative decomposition of carbon monoxide and hydrogen in the exhaust gas containing carbon monoxide and hydrogen are as follows.

(Decomposition of Carbon Monoxide)

CO+½O₂→CO₂

(Decomposition of Hydrogen)

H₂+½O₂→H₂O

The reaction apparatus is not particularly limited, and a flow type reactor (fluidized bed, fixed bed) or batch reactor, preferably a fixed bed flow type reactor which is not of a multi-tube type is used. It is usually preferred that the reaction temperature is from room temperature to 300° C., the reaction pressure is from a normal pressure to 1 MPa, and the gas hourly space velocity (GHSV) is from 1 h⁻¹ to 20000 h⁻¹.

In the oxidative decomposition method of an exhaust gas containing carbon monoxide and hydrogen, the oxidation of CO and H₂ with the oxidation catalyst described above is carried out under the coexistence of oxygen. It is preferred to add oxygen to the gas to be treated in an amount which is the same as that required to oxidize CO and H₂ contained in the exhaust gas, preferably about twice such amount. The addition of oxygen can be carried out by mixing air with the exhaust gas.

Oxidative Decomposition Method of Exhaust Gas Containing Volatile Organic Compound such as n-Butyl Acetate

The oxidative decomposition method of an exhaust gas containing a volatile organic compound such as n-butyl acetate is carried out in the presence of the oxidation catalyst described above under the coexistence of oxygen.

Since the exhaust gas containing a volatile organic compound such as n-butyl acetate which is discharged in the film forming process of a compound semiconductor in the semiconductor industry may cause poisoning when persons inhale a vapor having a high concentration of such organic compound, and also the volatile organic compound is a combustible gas which forms an explosive mixed gas and is likely to be charged with static electricity leading to an ignition risk, and therefore a treatment of such gas is required for the oxidative decomposition.

According to the oxidative decomposition method of an exhaust gas containing a volatile organic compound such as n-butyl acetate, by bringing into contact with the oxidation catalyst under the coexistence of oxygen, n-butyl acetate in the gas to be treated is oxidized into carbon dioxide and water by the reaction of the formula shown below.

The reaction formula of the oxidative decomposition of n-butyl acetate in the exhaust gas containing a volatile organic compound such as n-butyl acetate is shown below:

(Decomposition of N-Butyl Acetate)

CH₃COOC₄H₉+8O₂→6CO₂+6H₂O

The reaction apparatus is not particularly limited, and a flow type reactor (fluidized bed, fixed bed) or a batch reactor, preferably a flow type reactor which is not of a multi-tube type is used. It is usually preferred that the reaction temperature is from 200° C. to 400° C., preferably 250° C. to 350° C., the reaction pressure is from a normal pressure to 1 MPa, and the gas hourly space velocity (GHSV) is from 100 h⁻¹ to 1000 h⁻¹.

The component of the exhaust gas which can be treated includes, in addition to n-butyl acetate, n-octane, ethyl lactate, tetrahydrofuran and the like in the semiconductor industry. Each of these component is liquid at a normal temperature, and can be treated according to the present invention in the other fields as long as it is an organic compound which is liquid at a normal temperature.

### Oxidative Decomposition Method of Exhaust Gas Containing Organic Metal Compound

The oxidative decomposition method of an exhaust gas containing an organic metal compound is carried out in the presence of the oxidation catalyst described above under the coexistence of oxygen.

In the reaction processes of compound semiconductors, particularly MOCVD (metal organic chemical vapor deposition) and other CVD (chemical vapor phase growth, chemical vapor deposition) processes in the semiconductor industry, regarding an exhaust gas containing an organic metal compound which is discharged from the reaction process using an organic metal compound as a reaction raw material, it is not confirmed often whether or not the liquid raw materials, the solid raw materials and the organic solvents to be used as the solvents of those raw materials have highly toxic or safety. Therefore, after using such materials, it has been necessary that the exhaust gas described above is subjected to a purification treatment before release into atmospheric air.

The oxidation catalyst performs a purification treatment by the oxidative decomposition of a harmful gas containing an organic metal compound under the coexistence of oxygen, and there is no particular limitation on the organic metal compound. The oxidation catalyst can also solve problems such as upsizing of an apparatus, a post-treatment of an absorbing liquid used, and high energy cost required to maintain the combustion state as seen in a wet method and a combustion method which were commonly used in the purification treatment method of the organic metal compounds.

Oxidative Decomposition Method of Exhaust Gas Containing Nitrogen-Containing Gas such as Ammonia or Amine

According to the oxidative decomposition method of an exhaust gas containing a nitrogen-containing gas such as ammonia or an amine, the oxidative decomposition is carried out in the presence of the oxidation catalyst described above under the coexistence of oxygen.

The reaction apparatus is not particularly limited, and a flow type reactor (fluidized bed, fixed bed) or a batch reactor, preferably a fixed bed flow type reactor which is not of a multi-tubular type is used. It is usually preferred that the reaction temperature is from 150° C. to 500° C., preferably 200° C. to 400° C., the reaction pressure is from a normal pressure to 1 MPa, and the gas hourly space velocity (GHSV) is from 100 h⁻¹ to 50000 h⁻¹, and preferably from 1000 h⁻¹ to 30000 h⁻¹.

The oxidation catalyst can be used to deodorize, in addition to a nitrogen-containing gas such as ammonia or an amine, an exhaust gas containing a volatile organic compound (VOC) such as alcohols, aldehydes, ketones, hydrocarbons and carbon monoxide, for example, an exhaust gas containing ammonia and amines which is discharged from general factories and homes under the coexistence of oxygen. When the oxidation catalyst is used to deodorize exhaust gas containing 1% by volume or less, preferably 0.1% by volume or less of a nitrogen-containing component, and 1% by volume or less, preferably 0.1% by volume or less of the other volatile organic compound components, the effects of the present invention can be sufficiently exerted.

### Oxidative Decomposition Method of Oxygen-Excessive Exhaust Gas Containing Hydrocarbon

According to the oxidative decomposition method of an oxygen-excessive exhaust gas containing a hydrocarbon, an exhaust gas such as a combustion exhaust gas, which contains a hydrocarbon and also contains oxygen in an amount more excessive than an amount required for complete oxidation of a reducing substance, is oxidatively decomposed in the presence of the oxidation catalyst described above under the coexistence of oxygen.

The oxygen-excessive exhaust gas containing the hydrocarbon is discharged, for example, from thermal power stations or various factories and such exhaust gas exerts an adverse influence on the human body and environment, and therefore a purification treatment of such gas is required.

For example, the hydrocarbon is harmful since it may cause acute neurologic symptoms or chronic symptoms such as sick house syndrome when persons inhale a vapor of the hydrocarbon. Methane which is a hydrocarbon having the simplest structure is a greenhouse effect gas involved in the global warming.

According to the oxidative decomposition method of the oxygen-excessive exhaust gas containing hydrocarbon, by bringing an exhaust gas such as a combustion exhaust gas, which contains a hydrocarbon and also contains oxygen in an amount more excessive than an amount required for complete oxidation of a reducing substance, into contact with the oxidation catalyst described above under the coexistence of oxygen, the hydrocarbon in the gas to be treated is oxidized by the reaction of the formula shown below:

(Decomposition of Hydrocarbon)

CₙHₘ+(n+¼m)O₂→nCO₂+½mH₂O

When the gas to be treated is methane, the reaction scheme is as follows:

(Decomposition of Methane)

CH₄+2O₂→CO₂+2H₂O

The reaction apparatus is not particularly limited, and a flow type reactor (fluidized bed, fixed bed) or batch reactor, preferably a fixed bed flow type reactor which is not of a multi-tubular type is used. It is usually preferred that the reaction temperature is from 200° C. to 350° C., the reaction pressure is from a normal pressure to 1 MPa, and the gas hourly space velocity (GHSV) is from 1000 h⁻¹ to 10000 h⁻¹.

When the concentration of oxygen in the gas to be treated is remarkably low in the oxidative decomposition method of the oxygen-excessive exhaust gas containing a hydrocarbon, the reaction rate decreased, and therefore it is preferred that oxygen exists in the concentration is 2% or more by volume and an amount of such oxygen is 5 times or more as an oxidation equivalent of a reducing component such as a hydrocarbon in the gas. When the concentration of oxygen in the exhaust gas is not sufficiently high, a predetermined amount of air may be mixed in advance.

### Method for Producing Nitrogen Oxide Removing Catalyst

The molding according to the present invention can be suitably used as a catalyst carrier for the nitrogen oxide removing reaction. That is, a catalyst including the catalyst carrier (molding) containing alumina as a main component, and a platinum group element supported on the catalyst carrier (which is hereinafter sometimes referred to as a nitrogen oxide removing catalyst) can efficiently exhibit high catalyst performances and is capable of efficiently accelerating the oxidation reaction.

The catalyst carrier is made of a porous refractory material containing alumina as a main component and, specifically, alumina may account for 90% by weight or more of the total weight of the catalyst carrier material. Herein, a crystal form of alumina to be used as the main component of the catalyst carrier can be one or more kinds of crystal forms selected from ρ type, χ type, γ type, η type, pseudo γ type, κ type and δ type.

It is preferred that the catalyst carrier has a local maximum pore radius of 0.001 μm or more and a cumulative pore volume of 0.10 mL/g or more according to the measurement of the pore volume by the mercury penetration method. When the local maximum pore radius is less than 0.001 μm or the cumulative pore volume is less than 0.10 mL/g, sufficient catalytic activity may not be obtained.

It is preferred that the catalyst carrier has a BET specific surface area of 100 m²/g or more according to the measurement of the BET specific surface area by the nitrogen adsorption single point method.

When the BET specific surface area of the catalyst carrier is less than 100 m²/g, since it may become difficult to support a sufficient amount of a catalyst component (platinum group elements) and also efficiency of contact between active sites of the catalyst and the nitrogen oxide in the exhaust gas decreases, catalytic activity tends to become insufficient.

The nitrogen oxide removing catalyst is obtained by supporting a platinum group element on the catalyst carrier described above. The platinum group element is a metal selected from ruthenium (Ru), rhodium (Rh), palladium (Pd), osmium (Os), iridium (Ir) and platinum (Pt), and the catalyst carrier obtained by supporting palladium is particularly preferred.

The supported amount of palladium is from 0.01% to 10% by weight, preferably from 0.05% to 5% by weight, and more preferably 0.1% to 3% by weight in terms of metallic palladium, based on the total weight of the catalyst. When the supported amount of palladium is less than 0.01% by weight, sufficient catalytic activity may not be obtained. In contrast, when the supporting amount of palladium is more than 10% by weight, catalytic activity may decrease. The supported palladium usually exists on the carrier in the form of a metal, and the supported amount is a weight in terms of the metal. The supported amount of the other platinum group element may be nearly the same as that of Pd.

The method of supporting palladium on the catalyst carrier is not particularly limited, and for example, it is possible to employ a method in which the catalyst carrier is brought into contact with or impregnated with a palladium solution prepared by dissolving a palladium salt or a palladium compound in a proper solvent, followed by a heat treatment (drying and firing) and further a reduction treatment. The heat treatment is usually carried out in air, and the reduction treatment is usually carried out by hydrogen in a vapor phase while heating. The concentration of palladium of the palladium solution and the number of the contact or impregnation treatment may be appropriately selected so that a predetermined amount of palladium is finally supported.

It is possible to use, as the palladium salt or the palladium compound, water-soluble salts of organic acids, such as palladium acetate; and water-soluble salts of inorganic acids, such as palladium chloride, palladium sodium chloride, palladium sulfate, palladium nitrate, tetrachloropalladate, dichlorodiamine palladium, amine complex salts of palladium, and dinitropolyamine palladiums and the like.

The nitrogen oxide removing catalyst may contain, for example, metallic elements such as silver, iron, copper, zinc, nickel, manganese, chromium, vanadium, tungsten and molybdenum as long as the effects of the present invention are not imparted. These metallic elements are usually contained in the form of oxides.

### Method for Removing Nitrogen Oxide in Exhaust Gas

According to the method for removing nitrogen oxide in an exhaust gas, nitrogen oxide in the exhaust gas is decomposed and removed with a reducing agent in the presence of the nitrogen oxide removing catalyst described above.

Examples of the reducing agent include ammonia, hydrogen, carbon monoxide and hydrocarbons (methane series hydrocarbons) and the like described above.

That is, according to the nitrogen oxide removing method, nitrogen oxide is decomposed and removed by the reaction shown in the formulas (I) to (III) described below with the nitrogen oxide removing catalyst described above under the coexistence of the reducing agent such as ammonia. A specific technique which can be employed in the method of decomposing and removing nitrogen oxide is not particularly limited as long as it is a technique based on the selective catalytic reduction method of the formulas (I) to (VIII) described below, and may be appropriately carried out according to the conventional method. The nitrogen oxide removing catalyst minimizes the pressure loss and has not only a large surface area but also a moderate strength when it is used in the removal of nitrogen oxide in the exhaust gas while being packed into reactors or reaction vessels, it can exhibit high catalyst performances and is capable of efficiently decomposing and removing nitrogen oxide in the exhaust gas.

4NO+4NH₃+O₂→4N₂+6H₂O (I)

6NO₂+8NH₃→7N₂+12H₂O (II)

NO+NO₂+2NH₃→2N₂+3H₂O (III)

2NO+2H₂→N₂+2H₂O (IV)

2NO₂+4H₂→N₂+4H₂O (V)

2NO+2CO→N₂+2CO₂ (VI)

4NO+CH₄→2N₂+2H₂O+CO₂ (VII)

2NO₂+CH₄→N₂+2H₂O+CO₂ (VIII)

The nitrogen oxide in the exhaust gas is nitrogen monoxide, nitrogen dioxide or a mixture thereof, and the concentration thereof is usually from 0.001% to 5% by volume. It is noted that the exhaust gas contains, in addition to the nitrogen oxide, water, carbon dioxide and the like.

When nitrogen oxide in the exhaust gas is decomposed, the reaction temperature is usually 100° C. or higher, preferably 150° C. or higher, and usually 700° C. or lower, preferably 600° C. or lower. The reaction pressure is usually 1×10⁵ Pa or more and usually 50×10⁵ Pa or less, preferably 30×10⁵ Pa or less.

The decomposition reaction of the nitrogen oxide in the exhaust gas is usually carried out using a multi-tubular or non-multi-tubular fixed bed reactor. Upon using such reactor, the gas hourly space velocity (GHSV) of the exhaust gas containing nitrogen oxide is usually 100 h⁻¹ or more and 100000 h⁻¹ or less.

### Method for Producing Desulfurization Catalyst

The molding according to the present invention can be suitably used as a catalyst carrier for the hydrodesulfurization reaction. That is, a catalyst including the catalyst carrier (molding) containing alumina as a main component, and at least one, or two or more kinds of elements selected from Group VIA elements and Group VIII elements of the Periodic Table supported on the catalyst carrier (which is hereinafter sometimes referred to as a desulfurization catalyst) can efficiently exhibit high catalyst performances and is capable of efficiently accelerating the desulfurization reaction.

The catalyst carrier is made of a porous refractory material containing alumina as a main component and, specifically, alumina may account for 90% by weight or more of the total weight of the catalyst carrier material. Herein, a crystal form alumina to be used as the main component of the catalyst carrier can be one or more kinds of crystal forms selected from χ type, κ type, ρ type, η type, γ type, pseudo γ type, δ type and θ type.

It is preferred that the catalyst carrier has a BET specific surface area of 100 m²/g or more according to the measurement of the BET specific surface area by the nitrogen adsorption single point method.

When the BET specific surface area of the catalyst carrier is less than 100 m²/g, since efficiency of contact between active sites of the catalyst and a sulfur compound in a petroleum-based hydrocarbon decreases, catalytic activity tends to become insufficient.

It is preferred that the catalyst carrier has a local maximum pore radius of 0.001 μm or more and a cumulative pore volume of 0.10 mL/g or more according to the measurement of the pore volume by the mercury penetration method. When the local maximum pore radius is less than 0.001 μm or the cumulative pore volume is less than 0.10 mL/g, sufficient catalytic activity may not be obtained.

The desulfurization catalyst is obtained by supporting at least one kind of an element selected from the Group VIA elements and the Group VIII elements of the Periodic Table on the carrier described above. The element of the Group VIA of the Periodic Table is preferably a metal selected from chromium (Cr), molybdenum (Mo) and tungsten (W), and the catalyst obtained by supporting molybdenum (Mo) and/or tungsten (W) is particularly preferred. The Group VIII element of the Periodic Table is preferably a metal selected from iron (Fe), ruthenium (Ru), osmium (Os), cobalt (Co), rhodium (Rh), iridium (Ir), nickel (Ni), palladium (Pd) and platinum (Pt), and the catalyst obtained by supporting cobalt (Co) and/or nickel (Ni) is particularly preferred.

The supported amount of the Group VIA element of the Periodic Table is from 1% to 20% by weight, preferably from 2% to 18% by weight, and more preferably from 5% to 15% by weight in terms of an oxide element, based on the total weight of the catalyst. When the supported amount of the Group VIA element is 1% by weight, sufficient catalytic activity may not be obtained. In contrast, when the supporting amount of the Group VIA element is more than 20% by weight, catalytic activity may decrease. When two or more kinds of the Group VIA elements are supported, the total of each supported amount may be within the above range and, for example, a supported ratio of molybdenum to tungsten may be 1:1.

The supported amount of the Group VIII element of the Periodic Table is from 1% to 10% by weight, preferably from 2% to 8% by weight, and more preferably 3% to 7% by weight in terms of an oxide, based on the total weight of the catalyst. When the supported amount of the Group VIII element is less than 1% by weight, sufficient catalytic activity may not be obtained. In contrast, when the supported amount of the Group VIII element is more than 10% by weight, catalytic activity may decrease. When two or more kinds of Group VIII elements are supported, the total of each supporting amount may be within the above range and, for example, a supported ratio of cobalt to nickel may be 1:1.

It is noted that any one or both of the Group VIA and Group VIII elements of the Periodic Table may be supported on the catalyst carrier.

The method of supporting the Group VIA and Group VIII elements of the Periodic Table on a catalyst carrier is not particularly limited and for example, it is possible to employ a method in which the catalyst carrier is brought into contact with or impregnated with a solution prepared by dissolving a salt or a compound of molybdenum, tungsten, cobalt and nickel in a proper solvent, followed by a heat treatment (drying and firing).

The sequence of supporting the Group VIA and Group VIII elements, the concentration of the solution of the Group VIA and Group VIII elements, and the number of the contact or impregnation treatment may be appropriately selected so that a predetermined amount of the Group VIA and Group VIII elements is finally supported.

Ammonium molybdate, molybdenum trioxide, molybdic acid and the like can be used as the salt or compound of molybdenum; ammonium paratungstate, ammonium metatugstate, tungsten trioxide, tungstic acid and the like can be used as the salt or compound of tungsten; cobalt nitrate, cobalt acetate, cobalt chloride and the like can be used as the salt or compound of cobalt; and nickel nitrate, nickel sulfate, nickel chloride, nickel acetate, nickel hydrate, nickel carbonate and the like can be used as the salt or compound of nickel.

### Method for Removing Sulfur Compound in Petroleum-Based Hydrocarbon

According to the method for removing a sulfur compound in a petroleum-based hydrocarbon, the sulfur compound in the petroleum-based hydrocarbon is decomposed and removed by using the desulfurization catalyst described above under the coexistence of hydrogen.

That is, according to the method for removing the sulfur compound in the petroleum-based hydrocarbon, the sulfur compound in the petroleum-based hydrocarbon is decomposed and removed by the reaction of the formula (I) described below using the desulfurization catalyst described above under the coexistence of hydrogen. A specific technique which can be used in the method of decomposing and removing the sulfur compound in the petroleum-based hydrocarbon is not particularly limited as long as it is a technique based on the formula (I) as described below and may be carried out according to the conventional method. The catalyst for removing the sulfur compound in the petroleum-based hydrocarbon minimizes the pressure loss and has not only a large surface area but also a moderate strength when it is used to remove the sulfur compound in the petroleum-based hydrocarbon while being packed into a reactor or a reaction vessel, it can exhibit high catalyst performances and is capable of efficiently decomposing the sulfur compound in the petroleum-based hydrocarbon.

R—SH+H₂→R—H+H₂S (I)

wherein R is a hydrocarbon group.

As the petroleum-based hydrocarbon, for example, a fraction produced in the petroleum refining process of a normal pressure distillation apparatus, a vacuum distillation apparatus, a thermal decomposition treatment, a catalytic cracking treatment, a hydrogenation treatment or the like of crude oil is exemplified.

Examples of the sulfur compound in the petroleum-based hydrocarbon include thiols such as methanethiol, ethanethiol, and the like; sulfides such as dimethyl sulfide, diethyl sulfide, and the like; disulfides such as dimethyl disulfide, diethyl disulfide, and the like; thiophenes; benzothiophenes; dibenzothiophenes, benzonaphthothiophenes, and the like.

When the sulfur compound in the petroleum-based hydrocarbon is decomposed, the reaction temperature is usually 100° C. or higher, preferably 200° C. or higher, and usually 600° C. or lower, preferably 500° C. or lower. The reaction pressure is usually 1 MPa or more, preferably 2 MPa or more, and usually 20 MPa or less, preferably 15 MPa or less.

The decomposition reaction of the sulfur compound in the petroleum-based hydrocarbon is usually carried out using a multi-tubular fixed bed reactor. At that time, the liquid hourly space velocity (LHSV) of the petroleum-based hydrocarbon containing the sulfur compound is usually 0.1 h⁻¹ or more and 20 h⁻¹ or less, and the feed rate of hydrogen/raw oil is usually 0.01 Nm³/m³ or more and 2000 N/m³ or less.

### EXAMPLES

The present invention will be described specifically by way of Examples, but the present invention is not limited to these Examples. In the description in the following Examples, “parts” are by mass and ml/min representing a flow rate of a gas is based on the STP unless specifically mentioned.

### Example 1

Ammonium molybdate [(NH₄)₆Mo₇O₂₄.4H₂O] (13241 g) was dissolved in 15000 g of warm water, and the resultant liquid is designated as liquid A. Iron(III) nitrate [Fe(NO₃)₃.9H₂O] (6060 g), cobalt nitrate [Co(NO₃)₂.6H₂O] (13096 g) and cesium nitrate (CsNO₃) (585 g) were dissolved in 6000 g of warm water, then to which bismuth nitrate [Bi(NO₃)₃.5H₂O] (2910 g) was dissolved, and the resultant liquid is designated as liquid B.

While stirring the liquid A, the liquid B was added thereto so as to obtain a slurry. Subsequently, the slurry was spray-dried to obtain a dried product. A molding material obtained by mixing 100 parts by mass of the resultant dried product with 2.5 parts by mass of antimony trioxide [Sb₂O₃], 9 parts by mass of a silica-alumina fiber (RFC400-SL, manufactured by Saint-Gobain TM K.K.), 32 parts by mass of pure water and 4 parts by mass of methyl cellulose was kneaded by a kneader to obtain a pasty molding material.

Using an extrusion molding machine shown in FIG. 4(b) equipped with dies shown in FIG. 7 (diameter of first die 21: 6.4 mm, depth of grooves 21a: R 1.3 mm, number of grooves 21a: 4, outer diameter of second die 22: 30 mm, inner diameter of second die 22: 6.4 mm, depth of grooves 22a: R 1.3 mm, number of grooves 22a: 4), the pasty molding material was supplied into a flow path 25 of the dies, and then extruded at an extrusion rate of 177 mm/min while repeating the operations of rotating the first die 21 by 180 degrees at a rotational speed of 60 rpm using a motor 23, stopping the die for 1250 msec and rotating the die again by 180 degrees at a rotational speed of 60 rpm, as shown in FIG. 5. The molding obtained immediately after molding was cut into pieces each having a length of 8 to 9 mm by a piano wire to obtain moldings 10 shown in FIG. 1.

### Example 2

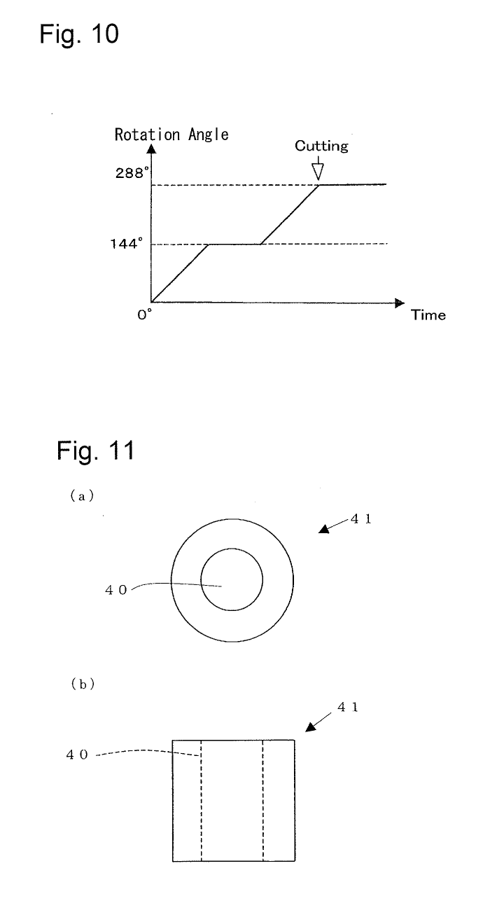

Using an extrusion molding machine shown in FIG. 4(b) equipped with dies shown in FIG. 9 (diameter of first die 21′: 6.4 mm, depth of grooves 21′ a: R 1.3 mm, number of grooves 21′ a: 5, outer diameter of second die 22′: 30 mm, inner diameter of second die 22′: 6.4 mm, the depth of grooves 22′ a: R 1.3 mm, number of grooves 22a′: 5), the pasty molding material obtained in Example 1 was supplied into a flow path 25 of the dies, and then extruded at an extrusion rate of 177 mm/min while repeating the operations of rotating the first die 21′ by 144 degrees at a rotational speed of 60 rpm using a motor 23, stopping the die for 1250 msec and rotating the die again by 144 degrees at a rotational speed of 60 rpm, as shown in FIG. 10. The molding obtained immediately after molding was cut into pieces each having a length of 8 to 9 mm by a piano wire to obtain moldings 15 shown in FIG. 8.

### Comparative Example 1

Using an extrusion molding machine shown in FIG. 4(b) equipped with dies shown in FIG. 7 (diameter of first die 21: 6.4 mm, depth of grooves 21a: R 1.3 mm, number of grooves 21a: 4, outer diameter of second die 22: 30 mm, inner diameter of second die 22: 6.4 mm, depth of grooves 22a: R 1.3 mm, number of grooves 22a: 4), the same pasty molding material as in Example 1 was supplied into a flow path 25 of the dies, and then continuously extrusion-molded at an extrusion rate of 177 mm/min while continuously rotating the first die 21 at a rotational speed of 40 rpm using a motor 23b as indicated by a dotted line in FIG. 5. Then, the resultant molding was cut into pieces each having a length of 8 to 9 mm by a piano wire in the same manner as in Example 1.

The moldings obtained in Example 1, Example 2 and Comparative Example 1 were dried in a constant-temperature constant-humidity vessel (30° C., 55% Rh) for 12 hours and then fired at 550° C. for 6 hours so as to obtain each molded article. The catalyst had a composition (excluding oxygen) of Mo₁₂Bi_{1.0}Sb_{0.5}Fe_{2.5}Co_{7.5}Cs_{0.6}.

### Example 3

### (a) Production of Catalyst for the Production of Methacrolein and Methacrylic Acid

Ammonium molybdate [(NH₄)₆Mo₇O₂₄.4H₂O] (13241 g) was dissolved in 15000 g of warm water and the resultant liquid is designated as a liquid A. Iron(III) nitrate [Fe(NO₃)₃.9H₂O] (6060 g), 13096 g of cobalt nitrate [Co(NO₃)₂.6H₂O] and 585 g of cesium nitrate (CsNO₃) were dissolved in 6000 g of warm water, then into which 2910 g of bismuth nitrate [Bi(NO₃)₃.5H₂O] was dissolved, and the resultant liquid is designated as a liquid B.

While stirring the liquid A, the liquid B was added thereto to obtain a slurry. Subsequently, the slurry was spray-dried to obtain a dried product. A molding material obtained by mixing 100 parts by mass of the resultant dried product with 2.5 parts by mass of antimony trioxide [Sb₂O₃], 9 parts by mass of a silica-alumina fiber (RFC400-SL, manufactured by Saint-Gobain TM K.K.), 32 parts by mass of pure water and 4 parts by mass of methyl cellulose was kneaded by a kneader to obtain a pasty molding material.

Using an extrusion molding machine shown in FIG. 4(b) and FIG. 7 (diameter of first die 21: 6 mm, depth of grooves 21a: R 1.5 mm, number of grooves 21a: 4, outer diameter of second die 22: 30 mm, inner diameter of second die 22: 6 mm, depth of grooves 22a: R 1.5 mm, number of grooves 22a: 4), the pasty molding material was supplied into a flow path 25 of the dies, and then extruded at an extrusion rate of 222 mm/min while repeating the operations of rotating the first die 21 by 180 degrees at a rotational speed of 90 rpm using a motor 23, stopping the die for 1200 msec and rotating the die again by 180 degrees at a rotational speed of 90 rpm. The molding obtained immediately after molding was cut into pieces each having a length of 8 to 9 mm by a piano wire to obtain catalyst precursors each having a shape shown in FIG. 1.

### (b) Firing Process

The resultant catalyst precursors were fired at 545° C. for 6 hours, and had, after firing, a composition (excluding oxygen) of Mo₁₂Bi_{0.96}Sb_{0.48}Fe_{2.4}Co_{7.2}Cs_{0.48}Si_{2.20}Al_{2.39}. to obtain catalyst raw materials.

### (c) Reduction Treatment

A glass tube was packed with the catalyst raw materials obtained in the process (b) and a mixed gas of hydrogen/nitrogen=5/95 (volume ratio) was fed at a space velocity of 240 h⁻¹, followed by a reduction treatment at 345° C. for 8 hours and further firing in air at 350° C. for 3 hours to obtain a reduction-treated catalyst.

### Comparative Example 2

The same pasty molding material as in Example 3 was molded into a shape (ring-shape) having an outer diameter of 6.4 mm, an inner diameter of 2.3 mm and a length 6 mm and including through holes 40 shown in FIG. 11 by tablet compaction or extrusion molding to obtain catalyst precursors.

Next, the resultant catalyst precursors were subjected to a firing treatment and a reduction treatment in the same manners as in Example 3 to obtain a catalyst (having a ring shape).

### Example 4

### (i) Production of Catalyst for the Production of Methacrolein and Methacrylic Acid

4414 g of ammonium molybdate [(NH₄)₆Mo₇O₂₄.4H₂O] was dissolved in 5000 g of warm water and the resultant liquid is designated to a liquid A. Separately, 2020 g of iron(III) nitrate [Fe(NO₃)₃.9H₂O], 4366 g of cobalt nitrate [Co(NO₃)₂.6H₂O] and 195 g of cesium nitrate [CsNO₃] were dissolved in 2000 g of warm water, then to which 970 g of bismuth nitrate [Bi(NO₃)₃.5H₂O] was dissolved and the resultant liquid is designated to a liquid B.

While stirring the liquid A, the liquid B was added thereto to obtain a slurry, and then this slurry was dried using a pneumatic conveying dryer to obtain a dried product. A molding material of the dried product (100 parts by mass) mixed with 6 parts by mass of a silica-alumina fiber (RFC400-SL, manufactured by Saint-Gobain TM K.K.), 33 parts by mass of pure water and 4 parts by mass of methyl cellulose was kneaded using a kneader to obtain a pasty molding material.

Using the same extrusion molding machine as in Example 3 (diameter of first die 21: 4.6 mm, depth of grooves 21a: R 1.2 mm, number of grooves 21a: 4, outer diameter of second die 22: 30 mm, inner diameter of second die 22: 4.6 mm, depth of grooves 22a: R 1.2 mm, number of grooves 22a: 4), the resultant pasty molding material was supplied into a flow path 25 of the dies, and then extruded at an extrusion rate of 222 mm/min while repeating the operations of rotating the first die 21 by 180 degrees at a rotational speed of 90 rpm using a motor 23, stopping the die for 1250 msec and rotating again by 180 degrees at a rotational speed of 90 rpm. The molding obtained immediately after molding was cut into pieces each having a length of 8 to 9 mm by a piano wire to obtain catalyst precursors having a shape shown in FIG. 1.

### (ii) Firing Process

The resultant catalyst precursors were fired at 525° C. for 6 hours. Resultant catalysts precursors contained 0.96 bismuth atoms, 2.4 iron atoms, 7.2 cobalt atoms and 0.48 cesium atoms based on 12 molybdenum atoms.

### (iii) Reduction Treatment

A glass tube was packed with the catalyst precursor material obtained in the process (ii) and a mixed gas of hydrogen/nitrogen=5/95 (volume ratio) was fed at a space velocity of 240 h⁻¹ so as to carry out a reduction treatment at 375° C. for 8 hours and further a firing treatment was carried out in air at 350° C. for 3 hours to obtain a reduction-treated catalyst.

### Example 5

A molding material obtained by mixing the dried product obtained in Example 3 (100 parts) with 32 parts of pure water, 4 parts of methyl cellulose, 9 parts of a reinforcing fiber and 2.5 parts of antimony trioxide was kneaded by a kneader to obtain a pasty molding material.

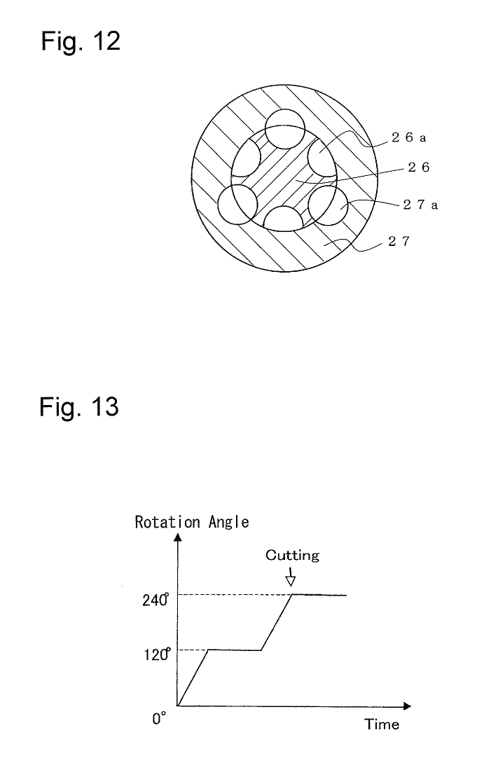

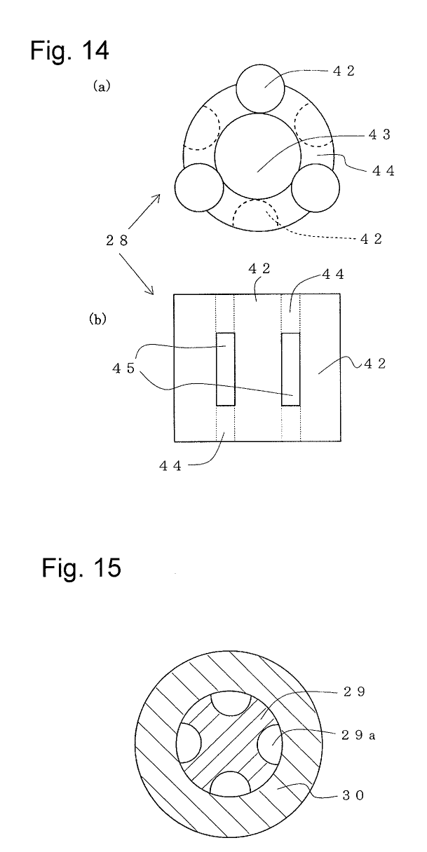

Using an extrusion molding machine shown in FIG. 4(b) equipped with dies shown in FIG. 12 (diameter of first die 26: 5.9 mm, depth of grooves 26a: R 0.8 mm, number of grooves 26a: 6, outer diameter of second die 27: 30 mm, inner diameter of second die 27: 5.9 mm, depth of grooves 27a: R 0.8 mm, number of grooves 27a: 3), the resultant pasty molding material was supplied into a flow path 25 of the dies, and then extruded at an extrusion rate of 177 mm/min while repeating the operations of rotating the first die 26 by 120 degrees at a rotational speed of 60 rpm using a motor 23, stopping the die for 1250 msec and rotating the die again by 120 degrees at a rotational speed of 60 rpm, as shown in FIG. 13. The molding obtained immediately after molding was cut into pieces each having a length of 9 to 10 mm by a piano wire to obtain moldings 28 shown in FIG. 14.

The molding 28 according to the present invention 10 shown in FIGS. 14(a) and 14(b) shows the shape which includes six columnar portions 42 disposed with a predetermined gap; and bridge portions 44 each of which is provided so as to join the adjacent columnar portions to each other at their each side ends of the two adjacent columnar portions 42 in their longitudinal directions, and; and also which includes through holes 43 surrounded by the plurality of columnar portions in the longitudinal directions of the columnar portions 42 (that is, the extrusion direction of the molding 28 as described hereinafter) and openings 45 formed on a peripheral surface (i.e. in a direction perpendicular to the extruding direction of the molding 28 as described hereinafter) by a gap between the adjacent two columnar portions 42.

In this embodiment, six columnar portions 42 are arranged at a regular interval so as to form the through holes 43 surrounded by the columnar portions. The bridge portions 44 form a circle to join the columnar portions 42 so that any two adjacent columnar portions 42 are joined to each other. Between the adjacent columnar portions 42 and 42, the opening 45 having a width corresponding to the gap therebetween is formed, and the bridge portions 41 are located above and under the opening 45 respectively.

### Example 6

A molding material obtained by mixing the dried product obtained in Example 3 (100 parts) with 32 parts of pure water, 4 parts of methyl cellulose, 9 parts of a reinforcing fiber and 2.5 parts of antimony trioxide was kneaded by a kneader to obtain a pasty molding material.

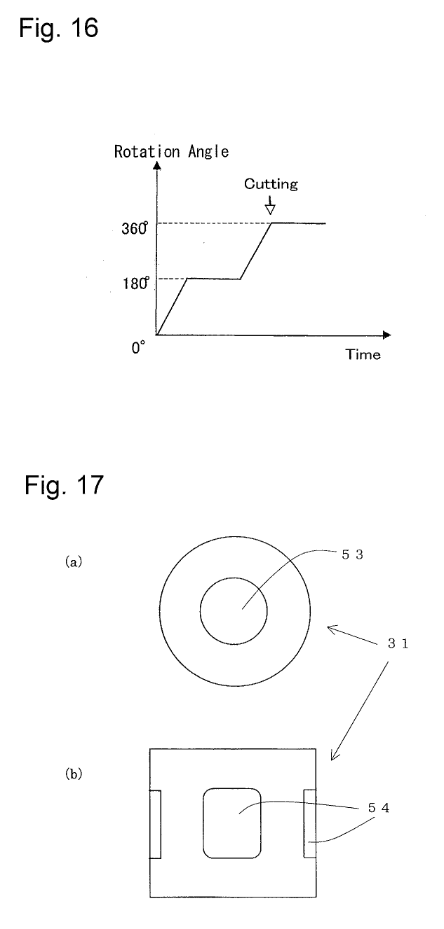

Using an extrusion molding machine shown in FIG. 4(b) equipped with dies shown in FIG. 15 (diameter of first die 29: 5.4 mm, depth of grooves 29a: R 1.3 mm, number of grooves 29a: 4, outer diameter of second die 30: 30 mm, inner diameter of second die 30: 5.4 mm), the resultant pasty molding material was supplied into a flow path 25 of the dies, and then extruded at an extrusion rate of 177 mm/min while repeating the operations of rotating the first die 29 by 180 degrees at a rotational speed of 60 rpm using a motor 23, stopping the die for 1250 msec and rotating the die again by 180 degrees at a rotational speed of 60 rpm, as shown in FIG. 16. The molding obtained immediately after molding was cut into pieces each having a length of 8 to 9 mm by a piano wire to obtain moldings 31 shown in FIG. 17.

The molding 31 shown in FIGS. 17(a) and (b) according to the present invention has a shape of a cylinder form which includes the through holes 53 in the longitudinal direction of the cylinder (i.e. the extrusion direction of the molding 31 described hereinafter), and also the openings 54 formed with a predetermined interval on a peripheral surface of the cylinder (i.e. in the direction perpendicular to the extrusion direction of the molding 28 described hereinafter).

### Example 7

A molding material obtained by mixing the dried product obtained in Example 3 (100 parts) with 33 parts of pure water, 4 parts of methyl cellulose, 18 parts of a reinforcing fiber and 2.5 parts of antimony trioxide was kneaded by a kneader to obtain a pasty molding material. The pasty molding material was extruded with the same dies as in Example 3 to obtain moldings 10 as shown in FIG. 1.

### Example 8

A molding material obtained by mixing the dried product obtained in Example 3 (100 parts) with 33 parts of pure water, 4 parts of methyl cellulose and 6 parts of a reinforcing fiber by a kneader to obtain a pasty molding material. The pasty molding material was extruded through the same dies as in Example 3 to obtain moldings 10 as shown in FIG. 1.

### Example 9

Cesium nitrate (38.2 kg), copper(II) nitrate trihydrate (10.2 kg), 85% by weight phosphoric acid (24.2 kg) and 70% by weight nitric acid (25.2 kg) were dissolved in ion-exchange water (224 kg) heated at 40° C. (which liquid is referred to as liquid A). Ammonium molybdate tetrahydrate (297 kg) was dissolved in ion-exchange water (330 kg) heated at 40° C., to which ammonium metavanadate (8.19 kg) was suspended (this liquid is referred to as liquid B). The liquid A was added dropwise in the liquid B while stirring, and then antimony trioxide (10.2 kg) was added, followed by stirring in a sealed vessel at 120° C. for 17 hours. The resultant slurry had pH of 6.3. This slurry was dried by a spray dryer. The content of ammonium nitrate in the resultant dried powder was 12% by weight. To 100 parts by weight of this dried powder, 4 parts by weight of a silica-alumina fiber (RFC400-SL, manufactured by Saint-Gobain TM K.K.), 13 parts by weight of ammonium nitrate and 9 parts by weight of ion-exchange water were added and the mixture was kneaded to obtain a pasty molding material.

Using an extrusion molding machine shown in FIG. 4(b) equipped with dies shown in FIG. 7 (diameter of first die 21: 4.6 mm, depth of grooves 21a: R 1.2 mm, number of grooves 21a: 4, outer diameter of second die 22: 30 mm, inner diameter of second die 22: 4.6 mm, depth of grooves 22a: R 1.2 mm, number of grooves 22a: 4), the resultant pasty molding material was supplied into a flow path 25 of the dies, and then extruded at an extrusion rate of 177 mm/min while repeating the operations of rotating the first die 21 by 180 degrees at a rotational speed of 60 rpm using a motor 23, stopping the die for 1250 msec and rotating the die again by 180 degrees at a rotational speed of 60 rpm, as shown in FIG. 5. The obtained molding immediately after molding was cut into pieces each having a length of 8 to 9 mm by a piano wire to obtain moldings 10 as shown in FIG. 1.

These moldings 10 were dried at a temperature of 90° C. and a humidity of 30% RH for 3 hours, and heat-treated sequentially in an air flow at 220° C. for 22 hours and then in an air flow at 250° C. for 1 hour to form a Keggin type heteropoly acid salt. This precursor was heated to 435° C. in a nitrogen gas flow, and then maintained at that temperature for 3 hours. After cooling to 300° C. in a nitrogen gas flow, and then replacing nitrogen with air, the precursor was heated in an air flow at 390° C., and then maintained at that temperature for 3 hours. After cooling to 70° C. in an air flow, a catalyst was obtained.

### Example 10

The moldings 10 obtained in Example 9 were dried at a temperature of 90° C. and a humidity of 30% RH for 3 hours, heated to 390° C. in an air flow, and then maintained at that temperature for 3 hours. After replacing air with nitrogen and heating to 435° C., the moldings were maintained at that temperature for 4 hours.

After cooling to 70° C., catalysts were obtained.

### Comparative Example 3

With the same operations as in Comparative Example 2, the molding material of Example 4 was molded into a shape (ring-shape) as shown in FIG. 11 which had an outer diameter of 6.4 mm, an inner diameter of 2.3 mm and a length 6 mm as well as a through hole 40 to obtain a catalyst precursor.

Then, the resultant catalyst precursor was subjected to a firing treatment and a reduction treatment in the same manner as in Example 4 to obtain a catalyst (ring-shape).

### Comparative Example 4

To dried powder obtained in Example 9 (100 parts by weight), 4 parts by weight of a silica-alumina fiber (RFC400-SL, manufactured by Saint-Gobain TM K.K.), 13 parts by weight of ammonium nitrate and 9 parts by weight of ion-exchange water were added, and then the mixture was kneaded to obtain a pasty molding material. Using an extrusion molding machine shown in FIG. 4(b) equipped with dies shown in FIG. 7 (diameter of first die 21: 4.6 mm, depth of grooves 21a: R 1.2 mm, number of grooves 21a: 4, outer diameter of second die 22: 30 mm, inner diameter of second die 22: 4.6 mm, depth of grooves 22a: R 1.2 mm, number of grooves 22a: 4), the pasty molding material was supplied into a flow path 25 of the dies, and then extruded at an extrusion rate of 177 mm/min while rotating the first die 21 at a rotational speed of 40 rpm using a motor 23, as indicated by a dotted line in FIG. 5. Then, the resultant molding was cut into pieces each having a length of 8 to 9 mm by a piano wire. These pieces were dried at a temperature of 90° C. and a humidity of 30% RH for 3 hours, and heat-treated sequentially in an air flow at 220° C. for 22 hours and then in an air flow at 250° C. for 1 hour to form a Keggin type heteropoly acid salt. These precursors were heated to 435° C. in a nitrogen gas flow and then maintained at the same temperature for 3 hours. After cooling to 300° C. in a nitrogen gas flow and replacing nitrogen with air, the precursors were heated in an air flow at 390° C. and then maintained at that temperature for 3 hours. After cooling to 70° C. in an air flow, catalysts were obtained.

### Example 11

A powder (26.8 parts by mass) obtained by mixing 2 parts by mass of stearic acid with 100 parts by mass of a hydraulic alumina powder at 80° C., 42.0 parts by mass of a titanium (IV) oxide powder, 15.7 parts by mass of a magnesia spinel powder, 3.4 parts by mass of a glass frit and 6.9 parts by mass of methyl cellulose were mixed. To this mixture, 34 parts of pure water, 0.35 parts of glycerin and 0.2 parts of Ceramisol (C-08, manufactured by NOF CORPORATION), and then the mixture was kneaded to obtain a pasty molding material.

Using an extrusion molding machine shown in FIG. 4(b) equipped with dies shown in FIG. 7 (diameter of first die 21: 7.8 mm, depth of grooves 21a: R 1.8 mm, number of grooves 21a: 4, outer diameter of second die 22: 11 mm, inner diameter of second die 22: 7.8 mm, depth of grooves 22a: R 1.8 mm, number of grooves 22a: 4), the resultant pasty molding material was supplied into a flow path 25 of the dies, and then extruded at an extrusion rate of 154 mm/min while repeating the operations of rotating the first die 21 by 180 degrees at a rotational speed of 90 rpm using a motor 23, stopping the die for 1000 msec and rotating the die again by 180 degrees at a rotational speed of 90 rpm, as shown in FIG. 5. The molding obtained immediately after molding was cut into pieces each having a length of 9 to 11 mm by a piano wire to obtain moldings 10 shown in FIG. 1.

The resultant moldings were dried at 120° C. for 3 hours and then dried at 1250° C. for 5 hours to obtain catalyst carriers containing a magnesium aluminum titanate-based crystal.

The molding obtained in Example 11 had a total pore volume of 0.2 mL/g and a local maximum pore radius of 1.4 μm.

### Example 12

Using an extrusion molding machine shown in FIGS. 7(a) and 7(b) (diameter of first die 21: 4.6 mm, depth of grooves 21a: R 1.2 mm, number of grooves 21a: 4, outer diameter of second die 22: 30 mm, inner diameter of second die 22: 4.6 mm, depth of grooves 22a: R 1.2 mm, number of grooves 22a: 4), the same pasty molding material as in Example 5 was supplied into a flow path 25 of the dies, and then extruded at an extrusion rate of 222 mm/min while repeating the operations of rotating the first die 21 by 180 degrees at a rotational speed of 100 rpm using a motor 23, stopping the die for 1000 msec and rotating the die again by 180 degrees at a rotational speed of 90 rpm. The molding obtained immediately after molding was cut into pieces each having a length of 8 to 9 mm by a piano wire to obtain molding precursors having a shape as shown in FIG. 1.

### Moldability

Moldability of the moldings obtained as described above was evaluated according to the following criteria:

The molding which kept its shape without causing collapse when cut immediately after molding while using a piano wire is rated “Good”, whereas the molding which was collapsed when cut immediately after molding using a piano wire is rated “Poor”. The results are shown in Table 1 below.

### Drop Strength Test

The cut moldings were dropped from an upper end of and in an iron pipe (having an inner diameter of 30.0 mm and a length of 5 m) which stood vertically and is provided with a stopper measuring 30 mm in height made of a silicone rubber at the lower end of the pipe. Subsequently, the dropped moldings were subjected to sieving so as to separate into a comminuted molding, a semi-broken molding and a non-defective molding, and then evaluation was carried out according to the proportion of the non-defective molding. Each proportion of the comminuted molding, the semi-broken molding and the non-defective molding was evaluated according to the following criteria:

Comminuted molding: 8 mesh or less (−8#) [proportion (% by mass) of moldings which passed through a sieve of 8 mesh (opening: 2.36 mm)]

Semi-broken molding: 8 mesh or more and 4 mesh or less (+8# to −4#) [proportion (% by mass) of moldings which passed through a sieve of 4 mesh (opening: 4.75 mm), and did not passed through a sieve of 8 mesh (opening: 2.36 mm)]

Non-defective molding: 4 mesh or more (+4#) [proportion (% by mass) of moldings which did not pass through a sieve of 4 mesh (opening: 4.75 mm)]

The results are shown in Table 1.

TABLE 1

| | | Drop Strength | | |
|---|---|---|---|---|
| | Moldability | +4# (%) | +8# to −4# (%) | −8# (%) |
| Example 1 | Good | 78.90 | 15.12 | 5.98 |
| Example 2 | Good | 66.71 | 22.95 | 10.34 |
| Example 3 | Good | 71.34 | 21.09 | 7.57 |
| Example 4 | Good | 75.95 | 12.04 | 12.01 |
| Example 5 | Good | 50.45 | 18.08 | 31.47 |
| Example 6 | Good | 55.82 | 28.78 | 15.39 |
| Example 7 | Good | 90.22 | 8.35 | 1.43 |
| Example 8 | Good | 97.70 | 1.45 | 0.85 |
| Example 9 | Good | 96.48 | 0.10 | 3.42 |
| Example 10 | Good | 98.91 | 0.20 | 0.89 |
| Example 11 | Good | — | — | — |
| Example 12 | Good | 85.88 | 7.38 | 6.73 |
| Comparative | Poor | 23.60 | 54.31 | 22.09 |
| Example 1 | | | | |
| Comparative | — | 96.00 | 2.54 | 1.46 |
| Example 2 | | | | |
| Comparative | — | 90.86 | 8.08 | 1.06 |
| Example 3 | | | | |
| Comparative | Poor | 40.78 | 55.24 | 3.98 |
| Example 4 | | | | |

As is apparent from the results shown in Table 1, each of non-spiral moldings obtained in Examples 1 to 10 has a higher drop strength than that of each of spiral moldings of Comparative Example 1 and Comparative Example 4.

The shapes and the sizes (or dimensions) of the catalysts obtained in Examples 1 to 10 and Comparative Examples 1 to 4 are as shown in Table 2. The bulk density shown in Table 2 was measured by the following procedure.

1. A 200 ml cylinder having an inner diameter of 36 mm is packed with 60 g of catalyst weighed accurately.

2. Tapping is carried out 100 times on a mat with a height of about 20 mm above the mat.

3. The scale of the cylinder is read out and a bulk density is calculated by the equation (2).

Bulk density=Weight(g)/Read-out Volume(ml) (2)

TABLE 2

| | | Outer | Inner | | | Bulk |
|---|---|---|---|---|---|---|
| | | diameter | diameter | Length | Cave* | Density |
| | Shape | [mm] | [mm] | [mm] | [mm] | [g/ml] |
| Example 1 | Non- | 8.39 | 3.54 | 8.97 | 3.54 | 0.94 |
| | spiral | | | | | |
| Example 2 | Non- | 7.97 | 3.48 | 7.92 | 3.48 | 0.74 |
| | spiral | | | | | |
| Example 3 | Non- | 8.09 | 2.70 | 7.80 | 2.70 | 1.00 |
| | spiral | | | | | |
| Example 4 | Non- | 6.45 | 2.03 | 8.63 | 2.03 | 0.91 |
| | spiral | | | | | |
| Example 5 | Non- | 6.31 | 4.98 | 9.11 | 4.98 | — |
| | spiral | | | | | |
| Example 6 | Non- | 5.13 | 2.66 | 10.82 | 2.66 | — |
| | spiral | | | | | |
| Example 7 | Non- | 8.19 | 2.73 | 7.87 | 2.73 | 0.85 |
| | spiral | | | | | |
| Example 8 | Non- | 8.29 | 2.76 | 7.43 | 2.76 | 0.82 |
| | spiral | | | | | |
| Example 9 | Non- | 6.25 | 1.96 | 8.26 | 1.96 | 0.79 |
| | spiral | | | | | |
| Example 10 | Non- | 6.93 | 2.18 | 8.32 | 2.18 | 0.91 |
| | spiral | | | | | |
| Example 11 | Non- | 8.52 | 7.12 | 9.14 | 7.12 | 0.82 |
| | spiral | | | | | |
| Example 12 | Non- | 6.47 | 2.03 | 8.63 | 2.03 | 0.95 |
| | spiral | | | | | |
| Comparative | Spiral | 7.91 | 3.34 | 5.66 | — | 0.76 |
| Example 1 | | | | | | |
| Comparative | Ring | 6.21 | 2.11 | 6.22 | — | 1.05 |
| Example 2 | | | | | | |
| Comparative | Ring | 5.95 | 2.15 | 5.71 | — | 0.99 |
| Example 3 | | | | | | |
| Comparative | Spiral | 6.30 | 2.01 | 6.22 | — | 0.69 |
| Example 4 | | | | | | |

*Cave: Opening which is defined by columnar portions and bridge portions connecting columnar portions on peripheral surface of molding

### Evaluation of Activity

A glass reaction tube having an inner diameter of 18 mm was packed with 3.0 ml of the catalyst obtained in Example 3, Comparative Example 2, Example 4 or Comparative Example 3 together with 30.0 g of silicon carbide (14 mesh) and a raw gas of isobutylene, oxygen, nitrogen and steam at a molar ratio of 1:2.2:6.2:2 was fed, and then the reaction was carried out under the reaction conditions of a reaction temperature of 390° C. and a space velocity SV of 1750 hr⁻¹ (Standard Temperature and Pressure (STP)). As to the catalysts obtained in Comparative Example 2, Example 4 and Comparative Example 3 were also carried out as in the case of Example 3. The results are shown in Table 3 below.

TABLE 3

| | | Selectivity to |
|---|---|---|
| | Isobutylene | Methacrolein and |
| | Conversion (%) | Methacrylic Acid (%) |
| Example 3 | 83.5 | 84.6 |
| Comparative Example 2 | 81.3 | 81.4 |
| Example 4 | 93.8 | 81.1 |
| Comparative Example 3 | 91.2 | 79.4 |

As is apparent from the results shown in Table 3, the conversion and the selectivity of the catalysts in Examples 3 and 4 were greater than those of the catalysts in Comparative Examples 2 and 3.

### Measurement of Pressure Loss

Pressure loss when a stainless steel pipe was packed with the fired moldings of Example 3 and Comparative Example 2 was measured by the following procedure. A wire net was spread over one opening of a stainless steel pipe having an inner diameter of 25 mm so as to close one opening of the pipe while the other opening was fitted with a rubber stopper which was equipped with a vent tube and a digital differential pressure manometer for pressure detection, and then the measurement was carried out. Air was passed through the tube before packing with the moldings at a flow rate of 15 L/min and a pressure difference from the atmospheric pressure was measured, and the resultant pressure difference was taken as a reference value. Subsequently, air was passed through the tube packed with the moldings in height of 1,380 mm at a flow rate of 15 L/min in the same manner as described above, and a pressure difference from the atmospheric pressure was measured using the digital differential pressure manometer. A difference in pressure (ΔP) between the resultant value and the reference value was taken as a pressure loss of the tube after packing with the moldings. The results are shown in Table 4.

TABLE 4

| | ΔP |
|---|---|
| | [mmAg.] |
| Example 3 | 179 |
| Comparative Example 2 | 308 |

(1) Pressure Resistance and Variation Coefficient Thereof of Molding Before Heating

Twenty-two moldings were picked up at random from the moldings of Example 11 and used as measurement samples. Then, a digital push-pull gauge (“Model. RX-50”, manufactured by AIKOH ENGINEERING CO., LTD.) equipped with a gauge attachment (model number: 012B) at the tip of the gauge was fixed to an electromotive stand (“Model. 1307”, manufactured by AIKOH ENGINEERING CO., LTD. After one molding was allowed to stand at the center of a lifting platform of the electromotive stand, the lifting platform with the molding was lifted at a constant speed of 60 mm/min so that the molding was pressed against the gauge attachment attached to the tip of the push-pull gauge, and then a load upon the collapse of the molding was read out with a peak holding function of the push-pull gauge. This measurement was carried out as to the twenty-two moldings, and an average of twenty measured values excluding the maximum value and the minimum value was taken as a pressure resistance (strength) CS_{b} of the moldings before heating. Similarly, a standard deviation was also calculated, and the standard deviation was divided by the pressure resistance CS_{b} of the moldings to determine a variation coefficient CV_{CSb} of the moldings before heating. It is noted that the measurement was carried out while the gauge attachment at the tip of the push-pull gauge was pressed in the direction which was perpendicular to the axial direction of the molding. The results are shown in Table 5.

(2) Pressure Resistance and Variation Coefficient Thereof of Molding after Heating

In the same manner as in the above (1), twenty-two moldings before heating were sampled from the moldings of Example 11, and put in a crucible, and then the crucible was placed in an electric furnace. After heating the crucible to 1200° C. in air at a rate of 300° C. per minute and maintaining that temperature for 2 hours. Then, a door of the electric furnace was opened, the crucible was taken out, and all of the twenty-two moldings in the crucible were immediately introduced into a stainless steel beaker containing water at a normal temperature. After separating water using a sieve having a proper opening, so as to collect moldings, which were dried by a hot air circulating type dryer at 200° C. for 3 hours. Then, the pressure resistance CSₐ and its variation coefficient CV_{CSa} were respectively determined in the same manner as in (1) above. The results are shown in Table 5.

TABLE 5

| | Variation | | Variation | | |
|---|---|---|---|---|---|
| | coefficient of | | coefficient of | | |
| Pressure resistance | pressure resistance | Pressure resistance | pressure resistance | | |
| of molding before | of molding before | of molding after | of molding after | | |
| heating, CS_{b} | heating CV_{CSb} | heating, CSₐ | heating CV_{CSa} | | |
| [daN] | [%] | [daN] | [%] | CSa/CS_{b} | CV_{csa}/CV_{csb} |
| 20.04 | 33.75 | 27.27 | 30.64 | 1.36 | 0.91 |

### CROSS-REFERENCE TO RELATED APPLICATIONS

Under the Paris Convention or any other applicable Convention, the present application claims priorities from Japanese Patent Application No. 2009-149705 filed on Jun. 24, 2009 (Title of Invention: Molding and Method for Producing the Same) as well as Japanese Patent Application No. 2009-277972 filed on Dec. 7, 2009 (Title of Invention: Molding and Method for Producing the Same, and Catalyst and Method for Producing the Same), and the contents of these applications are incorporated herein by reference thereto in their entirety.

1. A molding characterized in that it includes a plurality of columnar portions disposed with at least one gap and bridge portions each of which joins adjacent columnar portions of the plurality of columnar portions to each other at each end in the longitudinal direction of each columnar portion of the adjacent two columnar portions; and also includes through holes surrounded by the plurality of columnar portions and openings formed on a peripheral surface of the molding by gaps between the adjacent columnar portions.

2. A method for producing a molding with using an extrusion molding machine including a first die which has a plurality of grooves on its outer peripheral surface and a ring-shaped or cylindrical second die in which the first die is fitted and which has a plurality of grooves on its inner peripheral surface, characterized in that the method comprises
forming the molding by repeating:
(i) rotating at least one of the first and second dies from a position wherein at least one of the grooves of the first die is aligned with at least one of the grooves of the second die to a next position wherein at least one of the grooves of the first die is aligned with at least one of the grooves of the second die so as to form the bridge portions;
(ii) then, stopping the rotation of one of the first and second dies and forming the columnar portions; and
(iii) rotating at least one of the first and second dies again to a position wherein at least one of the grooves of the first die is aligned with at least one of the grooves of the second die to form further the bridge portions.

3. The method for producing the molding according to claim 2 characterized in that the columnar portions which have been extruded from the molding machine is cut into pieces each having a predetermined length which includes the bridge portions.

4. A catalyst for producing unsaturated aldehyde and unsaturated carboxylic acid characterized in that
it comprises a catalyst component and a molding supporting the catalyst component which molding includes a plurality of columnar portions disposed with at least one gap and bridge portions each of which joins adjacent columnar portions of the plurality of columnar portions to each other at their one ends in the longitudinal directions of the adjacent two columnar portions; and also includes through holes surrounded by the plurality of columnar portions and openings formed on a peripheral surface of the molding by gaps between the adjacent columnar portions, and
the catalyst component is a complex oxide which comprises at least molybdenum, bismuth and iron, and further comprises nickel and/or cobalt.

5. The catalyst for producing unsaturated aldehyde and unsaturated carboxylic acid according to claim 4 characterized in that
the complex oxide is one represented by the following general formula (I):
MoₐBi_{b}Fe_{c}A_{d}BₑC_{f}D_{g}Oₓ (I)
wherein Mo, Bi and Fe represents molybdenum, bismuth and iron, respectively, A represents nickel and/or cobalt, B represents an element selected from manganese, zinc, calcium, magnesium, tin and lead, C represents an element selected from phosphorus, boron, arsenic, tellurium, tungsten, antimony, silicon, aluminum, titanium, zirconium and cerium, D represents an element selected from potassium, rubidium, cesium and thallium, 0<b≦10, 0<c≦10, 1≦d≦10, 0≦e≦10, 0≦f≦10 and 0<g≦2 when a=12, and X is a value determined by the oxidation state of each element.

6. The catalyst for producing unsaturated aldehyde and unsaturated carboxylic acid according to claim 4 characterized in that the complex oxide is one obtained by firing a precursor of the complex compound in an atmosphere including molecular oxygen-containing gas and then subjecting it to a heat treatment in the presence of a reducing substance.

7. The catalyst for producing unsaturated aldehyde and unsaturated carboxylic acid according to claim 6 characterized in that the firing is carried out at a temperature in the range from 300° C. to 600° C.

8. The catalyst for producing unsaturated aldehyde and unsaturated carboxylic acid according to claim 6 characterized in that the heat treatment is carried out at a temperature in the range from 200° C. to 600° C.

9. The catalyst for producing unsaturated aldehyde and unsaturated carboxylic acid according to claim 6 characterized in that
the reducing substance is a compound selected from hydrogen, ammonia, carbon monoxide, a hydrocarbon having 1 to 6 carbon atoms, an alcohol having 1 to 6 carbon atoms, an aldehyde having 1 to 6 carbon atoms and an amine having 1 to 6 carbon atoms.

10. A method for producing unsaturated aldehyde and unsaturated carboxylic acid wherein a compound selected from propylene, isobutylene and tertiary butyl alcohol and molecular oxygen are subjected to vapor-phase catalytic oxidation in the presence of the catalyst according to claim 4.

11. A catalyst for the production of methacrylic acid characterized in that
it comprises a catalyst component and a molding supporting the catalyst component which molding includes a plurality of columnar portions disposed with at least one gap and bridge portions each of which joins adjacent columnar portions of the plurality of columnar portions to each other at their one ends in the longitudinal directions of the adjacent two columnar portions; and also includes through holes surrounded by the plurality of columnar portions and openings formed on a peripheral surface of the molding by gaps between the adjacent columnar portions, and
the catalyst component comprises a heteropoly acid compound which contains at least phosphorus and molybdenum.

12. The catalyst for the production of methacrylic acid according to claim 11 characterized in that
the heteropoly acid compound further contains vanadium, at least one element selected from potassium, rubidium, cesium and thallium, and at least element selected from copper, arsenic, antimony, boron, silver, bismuth, iron, cobalt, zinc, lanthanum and cerium.

13. The catalyst for the production of methacrylic acid according to claim 11 characterized in that
the heteropoly acid compound is obtainable by first firing of a precursor thereof under an atmosphere of non-oxidizing gas at 400° C. to 500° C. and second firing under an atmosphere of an oxidizing gas at 300° C. to 400° C.

14. The catalyst for the production of methacrylic acid according to claim 11 characterized in that
the heteropoly acid compound is obtainable by first firing of a precursor thereof under an atmosphere of an oxidizing gas at 300° C. to 400° C. and second firing under an atmosphere of a non-oxidizing gas at 400° C. to 500° C.

15. A method for producing methacrylic acid characterized in that at least one compound selected from methacrolein, isobutylaldehyde, isobutane and isobutyric acid is catalytically oxidized in a vapor phase with molecular oxygen in the presence of the catalyst according to claim 11.

16. A molding characterized in that
it includes a plurality of columnar portions disposed with at least one gap and bridge portions each of which joins adjacent columnar portions of the plurality of columnar portions to each other at their one ends in the longitudinal directions of the adjacent two columnar portions; and also includes through holes surrounded by the plurality of columnar portions and openings formed on a peripheral surface of the molding by gaps between the adjacent columnar portions, and
it comprises a aluminum titanate crystal based crystal.

17. The molding according to claim 16 characterized in that
the molding comprising the aluminum titanate based crystal is obtainable by firing a raw mixture which contains a aluminum source powder and a titanium source powder, and
a molar ratio of an amount of the aluminum source powder in terms of Al₂O₃ to that of the titanium source powder in terms of TiO₂ in the raw mixture is within a range from 35:65 to 45:55.

18. The molding according to claim 16 characterized in that
the molding comprising the aluminum titanate based crystal is obtainable by firing a raw mixture which contains a aluminum source powder, a titanium source powder and a silicon source powder,
a molar ratio of an amount of the aluminum source powder in terms of Al₂O₃ to that of the titanium source powder in terms of TiO₂ in the raw mixture is within a range from 35:65 to 45:55, and
an amount of the silicon source powder contained in the raw mixture is 5% by mass or less in inorganic components contained in the raw mixture.

19. The molding according to claim 16 characterized in that
the molding comprising the aluminum titanate based crystal is obtainable by firing a raw mixture which contains a aluminum source powder, a titanium source powder and a magnesium source powder,
a molar ratio of an amount of the aluminum source powder in terms of Al₂O₃ to that of the titanium source powder in terms of TiO₂ in the raw mixture is within a range from 35:65 to 45:55, and
a molar ratio of an amount of the magnesium source powder in terms of MgO in the raw mixture to the total of an amount of the aluminum source powder in terms of Al₂O₃ and an amount the titanium source powder in terms of TiO₂ is in a range from 0.03 to 0.15.

20. The molding according to claim 16 characterized in that
the molding comprising the aluminum titanate based crystal is obtainable by firing a raw mixture which contains a aluminum source powder, a titanium source powder, a magnesium source powder and a silicon source powder,
a molar ratio of an amount of the aluminum source powder in terms of Al₂O₃ to that of the titanium source powder in terms of TiO₂ in the raw mixture is within a range from 35:65 to 45:55, and
a molar ratio of an amount of the magnesium source powder in terms of MgO in the raw mixture to the total of an amount of the aluminum source powder in terms of Al₂O₃ and an amount the titanium source powder in terms of TiO₂ is in a range from 0.03 to 0.15, and
an amount of the silicon source powder contained in the raw mixture is 5% by mass or less based on the inorganic components contained in the raw mixture.

21. The molding according to claim 18 characterized in that the silicon source powder is a powder of feldspar or glass fit, or a mixture thereof.

22. The molding according to claim 17 characterized in that the raw mixture comprises a pore-forming agent.

23. The molding according to claim 17 characterized in that its total pore volume is 0.1 mL/g or more, and its local maximum pore radius is 1 μm or more according to the pore volume measurement by the mercury penetration method.

24. The molding according to claim 17 characterized in that a pressure resistance of the molding is 5 daN or more, and the molding satisfies the following inequality expressions (1) and (2):
CSₐ/CS_{b≧}0.4 (1)
CV_{csa}/CV_{csb}≦2.5 (2)
wherein CSₐ is a pressure resistance of the porous ceramic molding which is obtained by heating at a temperature of 1200° C. for 2 hours followed by immediately putting into water at a normal temperature and drying thereafter, CS_{b} is a pressure resistance of the molding before such heating, CV_{csa} is a variation coefficient of ratio of CSₐ, and CV_{csb} is a variation coefficient of ratio of CS_{b}.

25. A catalyst for the production of synthetic gas characterized in that
it comprises a molding which includes a plurality of columnar portions disposed with at least one gap and bridge portions each of which joins adjacent columnar portions of the plurality of columnar portions to each other at their one ends in the longitudinal directions of the adjacent two columnar portions; and also includes through holes surrounded by the plurality of columnar portions and openings formed on a peripheral surface of the molding by gaps between the adjacent columnar portions,
the molding is made of aluminum as its main component, and
nickel is supported on the molding.

26. The catalyst for the production of synthetic gas according to claim 25 characterized in that a supported amount of nickel is in a range from 0.1% to 50% by weight based on the total weight of the catalyst.

27. The catalyst for the production of synthetic gas according to claim 25 characterized in that the molding contains 0.1% to 30% by weight of calcium in terms of oxide (CaO).

28. The catalyst for the production of synthetic gas according to claim 27 characterized in that at least a portion of calcium in the molding forms a compound with aluminum.

29. The catalyst for the production of synthetic gas according to claim 25 characterized in that a crystal form of alumina is at least one of χ type, κ type, ρ type, η type, γ type, pseudo γ type, δ type, θ type and α type.

30. The catalyst for the production of synthetic gas according to claim 25 characterized in that the molding contains 0.5% by weight or less of sodium in terms of oxide (Na₂O).

31. The catalyst for the production of synthetic gas according to claim 25 characterized in that its total pore volume is 0.10 mL/g or more, and a pore volume of pores having radius 0.01 μm or more is 0.01 mL/g or more according to the pore volume measurement by the mercury penetration method.

32. The catalyst for the production of synthetic gas according to claim 25 characterized in that the molding has a BET specific surface area of 1 m²/g or more according to the measurement of the BET specific surface area by the nitrogen adsorption single point method.

33. The catalyst for the production of synthetic gas according to claim 25 characterized in that the molding further comprises a platinum group element.

34. The catalyst for the production of synthetic gas according to claim 25 characterized in that the platinum group element is at least one selected from the group consisting of rhodium, ruthenium, iridium, palladium and platinum.

35. The catalyst for the production of synthetic gas according to claim 33 characterized in that the content of the platinum group element is in a range from 0.1% to 10% by weight.

36. A process for producing synthetic gas characterized in that a hydrocarbon and steam are reacted in the presence of the catalyst for the production of synthetic gas according to claim 25.

37. A catalyst for the production of dimethylether characterized in that
it comprises a molding which includes a plurality of columnar portions disposed with at least one gap and bridge portions each of which joins adjacent columnar portions of the plurality of columnar portions to each other at their one ends in the longitudinal directions of the adjacent two columnar portions; and also includes through holes surrounded by the plurality of columnar portions and openings formed on a peripheral surface of the molding by gaps between the adjacent columnar portions,
the molding is made of aluminum as its main component, and
the molding further comprises silica and magnesium element.

38. The catalyst for the production of dimethylether according to claim 37 characterized in that the content of silica is 0.5 parts by weight or more in terms of SiO₂ to 100 parts by weight of alumina in terms of Al₂O₃.

39. The catalyst for the production of dimethylether according to claim 37 characterized in that the content of magnesium element is in a range from 0.01 parts to 1.2 parts by weight in terms of Mg to 100 parts by weight of alumina in terms of Al₂O₃.

40. A process for dimethylether characterized in that methanol is dehydrated in the presence of the catalyst for the production of dimethylether according to claim 37.
